SEQUENCE LISTING (50 sequences)

SEQ ID NO: 1 - DNA, 960 bp, Pseudomonas putida
  source 1..960: mol_type = genomic DNA
atgaagcacctgctggttggtgatattggcggcaccaatgcccgttttgcgttgtggcgtgacaaccagctgcatgaagtaaatgttttcgccaccgtggactacaccaacccggagcaggccatcgaggcctacctggaaagccaaggtatcgcccgcggtggcctggcggcggtgtgcctggcggtggccggcccggtcgatggcgatgaatttcgcttcaccaacaaccactggcgcctgagtcgcacggcattttgcaagaccttgcaggtcgagcggctgttgctgatcaatgatttcaccgcaatggcactgggcatgacgcgtctgcgcgaaggcgagttccgcgaggtgtgccccggccaggccgacccctcgaggccggcactggtgatcgggccaggtactggcctgggtgtgggctcgctgctgcgcctgggcgaacagctctggaaggccctgccgggtgaaggcgggcatgttgacctgccggtaggcaatgctcgcgaagcggcaatccaccagcagatccacagtcagatcggtcatgtcagcgccgaggccgtgctcagtgggggtggcctggtgcggctgtaccaggcgatctgtgcgctggacggcgacacgcccaggcacaagaccccggcgcatatcaccgatgccgcgctgggcggggagccacgggcgctggcggtggtcgagcagttctgtcgtttccttgggcgagtggcgggtaataatgtgctgacgctgggcgcgcgaggcggggtctatattgtgggcggtgtgattccgcgctttgccgagctgttcctgcgtagcgggtttgccgcgagttttgccgacaaggggtgtatgagtggctatttcaccggggtgccggtgtggctggtgacggcggagttttccgggttggagggagcaggggtagcgttgcagcaggcgttggatcactga

SEQ ID NO: 2 - protein, 319 aa, Pseudomonas putida
MKHLLVGDIGGTNARFALWRDNQLHEVNVFATVDYTNPEQAIEAYLESQGIARGGLAAVCLAVAGPVDGDEFRFTNNHWRLSRTAFCKTLQVERLLLINDFTAMALGMTRLREGEFREVCPGQADPSRPALVIGPGTGLGVGSLLRLGEQLWKALPGEGGHVDLPVGNAREAAIHQQIHSQIGHVSAEAVLSGGGLVRLYQAICALDGDTPRHKTPAHITDAALGGEPRALAVVEQFCRFLGRVAGNNVLTLGARGGVYIVGGVIPRFAELFLRSGFAASFADKGCMSGYFTGVPVWLVTAEFSGLEGAGVALQQALDH

SEQ ID NO: 3 - DNA, 2412 bp, Pseudomonas putida
  source 1..2412: mol_type = genomic DNA
atgagcactgaaggtgcgaaccaaggaagccgctggctaccgcgcctgattggcgcgctgctgttgctgatgggcctggccctgctggccggcggtatcaagctgagccagctgggcggatcgctgtactacctgatcgccggtattggctttgccctttcgggcgtcctgctgctggcccaacgccagatcgccctgggcctgtatggcctggtgctgctgggcagcaccgtgtgggccctgttcgaagtaggcctggactggtggcaactggtgccacggctggctatctggttcgccatcggcgtggtgttgctgctgccgtgggcacgtcgcccgctgatcggcccagccagcaaagccaacactgcactgctcggcgtggcagtggtcgcttcgggcgcttgcgcgctggccagccagttcacccatcccggtgaagtgttcggcgaactgggccgcgacagcagcgaaatggccagcgccgccccggccatgcccgacggcgaatggcaggcctacggccgtaccgagcatggcgaccgctactcgccgctgcgccagatcaccccgcagaacgcctaccgtctggaagaagcctggcgcattcgcaccggtgatctgccaaccgaaaacgacccggtggagctgaccaaccagaacaccccgctgaaggtcaacggcatgctttacgcctgcaccgcgcacagtcgcctgctggcgctggacccggacactggcgcagaaatctggcgctacgacccgcaggtcaagagcccgaccggcaccttcaagggctttgcccacatgacctgccgtggcgtctcgtactatgacgaaaaccgctacgtcagccgcgacggcagcccggcgccgaaaattaccgatgcaggccaggccgtggcccaagcctgcccgcgtcgcctctatctgcctaccgcggatgcccgcctgatcgccatcaacgccgacaacggcaaggtctgcgaaggcttcgccaaccagggcgtgatcgacctcaccaccggcattggcccattcaccgccggcggctattactccacctcgcctgctgcgattacccgtgacctggtgatcatcggtggccacgtcaccgacaacgagtcgaccaatgagccgtccggggtgatccgcgcctacgacgtgcacgacggccacctggtgtggaactgggacagtaacaacccggacgacaccaagccattggctgccggcaaaatgtacagccgcaactcggccaacatgtggtcgatcgccagcgtcgacgaagaccttggcatgatctacctgccgctgggcaaccagaccccggaccagtggggcgccgaccgcaccccgggcgccgagaagtacagcgccggcgtggtcgcccttgacctggccaccggcaaggcacgctggaactatcagttcacccaccacgacctgtgggacatggacgtcggcagccagccgaccctggtacacctgaaaaccgacgatggtgtgaaaccggcgatcatcgtaccgaccaagcaaggcagcctgtacgtgctcgaccgccgcgacggtacgccaatcgtgccgatccgcgagatccccaccccacaaggcgcagtggaaggcgaccacacctcgcccacccaggcccgctccgacctcaacctgctcggcccagagctgaccgaacaggccatgtggggcgccacgcctttcgaccagatgctgtgccgcatccagttccgcgaactgcgctacgaaggccagtacaccccgccatccgaacaaggttcgttggtctaccccggcaacgtcggtgtattcaactggggcagcgtgtcggtcgacccggtgcgccagctgctgttcacttcgcccaactacatggcgttcgtgtcgaagatggtcccgcgtgagcaggttgccgaaggcagcaagcgcgaaagcgagaccagcggcgtgcagccgaacaccggcgcaccgtatgcagtgatcatgcacccgttcatgtcgccgctcggtgtaccgtgccaggcacccgcctggggctacgtcgccgccatcgacctgttcaccaacaaggtggtgtggaaacacaagaacggcaccacccgcgacagcaccccgctaccgatcggcctgccggttggcgtgccgagcatgggtggctcgatcgtcaccgccggtggcgtcggcttcctcagcggcacgctcgaccagtacctgcgcgcctatgacgtgaacaacggcaaggagttgtggaaagcacgcctgccagcgggtggccaggctacgccgatgagctacaccggcaaggacggcaagcagtacgtgctggtgactgccggcggccatggctcgctgggcaccaagatgggcgattacatcattgcttacaaattagccgagtaa

SEQ ID NO: 4 - protein, 803 aa, Pseudomonas putida
MSTEGANQGSRWLPRLIGALLLLMGLALLAGGIKLSQLGGSLYYLIAGIGFALSGVLLLAQRQIALGLYGLVLLGSTVWALFEVGLDWWQLVPRLAIWFAIGVVLLLPWARRPLIGPASKANTALLGVAVVASGACALASQFTHPGEVFGELGRDSSEMASAAPAMPDGEWQAYGRTEHGDRYSPLRQITPQNAYRLEEAWRIRTGDLPTENDPVELTNQNTPLKVNGMLYACTAHSRLLALDPDTGAEIWRYDPQVKSPTGTFKGFAHMTCRGVSYYDENRYVSRDGSPAPKITDAGQAVAQACPRRLYLPTADARLIAINADNGKVCEGFANQGVIDLTTGIGPFTAGGYYSTSPAAITRDLVIIGGHVTDNESTNEPSGVIRAYDVHDGHLVWNWDSNNPDDTKPLAAGKMYSRNSANMWSIASVDEDLGMIYLPLGNQTPDQWGADRTPGAEKYSAGVVALDLATGKARWNYQFTHHDLWDMDVGSQPTLVHLKTDDGVKPAIIVPTKQGSLYVLDRRDGTPIVPIREIPTPQGAVEGDHTSPTQARSDLNLLGPELTEQAMWGATPFDQMLCRIQFRELRYEGQYTPPSEQGSLVYPGNVGVFNWGSVSVDPVRQLLFTSPNYMAFVSKMVPREQVAEGSKRESETSGVQPNTGAPYAVIMHPFMSPLGVPCQAPAWGYVAAIDLFTNKVVWKHKNGTTRDSTPLPIGLPVGVPSMGGSIVTAGGVGFLSGTLDQYLRAYDVNNGKELWKARLPAGGQATPMSYTGKDGKQYVLVTAGGHGSLGTKMGDYIIAYKLAE

SEQ ID NO: 5 - DNA, 1335 bp, Pseudomonas putida
  source 1..1335: mol_type = genomic DNA
atgttccaactgcctaaaacctgctacatcggcctggccctcagtgccttggcaacccccgccggcgccagcgaaatgttcgccagcgactccccttggatgctcggcgactggggtggaacccgcagcgaactgctggaaaagggctacgacttcacccttggctacaccggcgagatgggcagcaacctgcacggcggctacgaccacgaccgcaccgcgcgctacagcgaccagttcaccttcggcagccacctggacctggagaagatcctcggctggcacgacaccgaattccagctgaccgtcaccgagcgccacggcgacaacatcagcaacgaccgcatcaacgacccgcgtgtcggcggcttcacctccgcccaggaagtctggggccgcggcgaaacctggcgactgacgcagatgtggatcaagcagaagtacttcgacggtgcgctggatgtgaaattcggccgctttggtgaaggcgaggacttcaacagcttcccttgcgacttccagaacctggctttctgcggctcgcaggtgggcaactgggtgggtggcatctggtacaactggccagtcagccagtgggccctgcgcgtgcgctacaaccttacccccgagctgtacgcccaggtcggcgtcttcgagcagaacccttccaacctcgaatccggcaatggtttcaagctcagcggcagcggcacccagggtgcggtaatgccgttcgaactggtatggaccccacgtatccaaggcttgaaaggggaatatcgtgccggctactactacagtaatgccaaggcacaagatgttctcaaggacagcaacggtcagccggccgccctcagcggcgccgcctaccgcagcagttcgagcaagcacggcttgtggattggcgcccagcagcaggtcacctcgctggcgtccgaccagtcgcgcggcttgagcgtgttcgccaacgccacggtgcatgacaaaaagaccaatgccatcgacaactatgtgcaggcaggactggtattcaaagggcctttcgatgcccgcgccaaggacgacatcggtttcgccctggcccgcgtgcacgtcaaccctgcctatcgcaagaacgcccgcctggtcaaccaggccgccggcctctatgactacgacaacccgggcttcctgccagtgcaggacaccgagtacagcgccgagctgtattacggcattcacttggccgactggctcacggtacgccccaacctgcagtacatccgccacccgggcggggtgtcgcaggtcgatggcgccctgatcggcggcctgaagatccagagcagtttctaa

SEQ ID NO: 6 - protein, 444 aa, Pseudomonas putida
MFQLPKTCYIGLALSALATPAGASEMFASDSPWMLGDWGGTRSELLEKGYDFTLGYTGEMGSNLHGGYDHDRTARYSDQFTFGSHLDLEKILGWHDTEFQLTVTERHGDNISNDRINDPRVGGFTSAQEVWGRGETWRLTQMWIKQKYFDGALDVKFGRFGEGEDFNSFPCDFQNLAFCGSQVGNWVGGIWYNWPVSQWALRVRYNLTPELYAQVGVFEQNPSNLESGNGFKLSGSGTQGAVMPFELVWTPRIQGLKGEYRAGYYYSNAKAQDVLKDSNGQPAALSGAAYRSSSSKHGLWIGAQQQVTSLASDQSRGLSVFANATVHDKKTNAIDNYVQAGLVFKGPFDARAKDDIGFALARVHVNPAYRKNARLVNQAAGLYDYDNPGFLPVQDTEYSAELYYGIHLADWLTVRPNLQYIRHPGGVSQVDGALIGGLKIQSSF

SEQ ID NO: 7 - DNA, 966 bp, Enterobacter hormaechei
  source 1..966: mol_type = genomic DNA
atgacaaagtatgctttggtaggtgatgtaggcggcaccaacgcgcgccttgccctgtgcgatgtgaacagcggtgaaatttctcaggcgaaaacctattcagggctggattacccaagcctggaagcggtggttcgcgtctatctggaagagcataaagtgagcgttgaagacggttgtatcgccatcgcctgtccgatcaccggcgactgggtggcgatgaccaaccacacgtgggccttctcaattgccgaaatgcggaaaaacctcggcttctcgcaccttgaaattattaacgacttcactgcggtttccatggctatcccaatgcttaaaccagagcatctgattcagttcggcggtacggccccggttgaaggtaaaccgattgccgtttacggcgcgggcaccggcctgggggtggcgcacctggttcacgtcgacaaacgctgggtcagcctgccgggcgagggcggtcatgtggattttgcgccgaacagcgaagaagagggcattattctagaggagctacgcgccgagattggccacgtgtcggccgagcgggtgctttctggtccgggcctggtgaacctgtatcgggcgattgtaaaatcagacggtcgtctgccggaaaatctgcaaccgaaagatgtgaccgaacgcgcgctggccgacagctgtattgactgccgccgtgctttgtcgctgttctgcgtcatcatggggcgcttcggcggcaacctggcgctgaacctcggcacctttggcggggtctatattgcgggcgggatcgtgccgcgcttcctcgatttctttaccgcctccggcttccgtggcggcttcgaagacaaaggccgcttcagaagctatgtccaggacattcctgtttacctgattgtccacgataacccaggcttactgggctccggcgcgcatctgcgtcaggtactcggtcagattctctga

SEQ ID NO: 8 - protein, 321 aa, Enterobacter hormaechei
MTKYALVGDVGGTNARLALCDVNSGEISQAKTYSGLDYPSLEAVVRVYLEEHKVSVEDGCIAIACPITGDWVAMTNHTWAFSIAEMRKNLGFSHLEIINDFTAVSMAIPMLKPEHLIQFGGTAPVEGKPIAVYGAGTGLGVAHLVHVDKRWVSLPGEGGHVDFAPNSEEEGIILEELRAEIGHVSAERVLSGPGLVNLYRAIVKSDGRLPENLQPKDVTERALADSCIDCRRALSLFCVIMGRFGGNLALNLGTFGGVYIAGGIVPRFLDFFTASGFRGGFEDKGRFRSYVQDIPVYLIVHDNPGLLGSGAHLRQVLGQIL

SEQ ID NO: 9 - DNA, 2391 bp, Enterobacter hormaechei
  source 1..2391: mol_type = genomic DNA
atggctgaaacaaaaactaaacagccgcgtctactggtgacattaacagccgcgtttgccgcattctgcgcgctgtatttgttaatcggtggcgtatggctggtcgcgattggcggctcctggtactacccgattgcgggtctggttatggttggcgtaaccgtcctgcttttacgcagaaaacaatctgctctgtggctgtacgcagcgttactgctcgcaaccatgatctggggcgtctgggaagtcgggttcgacttctgggcgttgacgccgcgcagcgacattctggtcttcttcggtatctggctgatcctgccgtttgtctggcgtcgtctgatcgtcccttccagtggcgccgtggccagtctggtcgttgcccttctgattagtggcggcatcctgacctgggccggttttaacgacccgcaggagatcaacggtacgctgaacgcggaatccacgccggctgcggctatttcgcaggtggcggacggtgactggcctgcttatggtcgtaaccaggaaggtcaacgctactccccgctgaagcagatcaacgcggacaacgtgaaaaacctgaaggaagcctgggtattccgtaccggcgacctgaagatgccaaacgatccgggtgagctgaccaacgaagtgaccccgattaaagtgggcaacatgctctacctgtgtacggcgcaccagcgtctgttcgcgctcgacgcggctaccggtaaagagaaatggcactttgatccacagctgaactccaacccgtcgttccagcacattacctgccgcggcgtctcttaccacgaagcgcgcgccgataatgccagcccggaagtcattgccgactgtcctcgccgcattatgctgccagtgaacgatggccgcctgtttgccattaacgctgagaccggcaagctgtgcgaaaccttcgccaataaaggcattctgaatcttcagaccaacatgccggacaccacgccgggcctgtatgagccgacctccccgccaatcatcaccgataaaaccatcgtgattgccggttcggtaacggataacttctctacccgtgaaacctccggcgttatccgtggtttcgacgtgaacaccggtaaactgctgtgggccttcgacccgggcgcgaaagatcctaacgcgatcccatcggatgaacacacctttacctttaactcaccgaactcctgggcaccggcagcgtatgacgcgaagctggacctggtctacctgccgatgggcgtgaccacgccggatatctggggcggtaaccgtacgccggagcaggaacgttacgccagctccatcgtggcgctgaacgcaacaaccggtaaactggcatggagctaccagaccgttcaccacgatctgtgggatatggatatgccgtcccagccgacgctggcggacattaccgttaacggtaaaaccgtaccggtgatttacgccccggcgaaaaccggtaacatcttcgtgcttgatcgtagcaacggtaagctggttgttcctgcgccggaaaaaccggttccgcagggtgcggccaaaggcgactacgtcaccaaaacgcagccgttctctgacctgagcttccgtccgaagaaagatctcagcggtgcagacatgtggggtgccaccatgtttgaccagctggtatgccgcgtgatgttccaccagctgcgctacgaagggattttcaccccgccgtcagagcagggtacgctggtcttcccgggtaacctggggatgttcgaatggggtgggatctccgttgaccctaaccgtcaggtcgccatcgctaacccaatggcgctgccgttcgtttcccgcctgatccctcgtggtccgggtaacccaatggagcagccgaaagatgcgaaaggcagcggtaccgaagccggtatccagccacagtatggcgttccttatggtgtgaccctgaatccgttcctgtctccgtttggtctgccgtgtaaacagcctgcctggggttacatctccggtctggatctgaagaccaacaagatcgtctggaagaaacgtattggtacgccacaggacagcatgccgttcccgatgcctgttccagtgccgttcaatatgggtatgccaatgctgggtggtccaatctctaccgccggtaacgtgctgttcatcgcggcaaccgcagataactacctgcgcgcgtacaacatgaccaacggtgagaaactgtggcaaggccgtctgccagccggtggacaggcaacgccgatgacctatgaagtgaatggcaagcagtacgttgtcatctctgcgggtggtcacggttcgtttggcacgaagatgggcgactatattgtcgcgtatgcactgcctgacgacgctaagtaa

SEQ ID NO: 10 - protein, 796 aa, Enterobacter hormaechei
MAETKTKQPRLLVTLTAAFAAFCALYLLIGGVWLVAIGGSWYYPIAGLVMVGVTVLLLRRKQSALWLYAALLLATMIWGVWEVGFDFWALTPRSDILVFFGIWLILPFVWRRLIVPSSGAVASLVVALLISGGILTWAGFNDPQEINGTLNAESTPAAAISQVADGDWPAYGRNQEGQRYSPLKQINADNVKNLKEAWVFRTGDLKMPNDPGELTNEVTPIKVGNMLYLCTAHQRLFALDAATGKEKWHFDPQLNSNPSFQHITCRGVSYHEARADNASPEVIADCPRRIMLPVNDGRLFAINAETGKLCETFANKGILNLQTNMPDTTPGLYEPTSPPIITDKTIVIAGSVTDNFSTRETSGVIRGFDVNTGKLLWAFDPGAKDPNAIPSDEHTFTFNSPNSWAPAAYDAKLDLVYLPMGVTTPDIWGGNRTPEQERYASSIVALNATTGKLAWSYQTVHHDLWDMDMPSQPTLADITVNGKTVPVIYAPAKTGNIFVLDRSNGKLVVPAPEKPVPQGAAKGDYVTKTQPFSDLSFRPKKDLSGADMWGATMFDQLVCRVMFHQLRYEGIFTPPSEQGTLVFPGNLGMFEWGGISVDPNRQVAIANPMALPFVSRLIPRGPGNPMEQPKDAKGSGTEAGIQPQYGVPYGVTLNPFLSPFGLPCKQPAWGYISGLDLKTNKIVWKKRIGTPQDSMPFPMPVPVPFNMGMPMLGGPISTAGNVLFIAATADNYLRAYNMTNGEKLWQGRLPAGGQATPMTYEVNGKQYVVISAGGHGSFGTKMGDYIVAYALPDDAK

SEQ ID NO: 11 - DNA, 8282 bp, Cupriavidus basilensis
  source 1..8282: mol_type = genomic DNA
atggagcggctcgaggacgccgcgatcctcaccggccgtggccgctatggcgacgacctgggcgtgaagccgggcaccctgcacgcggccatcgtgcgttccccgcatgcacatgccgaactcggcaccatcgatgccaccgccgcgcttgccgcgccgggcgtgcatgccgtgctgaccggcgccgacctggcagcctggtcgcgtcccttcgtggtcgccgtgaagtcgccaatggagcaatgggcgctggccatggaccgcgtgcgctatgtgggcgagccggtggccgtggtaatggccgaaagccgtgccctggccgaagacgcgctcgacctggtgcgggtgaattaccgcgtgttgccgccggtggtatcgatcgaggctgcgctggccgacgatgcgcccatcctgcatcccggcgtaggcgccaatgtggtgagcgaccgccacttccgctacggcgagcctgaagccgcctttgccgccgcgccgcaccgggtcacgctgaccgcgcactatccgcgcaacacctgcacgccgatcgaatgcggcgtggtgattgccgagttcctgcccggtgacgaaggctacgacgtcacctccaatttcatggggccgttctcgctgcatgcggtgatggcgatggcgctcaaggtgccggccaaccggctgcgccacaaggccccgcgcgattccggcggcagctttggcgtcaagcaggcggtgtttccttacgcggtgctgatgtgcctggcgtcccgcaaggccggcgcgccggtcaagtgggtggaggaccgcctcgaacatctcagcgcggccacctccgccaccgcgcggctgtccacgctggaagccgcggtggagtcagacggccgcatcaaagcgctggcctatgaccagatcgaagactgcggcggctatctgcgcgcgccggagcccgccaccttctaccgcatgcacggctgcctgactggcgcctacgacatccccaacctgctggtgcgcaaccgcgtggtgatgaccaacaagacgcccaccggcctggtgcggggctttggcggcccgcaggtgtattttgcgctggagcggctggtgcatcgcatcgcgacacaactcgggctcgatccactcgatgtgtatcgccgcaactttgtcgccgccgatgcctttccctatcgcgccgcggcgggcgcgttgctggactccggcaactaccagctggcgctggcgcgcgcgctggaagaaggcggctactacgagctgacgtgccgccgcgaagtcgcacgcgccgagggccggctctacggcatcggctttgccgccatcgtcgagccgtcggtatcgaacatgggctatatcaccaccgccatgcccgccgaggcgcgcaagaaagcggggccgaagaacggcgccatcgccagcgccacggtcagcgtcgacctgctcggcggcgtggtggtcaccattgcctcgacgccggccgggcagggccatatgacagtgtgcgcgcaggtggtggccgatgtgctcggcgtgaatccggccgacgtggtcgtaaacgtcgagttcgatacgcacaaggatgcatggtcggtcgccgccggcaactactccagccgtttcgccggcgccgtggccggcaccgtgcacctggccgccgagcgggtgcgcgacaagctggcgcgcatcgtggcgccgcagttcggctgcacgcccgccgaggtggtgttcgaagacggccgcattgcccgcaaaggcgcgcccgaatccggcctgcccttcacgcgtgtggccagcaatgcgccgcactggtcaccgcagcagttgccagcgggagaggagccgggcctgcgcgagacggtgttctggtcgccgcccaacctggaagcgccggatgagaacgaccgcatcaacacctcggccgcctacggctttgccttcgatatgtgcggcgtggagatcgaccgcgccacggggcgcgtgcgcatcgaccgctacgtgaccgcgcacgacgccggcacgctgctcaacccggcgctggccgatggccagatccgcggcgccttcgcccaagggctgggcgcggcgctgatggaggagttccgctatggcgccgacggcagcttccagtccggcacgctggccgactacctgatgccgaccacctgcgaggtgccggacccggtgatcgtgcacctggaaacgcccagccccttcacgccgctcggcgccaagggcctgggcgagggcaacaacatgagcacgccgccctgcatcgccaacgcggtggccgacgcgctcggcgagcaggacattcgcctgccgctgaccccggccaaggtgatggccatggtgggcttcgacgatccgccgccgtcgcgccccgagctgctcgaagcgatgcgcgaggccgccgtgcccgcggcccgcaagggcagcgcaaaggcgctcaccgcgcgcggctcggtggatctggatgccacgcccgaagccatcttcgccgtgctgatggacccgcaggccctggccaaggtagtgccaggctgccacgcgctggagcgcaccgccgagaaccactaccgcgccgatgtgacggtgggggttggcatgatcaaggcccgcttcgaggcggagatcgcactgtcggacctcgatcccccgcgccggctgcggctggcaggcgcaggcatgtcctcgctgggcagtgcgcgcggcgccgggctggtggagctggtcccgcatggcagcggcacgcgcctgagttacgactacgaggccgaggtatccggcaaggtcgctgccgtgggcggccgcatgctggagggcgccgccaaggtggtgctgcgccagttgtttgaatcgctcggccgccaggccgccggcaagccggtacggccgcaaggctggcttgcccgactgctggcccgcttgggagtacgccgatgaaaccgtctgctttcgattacctgcgcgccgagaccacgcagcacgcgctcgaggcgctggcccgtggcggcgagggcgcgcgcgtgctggccggcggccagtcgctgatggcggtgctcaatatgcgcctggcgcagccgcaactgctgatcgatatctcgcgcaccgtcgagctggacacggtgcgggtggaagacgcgcacctggtggtgggtgccgcggccacgcagggcagcgtcgaatggcgccgctcgctggccgacgaggtgccgctgctggccatggcctttccgcatatctcgcatttccagatccggaatcgcggcaccgtgtgcggctcggtcgcccatgccgacccgagcgcggaattgcccctggtgctgaccgcgctgggcggcgaggtggtgctgcgttcagcccgccgccgccgcgtgctgcctgcggccagcttcttccagggcatgttgatgacggcgcgcgagcccgacgagctggtggaggccgtgcgctttccgctgcggcgccccggggcgcgctacggctttgccgaattctccgcgcgccacggcgattttgcgctggtggcctgcgcggccaccgtgaccgatgacgccatcgcgctggcggttggcggcgtggcggacaggccggtgctggaaacctggccgcgcctgcagggcaaggacctggagcaggccatcaacgatttcagttggaaactgggcgcgcaggacgacgcccatatcagcgcgcagtaccgccggcacctggtgcggcaactgagcatgcgtgtgatcgaggaggcaaaatgagcaagaccaacaagggcgccgtttccggcaagcccgccgaggtaatgcagcgccaggagcaacgccgcatcaccctgacgctgaacggccgcgagcgcagcggccattgcgagccgcgcgagctgctgtcggacttcctgcgccacgagctcggcgccaccggcacccatgtgggttgcgagcacggcgtctgcggcgcatgcacggtacgcgttgacggcgttgccgcgcgctcgtgcctgatgctggcggtgcaggccgagcaccgggccatcgataccgtcgaagggctggcgccggccgagggactgggcgacctgcaagaagccttccgccgccaccacgcgctgcagtgcggcttctgcaccgcaggaatcctgatgtcgtgcgcggactacctggagcgcgtgccggaacccagcgaggcgcaggtgcgcgacatgctgtccggccacctgtgccgctgtacgggctacacccccattgtggccgccgtgctcgacgtagccgcgatccgtgcacgggctcgtcatgctgctgccggcgtagatacccaggaggcctgcaatgcttgatctaggccgcaccttcctgcaaagcgtggagcgcagcccgcacacgcccgccattgtcgacggcgacctgatgctcacctatgcgcaatggtacgagcgcatccggtgcgtggcgtccggcctgcgcgagatcggcctcgcgccgggcgatcgcctgctggccgtgttgcaaaaccgctgggaaatggccacgctgcactgggcctgccagttcgccggcatcgtgatggtgccgctgaactggcgcgccaagccggaggagctcgattactgtgtgcaggatgccggcgtcaaggcgctggtgttcgagccggtcagcgccgatgcggtgctgggcagccccgcggcgcaggccgtgccctgcattgcgctggactgcgcggctggcggctcgatgtccttcgcttcgctgctggacagcgtcgcgctgcatggcgacccggtggcacaagcgagcgatgtctcgctgatgctctacacctcgggcaccaccggcaagcccaagggcgtgccgcgccgccaccagcacgagcgcgccgcggcgctggcgcacgtggcgcagaacctgtatcgccatggtgagcgcacccttggcgtgatgccgctctaccacaccatgggcgtgcgctcgctgctggcaatggcgctggtggatggcctgttcgtctgcgtgcggcgctggaacgccgggcaggcgctcgaggagatcaacacccaccgaatcagctgcctgtacctggtgccgacgctgtaccacgacctgctggccgatccggggttcgatgcctgcatcgtgcgcagcgtgagcaagctcggctttgccggcgcctcgatgaacgacggcctgctgcgccgccttgcgctggccttcgagccggagctgttcgtgaaccactacggctcatccgaggtgtacaccttcagcgtggaccagcgcgccacccgcaagcccggcagcgccgggcgcgccggcatcaatacgcgcctgcgcgtggtgcgcctggatgcccgctcacccgacgatctggcggctaccggcgaggaaggccagatcatcgccgacctgcgcggcgacgaggccttcgagggctactggaaccgcgacgacgccaacgccaaatcgctgcgcgatggctggtactttaccggcgacaccggctacttcgatgccgagggcgatctcttcgtcagcggccgggtggacgacatgatcatcagcggcggcgagaacatctccccggtcgatatcgaatcggtgctgtcgctgcatccggcggtcgatgaggtggcggtggccggcgtgccggatccgcgctggggccagaaggtggtggctttcgtcaagccgcgcggcaacatcgacgcgcaagccctggatacctactgccgcggctctgacctggtcaatttcaagcgcccgcgcgactacgtcttcgtggaggagattcccaagtcgccggtcggcaagatcctgcgccgcaagttgtccgccggcgaatacgcgctggccccgcattccatgagccccgaccctaataccaaccccaaccccaaccaggctgcggacgccgcgcctgtcgataccatcaaggagtaagacatgacccaggcaaccgagatgatccatcccgaccagcagcggctccagcaactcgacggcttctccgtggagatcgatgccgggcgcgagcgtgcggatatcatcctgcaccgtccaccctacaacgtgatcgccatggcggcgcgcgaccagttgcgtgccgtcattgaagcgctggatgccgacgatcgcgtgcgcgtgatcgtgctgcgttcgcaaggcgagcatttttccagcggcggcgatatcaagggcttcctggaggcatcgcccgagcatgtctcgcaactggcctggaatgtggcggcgccggcgcgctgcagcaagccggtgattgccgccaaccgcggctactgctttggcgtgggcttcgagctgtcgctggcgtgcgacttccgcatcgccaccgagaccacgcagtacgcgctgccggaacagaagctcggccagatccccggctcgggcggctcggcgcgcctgcagaagatggtgggcatcggccgcaccaaggacatcgtgatgcgctcgcgccgcatctcgggcaagcaggcctatgagtggggcatcgccgtggaatgcgtggcagacgccgagctggaggccgccaccgatgcgctggtcgacgagctgcgcggcttctcgccgctggcgcagcgcaccgccaagaagctgctcaacgacaccgaggacgcgccgctgtcgattgccatcgagctggaagggcattgctatagccgcctgcgcagctcggacgatttccgcgaaggcgtggaagccttccacggcaagcgcaagccggcgttccgcggcagctgatggaaatcgcgggcgcgccggcaaggccgctcgcatcccggatgaactgaccggataacgatgcccgctgccacggggcaagcatcgcggggggagcgcttgcctggcggcaaggcaccgcccgcgcgccacccgctgccgtcatgatccaagcagagatccaaatcaaaggagacaacaatggaagccgtagcaaagaagagtgcagcgacgatcagcgaggcgctgccagcggcgagcaatcgccaggtgtttggtgccgtggcggcgtcgtgcatgggatgggcgctggacctgttcgacctgttcatcctgctgttcgtggcgcccgtgatcggcaggctgtttttcccgtcggagcacgccatgctgtcgctggcggcggtgtatgcgtcgtttgccgtgacgctgctgatgcggccgctcggctcggcgatcttcggctcttatgccgaccgccatggccgcaagggggcgatggtagttgccgtcactggcgttggcttgtccacggcggcgttcggcctactgccgacggtgggtcaggtggggctgcttgcgccagccttgtttatcctgctgcggctggtgcagggcatcttcgtgggtggcgtggtggcatccacccacactatcggtaccgaatcggtgcccccgtcctggcgcggcgccgtttccgggctggtcggtggcggtggcgcgggtatcggggcgctgctggcttccattacctacatggcgatgaccgcgctgtttccgggggaagcgttcgacgcctggggttggcgctgcatgttcttctccggcatcatcagctcggtgctcggcctgttcatcttcaactcgctggaggagtctccgctgtggaagcagttgcaggcggccaaggggcacgccgcgccggttgagaacccgctgcgcgtgatcttctcgcgccagtaccgtggcgtcctcttcgtcaacatcctgctcaccgtgggcggtggcagcgcctactacctgacctccggctatctgccgaccttcctcaaggtggtggtgaaggcatcggccggcgaggctgccgccatcctgatggccagcagtctgggtgtgatcgtggcgtcgatccttgccggccacctcagtacgatgatcggccgcaagcgagccttcctgttgatcggcgcgctgaacgtggtgctgctgccgctgctctaccagtggatgccggcggcgccggacaccaccacgctcggcctgtatgccgtggtgctgtccatgctgggctgcagcggcttcgccccgatcctcattttcctgaacgaacggttccccaccagcatccgtgccacggggaccggcctgtcatggaatatcggctttgccgtcggtggcatgatgccgacctttgcttcgctgtgcgccagcacccctgccgaactgcccatggtgctgggcatcttcctggcggttgtcaccatcatctacctggtgggtgcgttcatcgttccggagacggcagggcgccttggcgacaatggagcgtag

SEQ ID NO: 12 - DNA, 2949 bp, Cupriavidus basilensis
  source 1..2949: mol_type = genomic DNA
atggagcggctcgaggacgccgcgatcctcaccggccgtggccgctatggcgacgacctgggcgtgaagccgggcaccctgcacgcggccatcgtgcgttccccgcatgcacatgccgaactcggcaccatcgatgccaccgccgcgcttgccgcgccgggcgtgcatgccgtgctgaccggcgccgacctggcagcctggtcgcgtcccttcgtggtcgccgtgaagtcgccaatggagcaatgggcgctggccatggaccgcgtgcgctatgtgggcgagccggtggccgtggtaatggccgaaagccgtgccctggccgaagacgcgctcgacctggtgcgggtgaattaccgcgtgttgccgccggtggtatcgatcgaggctgcgctggccgacgatgcgcccatcctgcatcccggcgtaggcgccaatgtggtgagcgaccgccacttccgctacggcgagcctgaagccgcctttgccgccgcgccgcaccgggtcacgctgaccgcgcactatccgcgcaacacctgcacgccgatcgaatgcggcgtggtgattgccgagttcctgcccggtgacgaaggctacgacgtcacctccaatttcatggggccgttctcgctgcatgcggtgatggcgatggcgctcaaggtgccggccaaccggctgcgccacaaggccccgcgcgattccggcggcagctttggcgtcaagcaggcggtgtttccttacgcggtgctgatgtgcctggcgtcccgcaaggccggcgcgccggtcaagtgggtggaggaccgcctcgaacatctcagcgcggccacctccgccaccgcgcggctgtccacgctggaagccgcggtggagtcagacggccgcatcaaagcgctggcctatgaccagatcgaagactgcggcggctatctgcgcgcgccggagcccgccaccttctaccgcatgcacggctgcctgactggcgcctacgacatccccaacctgctggtgcgcaaccgcgtggtgatgaccaacaagacgcccaccggcctggtgcggggctttggcggcccgcaggtgtattttgcgctggagcggctggtgcatcgcatcgcgacacaactcgggctcgatccactcgatgtgtatcgccgcaactttgtcgccgccgatgcctttccctatcgcgccgcggcgggcgcgttgctggactccggcaactaccagctggcgctggcgcgcgcgctggaagaaggcggctactacgagctgacgtgccgccgcgaagtcgcacgcgccgagggccggctctacggcatcggctttgccgccatcgtcgagccgtcggtatcgaacatgggctatatcaccaccgccatgcccgccgaggcgcgcaagaaagcggggccgaagaacggcgccatcgccagcgccacggtcagcgtcgacctgctcggcggcgtggtggtcaccattgcctcgacgccggccgggcagggccatatgacagtgtgcgcgcaggtggtggccgatgtgctcggcgtgaatccggccgacgtggtcgtaaacgtcgagttcgatacgcacaaggatgcatggtcggtcgccgccggcaactactccagccgtttcgccggcgccgtggccggcaccgtgcacctggccgccgagcgggtgcgcgacaagctggcgcgcatcgtggcgccgcagttcggctgcacgcccgccgaggtggtgttcgaagacggccgcattgcccgcaaaggcgcgcccgaatccggcctgcccttcacgcgtgtggccagcaatgcgccgcactggtcaccgcagcagttgccagcgggagaggagccgggcctgcgcgagacggtgttctggtcgccgcccaacctggaagcgccggatgagaacgaccgcatcaacacctcggccgcctacggctttgccttcgatatgtgcggcgtggagatcgaccgcgccacggggcgcgtgcgcatcgaccgctacgtgaccgcgcacgacgccggcacgctgctcaacccggcgctggccgatggccagatccgcggcgccttcgcccaagggctgggcgcggcgctgatggaggagttccgctatggcgccgacggcagcttccagtccggcacgctggccgactacctgatgccgaccacctgcgaggtgccggacccggtgatcgtgcacctggaaacgcccagccccttcacgccgctcggcgccaagggcctgggcgagggcaacaacatgagcacgccgccctgcatcgccaacgcggtggccgacgcgctcggcgagcaggacattcgcctgccgctgaccccggccaaggtgatggccatggtgggcttcgacgatccgccgccgtcgcgccccgagctgctcgaagcgatgcgcgaggccgccgtgcccgcggcccgcaagggcagcgcaaaggcgctcaccgcgcgcggctcggtggatctggatgccacgcccgaagccatcttcgccgtgctgatggacccgcaggccctggccaaggtagtgccaggctgccacgcgctggagcgcaccgccgagaaccactaccgcgccgatgtgacggtgggggttggcatgatcaaggcccgcttcgaggcggagatcgcactgtcggacctcgatcccccgcgccggctgcggctggcaggcgcaggcatgtcctcgctgggcagtgcgcgcggcgccgggctggtggagctggtcccgcatggcagcggcacgcgcctgagttacgactacgaggccgaggtatccggcaaggtcgctgccgtgggcggccgcatgctggagggcgccgccaaggtggtgctgcgccagttgtttgaatcgctcggccgccaggccgccggcaagccggtacggccgcaaggctggcttgcccgactgctggcccgcttgggagtacgccgatga

SEQ ID NO: 13 - protein, 982 aa, Cupriavidus basilensis
MERLEDAAILTGRGRYGDDLGVKPGTLHAAIVRSPHAHAELGTIDATAALAAPGVHAVLTGADLAAWSRPFVVAVKSPMEQWALAMDRVRYVGEPVAVVMAESRALAEDALDLVRVNYRVLPPVVSIEAALADDAPILHPGVGANVVSDRHFRYGEPEAAFAAAPHRVTLTAHYPRNTCTPIECGVVIAEFLPGDEGYDVTSNFMGPFSLHAVMAMALKVPANRLRHKAPRDSGGSFGVKQAVFPYAVLMCLASRKAGAPVKWVEDRLEHLSAATSATARLSTLEAAVESDGRIKALAYDQIEDCGGYLRAPEPATFYRMHGCLTGAYDIPNLLVRNRVVMTNKTPTGLVRGFGGPQVYFALERLVHRIATQLGLDPLDVYRRNFVAADAFPYRAAAGALLDSGNYQLALARALEEGGYYELTCRREVARAEGRLYGIGFAAIVEPSVSNMGYITTAMPAEARKKAGPKNGAIASATVSVDLLGGVVVTIASTPAGQGHMTVCAQVVADVLGVNPADVVVNVEFDTHKDAWSVAAGNYSSRFAGAVAGTVHLAAERVRDKLARIVAPQFGCTPAEVVFEDGRIARKGAPESGLPFTRVASNAPHWSPQQLPAGEEPGLRETVFWSPPNLEAPDENDRINTSAAYGFAFDMCGVEIDRATGRVRIDRYVTAHDAGTLLNPALADGQIRGAFAQGLGAALMEEFRYGADGSFQSGTLADYLMPTTCEVPDPVIVHLETPSPFTPLGAKGLGEGNNMSTPPCIANAVADALGEQDIRLPLTPAKVMAMVGFDDPPPSRPELLEAMREAAVPAARKGSAKALTARGSVDLDATPEAIFAVLMDPQALAKVVPGCHALERTAENHYRADVTVGVGMIKARFEAEIALSDLDPPRRLRLAGAGMSSLGSARGAGLVELVPHGSGTRLSYDYEAEVSGKVAAVGGRMLEGAAKVVLRQLFESLGRQAAGKPVRPQGWLARLLARLGVRR

SEQ ID NO: 14 - DNA, 816 bp, Cupriavidus basilensis
  source 1..816: mol_type = genomic DNA
atgaaaccgtctgctttcgattacctgcgcgccgagaccacgcagcacgcgctcgaggcgctggcccgtggcggcgagggcgcgcgcgtgctggccggcggccagtcgctgatggcggtgctcaatatgcgcctggcgcagccgcaactgctgatcgatatctcgcgcaccgtcgagctggacacggtgcgggtggaagacgcgcacctggtggtgggtgccgcggccacgcagggcagcgtcgaatggcgccgctcgctggccgacgaggtgccgctgctggccatggcctttccgcatatctcgcatttccagatccggaatcgcggcaccgtgtgcggctcggtcgcccatgccgacccgagcgcggaattgcccctggtgctgaccgcgctgggcggcgaggtggtgctgcgttcagcccgccgccgccgcgtgctgcctgcggccagcttcttccagggcatgttgatgacggcgcgcgagcccgacgagctggtggaggccgtgcgctttccgctgcggcgccccggggcgcgctacggctttgccgaattctccgcgcgccacggcgattttgcgctggtggcctgcgcggccaccgtgaccgatgacgccatcgcgctggcggttggcggcgtggcggacaggccggtgctggaaacctggccgcgcctgcagggcaaggacctggagcaggccatcaacgatttcagttggaaactgggcgcgcaggacgacgcccatatcagcgcgcagtaccgccggcacctggtgcggcaactgagcatgcgtgtgatcgaggaggcaaaatga

SEQ ID NO: 15 - protein, 271 aa, Cupriavidus basilensis
MKPSAFDYLRAETTQHALEALARGGEGARVLAGGQSLMAVLNMRLAQPQLLIDISRTVELDTVRVEDAHLVVGAAATQGSVEWRRSLADEVPLLAMAFPHISHFQIRNRGTVCGSVAHADPSAELPLVLTALGGEVVLRSARRRRVLPAASFFQGMLMTAREPDELVEAVRFPLRRPGARYGFAEFSARHGDFALVACAATVTDDAIALAVGGVADRPVLETWPRLQGKDLEQAINDFSWKLGAQDDAHISAQYRRHLVRQLSMRVIEEAK

SEQ ID NO: 16 - DNA, 579 bp, Cupriavidus basilensis
  source 1..579: mol_type = genomic DNA
atgagcaagaccaacaagggcgccgtttccggcaagcccgccgaggtaatgcagcgccaggagcaacgccgcatcaccctgacgctgaacggccgcgagcgcagcggccattgcgagccgcgcgagctgctgtcggacttcctgcgccacgagctcggcgccaccggcacccatgtgggttgcgagcacggcgtctgcggcgcatgcacggtacgcgttgacggcgttgccgcgcgctcgtgcctgatgctggcggtgcaggccgagcaccgggccatcgataccgtcgaagggctggcgccggccgagggactgggcgacctgcaagaagccttccgccgccaccacgcgctgcagtgcggcttctgcaccgcaggaatcctgatgtcgtgcgcggactacctggagcgcgtgccggaacccagcgaggcgcaggtgcgcgacatgctgtccggccacctgtgccgctgtacgggctacacccccattgtggccgccgtgctcgacgtagccgcgatccgtgcacgggctcgtcatgctgctgccggcgtagatacccaggaggcctgcaatgcttga

SEQ ID NO: 17 - protein, 192 aa, Cupriavidus basilensis
MSKTNKGAVSGKPAEVMQRQEQRRITLTLNGRERSGHCEPRELLSDFLRHELGATGTHVGCEHGVCGACTVRVDGVAARSCLMLAVQAEHRAIDTVEGLAPAEGLGDLQEAFRRHHALQCGFCTAGILMSCADYLERVPEPSEAQVRDMLSGHLCRCTGYTPIVAAVLDVAAIRARARHAAAGVDTQEACNA

SEQ ID NO: 18 - DNA, 1605 bp, Cupriavidus basilensis
  source 1..1605: mol_type = genomic DNA
atgcttgatctaggccgcaccttcctgcaaagcgtggagcgcagcccgcacacgcccgccattgtcgacggcgacctgatgctcacctatgcgcaatggtacgagcgcatccggtgcgtggcgtccggcctgcgcgagatcggcctcgcgccgggcgatcgcctgctggccgtgttgcaaaaccgctgggaaatggccacgctgcactgggcctgccagttcgccggcatcgtgatggtgccgctgaactggcgcgccaagccggaggagctcgattactgtgtgcaggatgccggcgtcaaggcgctggtgttcgagccggtcagcgccgatgcggtgctgggcagccccgcggcgcaggccgtgccctgcattgcgctggactgcgcggctggcggctcgatgtccttcgcttcgctgctggacagcgtcgcgctgcatggcgacccggtggcacaagcgagcgatgtctcgctgatgctctacacctcgggcaccaccggcaagcccaagggcgtgccgcgccgccaccagcacgagcgcgccgcggcgctggcgcacgtggcgcagaacctgtatcgccatggtgagcgcacccttggcgtgatgccgctctaccacaccatgggcgtgcgctcgctgctggcaatggcgctggtggatggcctgttcgtctgcgtgcggcgctggaacgccgggcaggcgctcgaggagatcaacacccaccgaatcagctgcctgtacctggtgccgacgctgtaccacgacctgctggccgatccggggttcgatgcctgcatcgtgcgcagcgtgagcaagctcggctttgccggcgcctcgatgaacgacggcctgctgcgccgccttgcgctggccttcgagccggagctgttcgtgaaccactacggctcatccgaggtgtacaccttcagcgtggaccagcgcgccacccgcaagcccggcagcgccgggcgcgccggcatcaatacgcgcctgcgcgtggtgcgcctggatgcccgctcacccgacgatctggcggctaccggcgaggaaggccagatcatcgccgacctgcgcggcgacgaggccttcgagggctactggaaccgcgacgacgccaacgccaaatcgctgcgcgatggctggtactttaccggcgacaccggctacttcgatgccgagggcgatctcttcgtcagcggccgggtggacgacatgatcatcagcggcggcgagaacatctccccggtcgatatcgaatcggtgctgtcgctgcatccggcggtcgatgaggtggcggtggccggcgtgccggatccgcgctggggccagaaggtggtggctttcgtcaagccgcgcggcaacatcgacgcgcaagccctggatacctactgccgcggctctgacctggtcaatttcaagcgcccgcgcgactacgtcttcgtggaggagattcccaagtcgccggtcggcaagatcctgcgccgcaagttgtccgccggcgaatacgcgctggccccgcattccatgagccccgaccctaataccaaccccaaccccaaccaggctgcggacgccgcgcctgtcgataccatcaaggagtaa

SEQ ID NO: 19 - protein, 534 aa, Cupriavidus basilensis
MLDLGRTFLQSVERSPHTPAIVDGDLMLTYAQWYERIRCVASGLREIGLAPGDRLLAVLQNRWEMATLHWACQFAGIVMVPLNWRAKPEELDYCVQDAGVKALVFEPVSADAVLGSPAAQAVPCIALDCAAGGSMSFASLLDSVALHGDPVAQASDVSLMLYTSGTTGKPKGVPRRHQHERAAALAHVAQNLYRHGERTLGVMPLYHTMGVRSLLAMALVDGLFVCVRRWNAGQALEEINTHRISCLYLVPTLYHDLLADPGFDACIVRSVSKLGFAGASMNDGLLRRLALAFEPELFVNHYGSSEVYTFSVDQRATRKPGSAGRAGINTRLRVVRLDARSPDDLAATGEEGQIIADLRGDEAFEGYWNRDDANAKSLRDGWYFTGDTGYFDAEGDLFVSGRVDDMIISGGENISPVDIESVLSLHPAVDEVAVAGVPDPRWGQKVVAFVKPRGNIDAQALDTYCRGSDLVNFKRPRDYVFVEEIPKSPVGKILRRKLSAGEYALAPHSMSPDPNTNPNPNQAADAAPVDTIKE

SEQ ID NO: 20 - DNA, 816 bp, Cupriavidus basilensis
  source 1..816: mol_type = genomic DNA
atgacccaggcaaccgagatgatccatcccgaccagcagcggctccagcaactcgacggcttctccgtggagatcgatgccgggcgcgagcgtgcggatatcatcctgcaccgtccaccctacaacgtgatcgccatggcggcgcgcgaccagttgcgtgccgtcattgaagcgctggatgccgacgatcgcgtgcgcgtgatcgtgctgcgttcgcaaggcgagcatttttccagcggcggcgatatcaagggcttcctggaggcatcgcccgagcatgtctcgcaactggcctggaatgtggcggcgccggcgcgctgcagcaagccggtgattgccgccaaccgcggctactgctttggcgtgggcttcgagctgtcgctggcgtgcgacttccgcatcgccaccgagaccacgcagtacgcgctgccggaacagaagctcggccagatccccggctcgggcggctcggcgcgcctgcagaagatggtgggcatcggccgcaccaaggacatcgtgatgcgctcgcgccgcatctcgggcaagcaggcctatgagtggggcatcgccgtggaatgcgtggcagacgccgagctggaggccgccaccgatgcgctggtcgacgagctgcgcggcttctcgccgctggcgcagcgcaccgccaagaagctgctcaacgacaccgaggacgcgccgctgtcgattgccatcgagctggaagggcattgctatagccgcctgcgcagctcggacgatttccgcgaaggcgtggaagccttccacggcaagcgcaagccggcgttccgcggcagctga

SEQ ID NO: 21 - protein, 271 aa, Cupriavidus basilensis
MTQATEMIHPDQQRLQQLDGFSVEIDAGRERADIILHRPPYNVIAMAARDQLRAVIEALDADDRVRVIVLRSQGEHFSSGGDIKGFLEASPEHVSQLAWNVAAPARCSKPVIAANRGYCFGVGFELSLACDFRIATETTQYALPEQKLGQIPGSGGSARLQKMVGIGRTKDIVMRSRRISGKQAYEWGIAVECVADAELEAATDALVDELRGFSPLAQRTAKKLLNDTEDAPLSIAIELEGHCYSRLRSSDDFREGVEAFHGKRKPAFRGS

SEQ ID NO: 22 - DNA, 1350 bp, Cupriavidus basilensis
  source 1..1350: mol_type = genomic DNA
atggaagccgtagcaaagaagagtgcagcgacgatcagcgaggcgctgccagcggcgagcaatcgccaggtgtttggtgccgtggcggcgtcgtgcatgggatgggcgctggacctgttcgacctgttcatcctgctgttcgtggcgcccgtgatcggcaggctgtttttcccgtcggagcacgccatgctgtcgctggcggcggtgtatgcgtcgtttgccgtgacgctgctgatgcggccgctcggctcggcgatcttcggctcttatgccgaccgccatggccgcaagggggcgatggtagttgccgtcactggcgttggcttgtccacggcggcgttcggcctactgccgacggtgggtcaggtggggctgcttgcgccagccttgtttatcctgctgcggctggtgcagggcatcttcgtgggtggcgtggtggcatccacccacactatcggtaccgaatcggtgcccccgtcctggcgcggcgccgtttccgggctggtcggtggcggtggcgcgggtatcggggcgctgctggcttccattacctacatggcgatgaccgcgctgtttccgggggaagcgttcgacgcctggggttggcgctgcatgttcttctccggcatcatcagctcggtgctcggcctgttcatcttcaactcgctggaggagtctccgctgtggaagcagttgcaggcggccaaggggcacgccgcgccggttgagaacccgctgcgcgtgatcttctcgcgccagtaccgtggcgtcctcttcgtcaacatcctgctcaccgtgggcggtggcagcgcctactacctgacctccggctatctgccgaccttcctcaaggtggtggtgaaggcatcggccggcgaggctgccgccatcctgatggccagcagtctgggtgtgatcgtggcgtcgatccttgccggccacctcagtacgatgatcggccgcaagcgagccttcctgttgatcggcgcgctgaacgtggtgctgctgccgctgctctaccagtggatgccggcggcgccggacaccaccacgctcggcctgtatgccgtggtgctgtccatgctgggctgcagcggcttcgccccgatcctcattttcctgaacgaacggttccccaccagcatccgtgccacggggaccggcctgtcatggaatatcggctttgccgtcggtggcatgatgccgacctttgcttcgctgtgcgccagcacccctgccgaactgcccatggtgctgggcatcttcctggcggttgtcaccatcatctacctggtgggtgcgttcatcgttccggagacggcagggcgccttggcgacaatggagcgtag

SEQ ID NO: 23 - protein, 449 aa, Cupriavidus basilensis
MEAVAKKSAATISEALPAASNRQVFGAVAASCMGWALDLFDLFILLFVAPVIGRLFFPSEHAMLSLAAVYASFAVTLLMRPLGSAIFGSYADRHGRKGAMVVAVTGVGLSTAAFGLLPTVGQVGLLAPALFILLRLVQGIFVGGVVASTHTIGTESVPPSWRGAVSGLVGGGGAGIGALLASITYMAMTALFPGEAFDAWGWRCMFFSGIISSVLGLFIFNSLEESPLWKQLQAAKGHAAPVENPLRVIFSRQYRGVLFVNILLTVGGGSAYYLTSGYLPTFLKVVVKASAGEAAAILMASSLGVIVASILAGHLSTMIGRKRAFLLIGALNVVLLPLLYQWMPAAPDTTTLGLYAVVLSMLGCSGFAPILIFLNERFPTSIRATGTGLSWNIGFAVGGMMPTFASLCASTPAELPMVLGIFLAVVTIIYLVGAFIVPETAGRLGDNGA

SEQ ID NO: 24 - DNA, 7206 bp, Cupriavidus basilensis
  source 1..7206: mol_type = genomic DNA
atgtccaggcaacccgccgaaaagacgaacagcgcgtcgcaagccgcgccattgcccgaagggccgctgaccctgcgcagctggctgcgccacctcggcaacacggaccggctggctgcaatcgacgagccggtggcactagagcacaccctcgcagccgtcgccaagcggctcgatggcgagcgcgcggcgttcttccgccggcctggcggccacgccgttccggtcgtgagcgggttcatgtcgcgccgcgcgtggatcgccgaggcgatgggcgtgcccgaggccggcttgctcgagcgcgtgcgcagcgccgccgcgcagcccttgccggtgagcgaggtggcccagggcgaggccgcatgccagcaggtcatccacctggacaaggtggacctgcgcaagctgttgcccatcccgacccacagcgagcatgacaacggcccctatatcactgcaggcctggccatcgcgcgcaacccgcgcaccggcgtgcagaacgtatcgatccaccgcatccaggtgcatgccgcggatcgcatggccatcctgatgctgccgcggcatctcgatgcgttctaccgtgcggcggaggaatgtggcgaggcgctgccgattgccattgtcatcggtgtcgatccgctcaccatgctggcttcgcaggccatcacgcctatcgaccatgacgagttggagatcgccggggcattgcacggtgcgccgctggaagtgatcaagtgccgcaccagcgatgtgcgtgtgccggccaatgccgagatcgtgatcgagggccggcttctgcccggcgagcgcgagatggaagggccctttggcgaatttcccaagtactacagcagcgccgagccgcgcgaggtcatccaggtcgacgccgtcacgcaccgtcatcgcccgatctatcacaccatcgtgccggcggagatggagcacctgctgctcggggcgattccgcgcgaggcgaccttgctggcgcatctgcagcgcagccatcccggggtgcaggatgtgcatctgtcggtgggcggcgtatgccggtaccacttgtacgtaaagctcgacaagaagcgcgagggcgaagcgaagaacgtcattctcgcggcgtttggcgcgcactacgacatcaagcaggtggtcgtggtggataccgatgtcgacgtccacgacccggccgaggtggaatgggccgtcgcgacccgcttccaggcagaccaggacctggtggtgatcgccggggcgcagggctcggtgctcgacccctccacgaccgtcgccgccaacctcgccggcatcgacaatcccgagccgcacctgcaaggcatctgcgccaagatggggctggacgcgacccgcccggtcaagtacgcggcgcatgtgttcacccgcgtgcggattcccggcgagtcaaccatcgatttgcaggcgctggtgtcggtcgacccatcgcactgggaatcgtatctcggcgaaggagcttgatgccatggtcagccagagacgcatcattgtcggtatatccggcgccagcggcgcggccatcggcgtcaatctcctcaaggcgatgcgcaacctggacggcgtcgagtcgcacctcatcgtgtcggcgtccggcatgctcaccgcgacccaggaactcggcatcaagcgcagcgaactcgaggcacttgccgacgtagtgcataacgtgcgcgatattggcgcagcggtggccagcggctctttcgtgaccgagggcatggtggtcgcgccgtgctcgatgaagacactagcgtcggtggccaacgggttttccgacaacctgctgacccgcgccgcggacgtggtgctcaaggagcggcggcgcctggtgctggtggcgcgcgaaaccccgctgaacctcgcgcacctgcgcaacatgctgcacgccaccgagatgggcgcgatcgtgatgccgcccgtgcccgccttctactcgcatccgaccagcatcgaggatgtggtcaatcacaccgtgggccgcatcctggacctgttccagatcgagcacgacacgctggtcagccgctggtcgggcctcgctcacgactttgccggctgacggccggcgctgcggaccggggcgctgatcccggccggactttgaatgctgaacaggcaggagacagacatgaaaacttggctcgcccatgccgggctcgctttgctgttgctcacggggcttgcccatgcgcaaggctatcccaccaagccgatccgcatcgtggtgccgtacccgcccggcggcttcaacgacacgctggcccgcatcgtcggcagcaggctcaccgcagcctggggccagcccgtggtggtggacaacaagcccggcgcgggcaccatcatcggcacctcgttcgtggccaaggccgcgcccgatggctacacgctgctggtggtgcagttccccttcggtgccaatccctggctctataagtcgctgccgtacgacaccctcaaggacttcacgccggtcatcctggcaggcgagtcgccgatgacccttgtggtgaccaacggatcgccgatacgctccgtggacgatcttgtcaaatccgccaagggcacgcccggcaagatcaactacggctcgtcgggcagcggctcgtccaaccaccttgccatggcgttgttcgagcgcagtgccggcatcacgctcgcccaggttccctacaagggcagcacgcccatgctgaccgatctggccggcgggcaggtcgaagtggccttcgacgcgttgccccatgtgttgcctttcgtgaggtccggcaaggtacgcgcacttgccgtcgccgaccggagccgctttgcgtcgcttgccacggtgcccaccatggccgagagcggcctgcccggctacgacgcgtcatcctggcacggcattgtcgcgccggccggcacgcccccggagattgtgcaaaagctgaacgcgcagatcaacgacgcgctgcgcacggcggatgtgcgcaagctcttccacgagcagggcgtgcgtcccgacggcggcagccctgccgacttctctgcgtttatcggcaaggaaatggcgaagtggaagcaggtggtacatgacgcggccatccccttgcaatgacgaagcaacgacgcctattgacaaggggaggccgcatggatacgccgagggagcgtttcgactacgtgattgttggcggcgggtccgccggttgcgtactggccaatcgcctgtcgcaggacccggccatccgcgtcgcgctgatcgaggggggcatcgatacgccgccggacgctgtgccggcggagatcctcgacagctatccgatgcccttgttctacggtgaccggtatatctggccatcgctgcaagcccgcgccgtggcagggggcaggtccaaggtctacgagcaggggcgcgtcatgggcggcggctccagcatcaacgtgcaggccgcaaaccgcgggctgccgcgcgactacgatgagtgggccgcgttgggcgcgtccggctggtcgtggcaggatgtgctgccgtacttccgccgccttgagcgcgatgtggattacggcaacagcccgctgcacggcagccacggaccggtgccgatccgccgcatcctgccgcaggcttggccgccgttctgcacggagtttgcgcaagcgatgggccgcagcggcctgtccgagctggccgaccagaacgcggagttcggcgatggctggtttccggccgccttctcgaacctggatgacaaacgggtttcgaccgccatcgcctatctcgacgcggatacgcgccggcgggtcaatctgcggatctatgccgagacaacggtgcgcaagctcgtcgtatccggccgggaagcgcgtggggtgatcgccatccgggccgatgggtcgcggctggcgctggacgccggggaggtcatcgtgtccgcgggcgccttgcagtcgcccgccatcctgatgcgcgcggggatcggcgacgctggcgcgctgcaggccctcggcatcgaggtcgtagccgaccggcccggcgttggccgcaatctccaggatcatccggcgctgacgttctgccagttcctcgcgccccagtaccgcatgccgctctcgcgccggcgcgccagcatgacggcggcgcgcttctcatcggaggtgccaggcggcgaggcgtcggacatgtacctgtccagttccacgcgggcgggctggcatgcactcggtaatcggctcggcctcttcttcctgtggtgcaatcggccattctcgcgcgggcaggttcgccttgcgggagcccagccggatatgccgcccgtggtggagctcaacctgctcgacgacgagcgggatctgcggcgcatggtggccggcgtacgcaagttggtgcagatcgtgggtgcgtcggccttgcatcagcatccgggtgatttcttccccgctacgttttcgccgcgcgtcaaggcgctgagccgcgtgagccgcggcaatgcgttgctcacagagttgctgggggcagtgcttgatgtctcggggccgctgcgcagaagcctgatcgcgcgctttgtcacgggcggggcaaacctggccagcctgctggcggatgagtccgcgctggagggcttcgtgcgtcagagcgtcttcggggtctggcatgccagcggcacttgccggatgggcgcgcatgcggaccggagcgcggtgacggatgcggcgggccgcgttcacgatgttggcaggctgcgcgtcgtagacgcctctctgatgccgcggctgccgacggccaataccaacatccccaccatcatgctcgcggaaaagattgccgacaccatgcaggccgagcgccgcgcagtccggccggcatcgagcgaagttgcccatccgggttgaagaccgcagcgcaaatccacgctgaagaacgaagctgcacgatggcgcgccaccggcggctggaacgaggagataacgatgaaatacgatgacgaaatccgcgcgcgctcttaccgcttccgcgacgaatatgtcgccgccacccctgcgtggtatcgcggcgaattgcatctggccttcacgctgctgttcaccggcggggtgattgcctggtgcgcgatgaaactgcaagcaccgacgctggcgcagtggctcgcgatcgtgccaatcttcttgctcgggaactgggccgagtgggccgcgcaccgctatatattgcatcgaccgacgcgcttgttcagcgcgatctataaacggcattgcgcggtgcatcatcgcttctttacacatctgacgcttgagtacaaaggccagaagcactggcgcgccttgttgtttccgccctttgcgccggtagcctttgtgctggcggccgtaccgttcgcgctggtgatcggcttggggttctcgaagaacgcgggctatatcgcgctgatgacgatggcggcgtactacctgatgtacgagggcctgcatacgctgtcgcacatcacggaaagcccactgctggaccggatgccgttcgtggggaccgtgcggcgcctgcacgtcacgcatcacgatcctgagctgatggccacgcagaacttcaacctgaccttcccgatctgcgacacgctgttcggcacgcgcagcgatgtgccacacgaggtgcgaagcccgatgcaagggcaggggtagccagggccgccgcacccggatcagccggcggcgcctgacaccctgcggatctcctcggccacgatcgcgaccagcgcaacggctgccggggcggcaaggcaccgcccgcgcgacaaccctgccgtcatgatccaagcagagattcaaatcaaatcaaaggagacaacaatggaagccgtagcaaaaaagcgtacagagacgatcggcgaggcgctgccagcggcgagcaatcgccaggtgtttggtgccgtgacggcgtcgtgcatgggatgggcgctggacctgttcgacctgttcatcctgctgttcgtggcgcccgtgatcggcaggctgtttttcccgtcggagcacgcgatgctgtcgctggcggcggtgtacgcgtcgtttgccgtgacgctgctgatgcggccactcggctcggcgatcttcggctcttatgccgaccgccacggccgcaagggggcgatggtggttgccgtcactggcgttggcttgtccacggcggcgttcggcctgctgccgacggtgagtcaggtggggctgcttgcgccagccttgtttatcctgctgcggctggtgcagggcatcttcgtgggtggcgtggtggcatccacccacaccatcggtaccgaatcggtgcccccgtcctggcgcggggccgtttccgggctggtcggtggcggtggcgcgggtatcggggcactgctggcttccattacctacatggcgatgaccgcgctgtttccgggggaagcgttcgacgcctggggttggcgctgcatgttcttctccggcatcatcagctcggtgctcggcctgttcatcttcaactcgctggaggagtctccgctgtggaagcagttgcaggcggccaaggggcacgccgcgccggttgagaacccgctgcgcgtgatcttctcccgccagtaccgtggcgtcctcttcgtcaacatcctgctcaccgtgggcggtggcagcgcctactacctgacctccggctatctgccgaccttcctcaagatcgtggtaaaggcaccggcgggggcctccgcggccatcctgatggccagcagcgttggcgttatcgtggcttcgatacttgccggtcacctcagcacgctgattggtcgcaagcgagccttcctgctgatcggcgccttgaacgtggtgctgctgccgttgatctaccaacggatgtccgcggtaccggatgtcaccacacttggcctgtatgccgtggcgctggcgatgctgggcagcaccggcttcgccccgatcctcattttcctgaacgaacggtttcccaccagcatccgtgccacggggaccggcctgtcatggaatatcggctttgccatcggcggcatgatgccgacctttgcgtcgctgtgcgccagcacccccgccgacctgccaaaagtgctggggatcttcgtcgcggtggtcactgccatttacctggccggtgcggcgatcgttcctgagaccgccggccgccttggggaggtcagccagcccgagcggtag

SEQ ID NO: 25 - DNA, 1494 bp, Cupriavidus basilensis
  source 1..1494: mol_type = genomic DNA
atgtccaggcaacccgccgaaaagacgaacagcgcgtcgcaagccgcgccattgcccgaagggccgctgaccctgcgcagctggctgcgccacctcggcaacacggaccggctggctgcaatcgacgagccggtggcactagagcacaccctcgcagccgtcgccaagcggctcgatggcgagcgcgcggcgttcttccgccggcctggcggccacgccgttccggtcgtgagcgggttcatgtcgcgccgcgcgtggatcgccgaggcgatgggcgtgcccgaggccggcttgctcgagcgcgtgcgcagcgccgccgcgcagcccttgccggtgagcgaggtggcccagggcgaggccgcatgccagcaggtcatccacctggacaaggtggacctgcgcaagctgttgcccatcccgacccacagcgagcatgacaacggcccctatatcactgcaggcctggccatcgcgcgcaacccgcgcaccggcgtgcagaacgtatcgatccaccgcatccaggtgcatgccgcggatcgcatggccatcctgatgctgccgcggcatctcgatgcgttctaccgtgcggcggaggaatgtggcgaggcgctgccgattgccattgtcatcggtgtcgatccgctcaccatgctggcttcgcaggccatcacgcctatcgaccatgacgagttggagatcgccggggcattgcacggtgcgccgctggaagtgatcaagtgccgcaccagcgatgtgcgtgtgccggccaatgccgagatcgtgatcgagggccggcttctgcccggcgagcgcgagatggaagggccctttggcgaatttcccaagtactacagcagcgccgagccgcgcgaggtcatccaggtcgacgccgtcacgcaccgtcatcgcccgatctatcacaccatcgtgccggcggagatggagcacctgctgctcggggcgattccgcgcgaggcgaccttgctggcgcatctgcagcgcagccatcccggggtgcaggatgtgcatctgtcggtgggcggcgtatgccggtaccacttgtacgtaaagctcgacaagaagcgcgagggcgaagcgaagaacgtcattctcgcggcgtttggcgcgcactacgacatcaagcaggtggtcgtggtggataccgatgtcgacgtccacgacccggccgaggtggaatgggccgtcgcgacccgcttccaggcagaccaggacctggtggtgatcgccggggcgcagggctcggtgctcgacccctccacgaccgtcgccgccaacctcgccggcatcgacaatcccgagccgcacctgcaaggcatctgcgccaagatggggctggacgcgacccgcccggtcaagtacgcggcgcatgtgttcacccgcgtgcggattcccggcgagtcaaccatcgatttgcaggcgctggtgtcggtcgacccatcgcactgggaatcgtatctcggcgaaggagcttga

SEQ ID NO: 26 - protein, 497 aa, Cupriavidus basilensis
MSRQPAEKTNSASQAAPLPEGPLTLRSWLRHLGNTDRLAAIDEPVALEHTLAAVAKRLDGERAAFFRRPGGHAVPVVSGFMSRRAWIAEAMGVPEAGLLERVRSAAAQPLPVSEVAQGEAACQQVIHLDKVDLRKLLPIPTHSEHDNGPYITAGLAIARNPRTGVQNVSIHRIQVHAADRMAILMLPRHLDAFYRAAEECGEALPIAIVIGVDPLTMLASQAITPIDHDELEIAGALHGAPLEVIKCRTSDVRVPANAEIVIEGRLLPGEREMEGPFGEFPKYYSSAEPREVIQVDAVTHRHRPIYHTIVPAEMEHLLLGAIPREATLLAHLQRSHPGVQDVHLSVGGVCRYHLYVKLDKKREGEAKNVILAAFGAHYDIKQVVVVDTDVDVHDPAEVEWAVATRFQADQDLVVIAGAQGSVLDPSTTVAANLAGIDNPEPHLQGICAKMGLDATRPVKYAAHVFTRVRIPGESTIDLQALVSVDPSHWESYLGEGA

SEQ ID NO: 27 - DNA, 594 bp, Cupriavidus basilensis
  source 1..594: mol_type = genomic DNA
atggtcagccagagacgcatcattgtcggtatatccggcgccagcggcgcggccatcggcgtcaatctcctcaaggcgatgcgcaacctggacggcgtcgagtcgcacctcatcgtgtcggcgtccggcatgctcaccgcgacccaggaactcggcatcaagcgcagcgaactcgaggcacttgccgacgtagtgcataacgtgcgcgatattggcgcagcggtggccagcggctctttcgtgaccgagggcatggtggtcgcgccgtgctcgatgaagacactagcgtcggtggccaacgggttttccgacaacctgctgacccgcgccgcggacgtggtgctcaaggagcggcggcgcctggtgctggtggcgcgcgaaaccccgctgaacctcgcgcacctgcgcaacatgctgcacgccaccgagatgggcgcgatcgtgatgccgcccgtgcccgccttctactcgcatccgaccagcatcgaggatgtggtcaatcacaccgtgggccgcatcctggacctgttccagatcgagcacgacacgctggtcagccgctggtcgggcctcgctcacgactttgccggctga

SEQ ID NO: 28 - protein, 197 aa, Cupriavidus basilensis
MVSQRRIIVGISGASGAAIGVNLLKAMRNLDGVESHLIVSASGMLTATQELGIKRSELEALADVVHNVRDIGAAVASGSFVTEGMVVAPCSMKTLASVANGFSDNLLTRAADVVLKERRRLVLVARETPLNLAHLRNMLHATEMGAIVMPPVPAFYSHPTSIEDVVNHTVGRILDLFQIEHDTLVSRWSGLAHDFAG

SEQ ID NO: 29 - DNA, 963 bp, Cupriavidus basilensis
  source 1..963: mol_type = genomic DNA
atgaaaacttggctcgcccatgccgggctcgctttgctgttgctcacggggcttgcccatgcgcaaggctatcccaccaagccgatccgcatcgtggtgccgtacccgcccggcggcttcaacgacacgctggcccgcatcgtcggcagcaggctcaccgcagcctggggccagcccgtggtggtggacaacaagcccggcgcgggcaccatcatcggcacctcgttcgtggccaaggccgcgcccgatggctacacgctgctggtggtgcagttccccttcggtgccaatccctggctctataagtcgctgccgtacgacaccctcaaggacttcacgccggtcatcctggcaggcgagtcgccgatgacccttgtggtgaccaacggatcgccgatacgctccgtggacgatcttgtcaaatccgccaagggcacgcccggcaagatcaactacggctcgtcgggcagcggctcgtccaaccaccttgccatggcgttgttcgagcgcagtgccggcatcacgctcgcccaggttccctacaagggcagcacgcccatgctgaccgatctggccggcgggcaggtcgaagtggccttcgacgcgttgccccatgtgttgcctttcgtgaggtccggcaaggtacgcgcacttgccgtcgccgaccggagccgctttgcgtcgcttgccacggtgcccaccatggccgagagcggcctgcccggctacgacgcgtcatcctggcacggcattgtcgcgccggccggcacgcccccggagattgtgcaaaagctgaacgcgcagatcaacgacgcgctgcgcacggcggatgtgcgcaagctcttccacgagcagggcgtgcgtcccgacggcggcagccctgccgacttctctgcgtttatcggcaaggaaatggcgaagtggaagcaggtggtacatgacgcggccatccccttgcaatga

SEQ ID NO: 30 - protein, 320 aa, Cupriavidus basilensis
MKTWLAHAGLALLLLTGLAHAQGYPTKPIRIVVPYPPGGFNDTLARIVGSRLTAAWGQPVVVDNKPGAGTIIGTSFVAKAAPDGYTLLVVQFPFGANPWLYKSLPYDTLKDFTPVILAGESPMTLVVTNGSPIRSVDDLVKSAKGTPGKINYGSSGSGSSNHLAMALFERSAGITLAQVPYKGSTPMLTDLAGGQVEVAFDALPHVLPFVRSGKVRALAVADRSRFASLATVPTMAESGLPGYDASSWHGIVAPAGTPPEIVQKLNAQINDALRTADVRKLFHEQGVRPDGGSPADFSAFIGKEMAKWKQVVHDAAIPLQ

SEQ ID NO: 31 - DNA, 1740 bp, Cupriavidus basilensis
  source 1..1740: mol_type = genomic DNA
atggatacgccgagggagcgtttcgactacgtgattgttggcggcgggtccgccggttgcgtactggccaatcgcctgtcgcaggacccggccatccgcgtcgcgctgatcgaggggggcatcgatacgccgccggacgctgtgccggcggagatcctcgacagctatccgatgcccttgttctacggtgaccggtatatctggccatcgctgcaagcccgcgccgtggcagggggcaggtccaaggtctacgagcaggggcgcgtcatgggcggcggctccagcatcaacgtgcaggccgcaaaccgcgggctgccgcgcgactacgatgagtgggccgcgttgggcgcgtccggctggtcgtggcaggatgtgctgccgtacttccgccgccttgagcgcgatgtggattacggcaacagcccgctgcacggcagccacggaccggtgccgatccgccgcatcctgccgcaggcttggccgccgttctgcacggagtttgcgcaagcgatgggccgcagcggcctgtccgagctggccgaccagaacgcggagttcggcgatggctggtttccggccgccttctcgaacctggatgacaaacgggtttcgaccgccatcgcctatctcgacgcggatacgcgccggcgggtcaatctgcggatctatgccgagacaacggtgcgcaagctcgtcgtatccggccgggaagcgcgtggggtgatcgccatccgggccgatgggtcgcggctggcgctggacgccggggaggtcatcgtgtccgcgggcgccttgcagtcgcccgccatcctgatgcgcgcggggatcggcgacgctggcgcgctgcaggccctcggcatcgaggtcgtagccgaccggcccggcgttggccgcaatctccaggatcatccggcgctgacgttctgccagttcctcgcgccccagtaccgcatgccgctctcgcgccggcgcgccagcatgacggcggcgcgcttctcatcggaggtgccaggcggcgaggcgtcggacatgtacctgtccagttccacgcgggcgggctggcatgcactcggtaatcggctcggcctcttcttcctgtggtgcaatcggccattctcgcgcgggcaggttcgccttgcgggagcccagccggatatgccgcccgtggtggagctcaacctgctcgacgacgagcgggatctgcggcgcatggtggccggcgtacgcaagttggtgcagatcgtgggtgcgtcggccttgcatcagcatccgggtgatttcttccccgctacgttttcgccgcgcgtcaaggcgctgagccgcgtgagccgcggcaatgcgttgctcacagagttgctgggggcagtgcttgatgtctcggggccgctgcgcagaagcctgatcgcgcgctttgtcacgggcggggcaaacctggccagcctgctggcggatgagtccgcgctggagggcttcgtgcgtcagagcgtcttcggggtctggcatgccagcggcacttgccggatgggcgcgcatgcggaccggagcgcggtgacggatgcggcgggccgcgttcacgatgttggcaggctgcgcgtcgtagacgcctctctgatgccgcggctgccgacggccaataccaacatccccaccatcatgctcgcggaaaagattgccgacaccatgcaggccgagcgccgcgcagtccggccggcatcgagcgaagttgcccatccgggttga

SEQ ID NO: 32 - protein, 579 aa, Cupriavidus basilensis
MDTPRERFDYVIVGGGSAGCVLANRLSQDPAIRVALIEGGIDTPPDAVPAEILDSYPMPLFYGDRYIWPSLQARAVAGGRSKVYEQGRVMGGGSSINVQAANRGLPRDYDEWAALGASGWSWQDVLPYFRRLERDVDYGNSPLHGSHGPVPIRRILPQAWPPFCTEFAQAMGRSGLSELADQNAEFGDGWFPAAFSNLDDKRVSTAIAYLDADTRRRVNLRIYAETTVRKLVVSGREARGVIAIRADGSRLALDAGEVIVSAGALQSPAILMRAGIGDAGALQALGIEVVADRPGVGRNLQDHPALTFCQFLAPQYRMPLSRRRASMTAARFSSEVPGGEASDMYLSSSTRAGWHALGNRLGLFFLWCNRPFSRGQVRLAGAQPDMPPVVELNLLDDERDLRRMVAGVRKLVQIVGASALHQHPGDFFPATFSPRVKALSRVSRGNALLTELLGAVLDVSGPLRRSLIARFVTGGANLASLLADESALEGFVRQSVFGVWHASGTCRMGAHADRSAVTDAAGRVHDVGRLRVVDASLMPRLPTANTNIPTIMLAEKIADTMQAERRAVRPASSEVAHPG

SEQ ID NO: 33 - DNA, 702 bp, Cupriavidus basilensis
  source 1..702: mol_type = genomic DNA
atgaaatacgatgacgaaatccgcgcgcgctcttaccgcttccgcgacgaatatgtcgccgccacccctgcgtggtatcgcggcgaattgcatctggccttcacgctgctgttcaccggcggggtgattgcctggtgcgcgatgaaactgcaagcaccgacgctggcgcagtggctcgcgatcgtgccaatcttcttgctcgggaactgggccgagtgggccgcgcaccgctatatattgcatcgaccgacgcgcttgttcagcgcgatctataaacggcattgcgcggtgcatcatcgcttctttacacatctgacgcttgagtacaaaggccagaagcactggcgcgccttgttgtttccgccctttgcgccggtagcctttgtgctggcggccgtaccgttcgcgctggtgatcggcttggggttctcgaagaacgcgggctatatcgcgctgatgacgatggcggcgtactacctgatgtacgagggcctgcatacgctgtcgcacatcacggaaagcccactgctggaccggatgccgttcgtggggaccgtgcggcgcctgcacgtcacgcatcacgatcctgagctgatggccacgcagaacttcaacctgaccttcccgatctgcgacacgctgttcggcacgcgcagcgatgtgccacacgaggtgcgaagcccgatgcaagggcaggggtag

SEQ ID NO: 34 - protein, 233 aa, Cupriavidus basilensis
MKYDDEIRARSYRFRDEYVAATPAWYRGELHLAFTLLFTGGVIAWCAMKLQAPTLAQWLAIVPIFLLGNWAEWAAHRYILHRPTRLFSAIYKRHCAVHHRFFTHLTLEYKGQKHWRALLFPPFAPVAFVLAAVPFALVIGLGFSKNAGYIALMTMAAYYLMYEGLHTLSHITESPLLDRMPFVGTVRRLHVTHHDPELMATQNFNLTFPICDTLFGTRSDVPHEVRSPMQGQG

SEQ ID NO: 35 - DNA, 1359 bp, Cupriavidus basilensis
  source 1..1359: mol_type = genomic DNA
atggaagccgtagcaaaaaagcgtacagagacgatcggcgaggcgctgccagcggcgagcaatcgccaggtgtttggtgccgtgacggcgtcgtgcatgggatgggcgctggacctgttcgacctgttcatcctgctgttcgtggcgcccgtgatcggcaggctgtttttcccgtcggagcacgcgatgctgtcgctggcggcggtgtacgcgtcgtttgccgtgacgctgctgatgcggccactcggctcggcgatcttcggctcttatgccgaccgccacggccgcaagggggcgatggtggttgccgtcactggcgttggcttgtccacggcggcgttcggcctgctgccgacggtgagtcaggtggggctgcttgcgccagccttgtttatcctgctgcggctggtgcagggcatcttcgtgggtggcgtggtggcatccacccacaccatcggtaccgaatcggtgcccccgtcctggcgcggggccgtttccgggctggtcggtggcggtggcgcgggtatcggggcactgctggcttccattacctacatggcgatgaccgcgctgtttccgggggaagcgttcgacgcctggggttggcgctgcatgttcttctccggcatcatcagctcggtgctcggcctgttcatcttcaactcgctggaggagtctccgctgtggaagcagttgcaggcggccaaggggcacgccgcgccggttgagaacccgctgcgcgtgatcttctcccgccagtaccgtggcgtcctcttcgtcaacatcctgctcaccgtgggcggtggcagcgcctactacctgacctccggctatctgccgaccttcctcaagatcgtggtaaaggcaccggcgggggcctccgcggccatcctgatggccagcagcgttggcgttatcgtggcttcgatacttgccggtcacctcagcacgctgattggtcgcaagcgagccttcctgctgatcggcgccttgaacgtggtgctgctgccgttgatctaccaacggatgtccgcggtaccggatgtcaccacacttggcctgtatgccgtggcgctggcgatgctgggcagcaccggcttcgccccgatcctcattttcctgaacgaacggtttcccaccagcatccgtgccacggggaccggcctgtcatggaatatcggctttgccatcggcggcatgatgccgacctttgcgtcgctgtgcgccagcacccccgccgacctgccaaaagtgctggggatcttcgtcgcggtggtcactgccatttacctggccggtgcggcgatcgttcctgagaccgccggccgccttggggaggtcagccagcccgagcggtag

SEQ ID NO: 36 - protein, 452 aa, Cupriavidus basilensis
MEAVAKKRTETIGEALPAASNRQVFGAVTASCMGWALDLFDLFILLFVAPVIGRLFFPSEHAMLSLAAVYASFAVTLLMRPLGSAIFGSYADRHGRKGAMVVAVTGVGLSTAAFGLLPTVSQVGLLAPALFILLRLVQGIFVGGVVASTHTIGTESVPPSWRGAVSGLVGGGGAGIGALLASITYMAMTALFPGEAFDAWGWRCMFFSGIISSVLGLFIFNSLEESPLWKQLQAAKGHAAPVENPLRVIFSRQYRGVLFVNILLTVGGGSAYYLTSGYLPTFLKIVVKAPAGASAAILMASSVGVIVASILAGHLSTLIGRKRAFLLIGALNVVLLPLIYQRMSAVPDVTTLGLYAVALAMLGSTGFAPILIFLNERFPTSIRATGTGLSWNIGFAIGGMMPTFASLCASTPADLPKVLGIFVAVVTAIYLAGAAIVPETAGRLGEVSQPER

SEQ ID NO: 37 - DNA, 29 bp, synthetic construct
  source 1..29: mol_type = other DNA
ccgctcgaggtgttccaggaccagcagtc

SEQ ID NO: 38 - DNA, 30 bp, synthetic construct
  source 1..30: mol_type = other DNA
acgcctgctgccaaccagcaggtgcttcat

SEQ ID NO: 39 - DNA, 31 bp, synthetic construct
  source 1..31: mol_type = other DNA
tgctggttggcagcaggcgttggatcactga

SEQ ID NO: 40 - DNA, 28 bp, synthetic construct
  source 1..28: mol_type = other DNA
cccaagcttcagtcgtcgaaggccagca

SEQ ID NO: 41 - DNA, 28 bp, synthetic construct
  source 1..28: mol_type = other DNA
cccaagcttgtagacgtgcagcacgctg

SEQ ID NO: 42 - DNA, 32 bp, synthetic construct
  source 1..32: mol_type = other DNA
ctcggctaaaggcagttggaacatgagatagc

SEQ ID NO: 43 - DNA, 29 bp, synthetic construct
  source 1..29: mol_type = other DNA
caactgcctttagccgagtaagcgacacc

SEQ ID NO: 44 - DNA, 29 bp, synthetic construct
  source 1..29: mol_type = other DNA
ccgctcgaggcagtgccgaggtgtcgaag

SEQ ID NO: 45 - DNA, 1344 bp, Pseudomonas putida
  source 1..1344: mol_type = genomic DNA
atggaacagcgcaaacgcatcaagacactgggatcgttggccttgcttgcacttgtaggcagcagcggtacacaggctgccgaggctttttccagcgaatccaaatggatgaccggcgactggggcggcacccggaccgagctgctggacaagggctatgacttcaccctcgattatgtgggtgaggtggctggcaacctgcatggcggctacaacgacgacaagacggcacgctacagcgaccagttcgccctcggcgcgcatctggacttgcagaagatactgggctggcatgatgccgagttcaagctggcaatcaccgagcgaagcggtcgcaacctgtccaacgaccgcatcagcgacccgcgcgccgggcagttcagctcggtgcaggaggtgtggggccgtggccagacctggcgcctgacccagatgtggatcaagcagaagtacttcgacggcgcgctggacgtgaaatttggccgttttggcgagggcgaggacttcaacagcttcccttgcgacttccagaacctggccttctgcggctcgcaggtgggcaactgggtgggcggcatctggtacaactggccggtcagccagtgggcgctgcgggtgaagtacaacatcacgccggagttcttcgtacaggtcggggccttcgagcagaacccttccaacctggaaaccggcaacggcttcaagctcagcggcagtggtaccaagggggcgatcttgccggtggaagcggtgtggtcgcccaaggtcaatggcctgccgggcgagtaccgcctgggttactactacagcacggccaaggctgacgatgtgtacgacgacgtcaacggcaacccgcaggcgctgacaggtgaagccttcaagtcgcactccagcaagcacggatggtgggtggtggcgcagcagcaggtcactgcccatggcggcgacgtcaaccggggcctcagcctgttcgccaacttcaccgtgcacgacaaggccaccaacgtggtcgacaactaccagcaggtggggctggtctacaaaggcgctttcgacgcccggcccaaggatgacatcggcttcggcgtggcgcgtattcatgtgaatgacgacgtgaagaagcgcgccgaactgctcaacgcacagagcggcatcaacgattacgacaaccctggtttcgtgccgctgcagcgtaccgaatacaacgcagagctctactacggcttccacgttaccaactggctgaccgtgaggcccaacctgcagtacatcaagagccctggcggggtggacgaggtggataacgcgctggtcgctggcttgaagattcagtcgtcattctga

SEQ ID NO: 46 - protein, 447 aa, Pseudomonas putida
MEQRKRIKTLGSLALLALVGSSGTQAAEAFSSESKWMTGDWGGTRTELLDKGYDFTLDYVGEVAGNLHGGYNDDKTARYSDQFALGAHLDLQKILGWHDAEFKLAITERSGRNLSNDRISDPRAGQFSSVQEVWGRGQTWRLTQMWIKQKYFDGALDVKFGRFGEGEDFNSFPCDFQNLAFCGSQVGNWVGGIWYNWPVSQWALRVKYNITPEFFVQVGAFEQNPSNLETGNGFKLSGSGTKGAILPVEAVWSPKVNGLPGEYRLGYYYSTAKADDVYDDVNGNPQALTGEAFKSHSSKHGWWVVAQQQVTAHGGDVNRGLSLFANFTVHDKATNVVDNYQQVGLVYKGAFDARPKDDIGFGVARIHVNDDVKKRAELLNAQSGINDYDNPGFVPLQRTEYNAELYYGFHVTNWLTVRPNLQYIKSPGGVDEVDNALVAGLKIQSSF

SEQ ID NO: 47 - DNA, 909 bp, Pseudomonas putida
  source 1..909: mol_type = genomic DNA
atgacaacgaccaccgcccaactgcgggcctcacccctggacgcgcttcagcgctggctgcccaagctggtgctggcacccagcatgttcatcgtcctggtgggcttctacggctacatcctctggaccttcgtgctgtccttcaccacctcgacctttctgcccacctacaagtgggcgggccttgcgcaatacgcccgtctgttcgacaacgaccgctggtgggtggcgagcaagaacctgcttctgttcggcggcctgttcatcgccatcagcctggccatcggtgtgttgctggcggtgctgctggaccagcgcatccgtcgcgagggcttcattcgcaccatttacctgtaccccatggcactgtcgatgatcgtcaccggcacggcctggaagtggctgctcaacccgggcatgggcctggacaagctgctgcgcgactggggctgggagggctttcgcctggactggctgatcgatcccgaccgggtggtgtattgcctggtgatcgcggccgtgtggcaggcttcgggcttcatcatggccatgttccttgccggcttgcgtggcgtcgacccgtcgatcatccgcgctgcgcagatggatggcgccagcctgccgcgcatctactggaccgtggtgctgcccagcctgcgcccggtgttcttcagcgcgctgatgatcctctcgcacattgccatcaagagcttcgacctggtggcggcaatgacggccggcggcccgggttactcctccgacttgccggccatgttcatgtactcgttcaccttcagccgcggccagatgggcatgggctcggccagcgccatcctgatgctcggggcaatcctggcgatcctcgtgccttacctgtactcggagctgcggagcaaacgccatgcatag

SEQ ID NO: 48 - protein, 302 aa, Pseudomonas putida
MTTTTAQLRASPLDALQRWLPKLVLAPSMFIVLVGFYGYILWTFVLSFTTSTFLPTYKWAGLAQYARLFDNDRWWVASKNLLLFGGLFIAISLAIGVLLAVLLDQRIRREGFIRTIYLYPMALSMIVTGTAWKWLLNPGMGLDKLLRDWGWEGFRLDWLIDPDRVVYCLVIAAVWQASGFIMAMFLAGLRGVDPSIIRAAQMDGASLPRIYWTVVLPSLRPVFFSALMILSHIAIKSFDLVAAMTAGGPGYSSDLPAMFMYSFTFSRGQMGMGSASAILMLGAILAILVPYLYSELRSKRHA

SEQ ID NO: 49 - DNA, 1188 bp, Pseudomonas putida
  source 1..1188: mol_type = genomic DNA
atgcacaacaacaataagcacctgccgcctcgttttctcgcagccgccatcgccagcttttctgcgcttggcctgagcagcgtcgccgaagccgagatcatgctgtacgacaaggaccagacgacgttttccaccgatggctatatcaacgccttctacgtcaacagcgaggtcgaccgtgagggcgagcagtttgaccgccgccagtcgcgggtgaagatgggcttcttgcccaactacctaggcttcaacatgggcaagcaggtggatgacctgaaactcggcgcgcgtgcctcgttctgggtaaccatcaacgacagtgaaaccaacggcaccgacaccgccatcgacgtgcgccagttctatggcacggtggccaaccccgagtggggcgaggtgctgatcggcaaggacttcgggctgttcgcccgatccaacatcctgctcgacgaactgctggccggttatggccaggtcagcgacaccctggggctggtggacggcggcggggtgtcgttcggcaacattggcagcggttacccatacccgttccctacctcacagatcacctaccgtacgcccgtgatggagggcctacgggttgcggtgggcatcatggacccggtggacaccaacgacagcagcccgaccggaaaggcctaccaggaaaacccacgcaccgagagcgagatcacctaccagttcgacctcggtggcgcgcagatctacagttgggtcaacggcagttaccagacctcggacaatactgactccacggtagaaacgatcacttccaagggggtgggttacggggtgcaggcaaagatgggcggctggtcgctgaccggctcggggttccaggccaaaggcatcaacccgttcttcaccaacaatgccggcgaaccggttttgcgcaatgtcgacagtgatggctacctgctgcagggctcgtacaagttcggcaagaaccgcgtggcgctgtcctatggcaagaccaaggacgatggcaacggtgcggttggcagcggcgcggactacgagacccggggtgtggcgctgttccatgacatcaacgacaacctgaaactggtggccgagtacaaccagttttccatcgacgggcatgacaccagtgcgcagaacgaagacaccgatacctttgcggtgggcgcggtgttgacctggtaa

SEQ ID NO: 50 - protein, 395 aa, Pseudomonas putida
MHNNNKHLPPRFLAAAIASFSALGLSSVAEAEIMLYDKDQTTFSTDGYINAFYVNSEVDREGEQFDRRQSRVKMGFLPNYLGFNMGKQVDDLKLGARASFWVTINDSETNGTDTAIDVRQFYGTVANPEWGEVLIGKDFGLFARSNILLDELLAGYGQVSDTLGLVDGGGVSFGNIGSGYPYPFPTSQITYRTPVMEGLRVAVGIMDPVDTNDSSPTGKAYQENPRTESEITYQFDLGGAQIYSWVNGSYQTSDNTDSTVETITSKGVGYGVQAKMGGWSLTGSGFQAKGINPFFTNNAGEPVLRNVDSDGYLLQGSYKFGKNRVALSYGKTKDDGNGAVGSGADYETRGVALFHDINDNLKLVAEYNQFSIDGHDTSAQNEDTDTFAVGAVLTW

(19) United States
(12) Patent Application Publication
(10) Pub. No.: US 2024/0401067 A1
(43) Pub. Date: Dec. 5, 2024

(54) MICROBES AND METHODS FOR SELECTIVE DETOXIFICATION OF LIGNOCELLULOSIC BIOMASS

(71) Applicant: Wisconsin Alumni Research Foundation, Madison, WI (US)

(72) Inventor: Victor Ujor, Sun Prairie, WI (US)

(73) Assignee: Wisconsin Alumni Research Foundation, Madison, WI (US)

(21) Appl. No.: 18/672,924

(22) Filed: May 23, 2024

Related U.S. Application Data
(60) Provisional application No. 63/505,197, filed on May 31, 2023.

(51) Int. Cl. C12N 15/78; C07K 14/195; C12N 9/00; C12N 9/02; C12N 9/04; C12N 9/10; C12N 9/88

(52) U.S. Cl. CPC C12N 15/78; C07K 14/195; C12N 9/0006; C12N 9/001; C12N 9/1085; C12N 9/88; C12N 9/93; C12Y 103/99008; C12Y 602/01031

(57) ABSTRACT

Microbes and methods for selectively detoxifying lignocellulosic biomass, such as microbes and methods for removing furanic and phenolic aldehydes from lignocellulosic hydrolysates.

### STATEMENT REGARDING FEDERALLY SPONSORED RESEARCH

This invention was made with government support under 25-CRHF-0-6055 awarded by the USDA/NIFA. The government has certain rights in the invention.

### SEQUENCE LISTING

The instant application contains a Sequence Listing which has been submitted in XML format and is hereby incorporated by reference in its entirety. The XML copy, created on May 8, 2024, is named USPTO-240523-09824519-P230311US02-APP-SEQ_LIST and is 95,019 bytes in size.

### FIELD OF THE INVENTION

The invention is directed to microbes and methods for selectively detoxifying lignocellulosic biomass, such as microbes and methods for removing furanic and phenolic aldehydes from lignocellulosic hydrolysates.

### BACKGROUND

The use of lignocellulosic biomass as a renewable feedstock in biomanufacturing remains limited by several issues. A primary concern is that the commonly used acid- or alkali-based deconstruction strategies are efficient at releasing sugars (e.g., glucose and xylose), but also generate several co-products. These co-products include furanic and phenolic aledehydes, which are generally toxic to commonly used fermenting microorganisms. As a result, there continues to be a need for economical methods for removing these unwanted compounds from lignocellulosic biomass hydrolysates.

### SUMMARY OF THE INVENTION

One aspect of the invention is directed to recombinant microorganisms. In some versions, The recombinant microorganisms comprise one or more modifications with respect to a corresponding microorganism not comprising the one or more modifications.

In some versions, the one or more modifications comprise 1 or more, 2 or more, 3 or more, 4 or more, 5 or more, 6 or more, 7 or more, 8 or more, 9 or more, 10 or more, 11 or more, 12 or more, 13 or more, 14 or more, or each of: a functional deletion of a glucokinase gene present in the corresponding microorganism; a functional deletion of a quinoprotein glucose dehydrogenase gene present in the corresponding microorganism; a functional deletion of a carbohydrate transporter gene present in the corresponding microorganism; a recombinant gene encoding HmfA of Cupriavidus basilensis or a homolog thereof; a recombinant gene encoding HmfB of Cupriavidus basilensis or a homolog thereof; a recombinant gene encoding HmfC of Cupriavidus basilensis or a homolog thereof; a recombinant gene encoding HmfD of Cupriavidus basilensis or a homolog thereof; a recombinant gene encoding HmfE of Cupriavidus basilensis or a homolog thereof; a recombinant gene encoding HmfT1 of Cupriavidus basilensis or a homolog thereof; a recombinant gene encoding HmfF of Cupriavidus basilensis or a homolog thereof; a recombinant gene encoding HmfG of Cupriavidus basilensis or a homolog thereof; a recombinant gene encoding HmfH′ of Cupriavidus basilensis or a homolog thereof; a recombinant gene encoding HmfH of Cupriavidus basilensis or a homolog thereof; a recombinant gene encoding HmfS of Cupriavidus basilensis or a homolog thereof; and a recombinant gene encoding HmfT2 of Cupriavidus basilensis or a homolog thereof.

In some versions, the one or more modifications comprise one or more, two or more, or each of: a functional deletion of a glucokinase gene present in the corresponding microorganism; a functional deletion of a quinoprotein glucose dehydrogenase gene present in the corresponding microorganism; and a functional deletion of a carbohydrate transporter gene present in the corresponding microorganism.

In some versions, the one or more modifications comprise one or both of: a functional deletion of a glucokinase gene present in the corresponding microorganism; and a functional deletion of a quinoprotein glucose dehydrogenase gene present in the corresponding microorganism.

In some versions, the glucokinase gene is glk of Pseudomonas putida, glk of Escherichia coli, glk of Enterobacter hormaechei, or a homolog of any of the foregoing.

In some versions, the quinoprotein glucose dehydrogenase gene is gcd of Pseudomonas putida, gcd of Escherichia coli, gcd of Enterobacter hormaechei, or a homolog of any of the foregoing.

In some versions, the carbohydrate transporter gene is oprB-II of Pseudomonas putida or a homolog thereof.

In some versions, the glucokinase gene encodes a protein comprising an amino acid sequence with at least 80%, at least 85%, at least 90%, at least 95%, or at least 99% sequence identity to a sequence selected from the group consisting of SEQ ID NOS:2 and 8.

In some versions, the quinoprotein glucose dehydrogenase gene encodes a protein comprising an amino acid sequence with at least 80%, at least 85%, at least 90%, at least 95%, or at least 99% sequence identity to a sequence selected from the group consisting of SEQ ID NOS:4 and 10.

In some versions, the carbohydrate transporter gene encodes a protein comprising an amino acid sequence with at least 80%, at least 85%, at least 90%, at least 95%, or at least 99% sequence identity to a sequence selected from the group consisting of SEQ ID NOS:6, 46, 48, and 50.

In some versions, the wherein the one or more modifications comprise a functional deletion of a carbohydrate transporter gene present in the corresponding microorganism, wherein the carbohydrate transporter gene encodes a protein comprising an amino acid sequence with at least 80%, at least 85%, at least 90%, at least 95%, or at least 99% sequence identity to SEQ ID NO:6.

In some versions, the recombinant microorganism further comprises a functional deletion of one, two or three additional carbohydrate transporter genes present in the corresponding microorganism, wherein the additional carbohydrate transporter genes each encode a protein comprising an amino acid sequence with at least 80%, at least 85%, at least 90%, at least 95%, or at least 99% sequence identity to a sequence selected from the group consisting of SEQ ID NOS:46, 48, and 50.

In some versions, the recombinant microorganism further comprises a functional deletion of three additional carbohydrate transporter genes present in the corresponding microorganism, wherein the additional carbohydrate transporter genes encode: a protein comprising an amino acid sequence with at least 80%, at least 85%, at least 90%, at least 95%, or at least 99% sequence identity to SEQ ID NO:46; a protein comprising an amino acid sequence with at least 80%, at least 85%, at least 90%, at least 95%, or at least 99% sequence identity to SEQ ID NO: 48; a protein comprising an amino acid sequence with at least 80%, at least 85%, at least 90%, at least 95%, or at least 99% sequence identity to SEQ ID NO:50.

In some versions, the one or more modifications comprise one or more, two or more three or more, four or more, five or more, or each of: a recombinant gene encoding HmfA of Cupriavidus basilensis or a homolog thereof; a recombinant gene encoding HmfB of Cupriavidus basilensis or a homolog thereof; a recombinant gene encoding HmfC of Cupriavidus basilensis or a homolog thereof; a recombinant gene encoding HmfD of Cupriavidus basilensis or a homolog thereof; a recombinant gene encoding HmfE of Cupriavidus basilensis or a homolog thereof; and a recombinant gene encoding HmfT1 of Cupriavidus basilensis or a homolog thereof.

In some versions, the one or more modifications comprise one or more, two or more three or more, four or more, five or more, or each of: a recombinant gene encoding HmfF of Cupriavidus basilensis or a homolog thereof; a recombinant gene encoding HmfG of Cupriavidus basilensis or a homolog thereof; a recombinant gene encoding HmfH′ of Cupriavidus basilensis or a homolog thereof; a recombinant gene encoding HmfH of Cupriavidus basilensis or a homolog thereof; a recombinant gene encoding HmfS of Cupriavidus basilensis or a homolog thereof; and a recombinant gene encoding HmfT2 of Cupriavidus basilensis or a homolog thereof.

In some versions, the HmfA of Cupriavidus basilensis or the homolog thereof comprises an amino acid sequence with at least 80%, at least 85%, at least 90%, at least 95%, or at least 99% sequence identity to SEQ ID NO:13.

In some versions, the HmfB of Cupriavidus basilensis or the homolog thereof comprises an amino acid sequence with at least 80%, at least 85%, at least 90%, at least 95%, or at least 99% sequence identity to SEQ ID NO:15.

In some versions, the HmfC of Cupriavidus basilensis or the homolog thereof comprises an amino acid sequence with at least 80%, at least 85%, at least 90%, at least 95%, or at least 99% sequence identity to SEQ ID NO:17.

In some versions, the HmfD of Cupriavidus basilensis or the homolog thereof comprises an amino acid sequence with at least 80%, at least 85%, at least 90%, at least 95%, or at least 99% sequence identity to SEQ ID NO:19.

In some versions, the HmfE of Cupriavidus basilensis or the homolog thereof comprises an amino acid sequence with at least 80%, at least 85%, at least 90%, at least 95%, or at least 99% sequence identity to SEQ ID NO:21.

In some versions, the HmfT1 of Cupriavidus basilensis or the homolog thereof comprises an amino acid sequence with at least 80%, at least 85%, at least 90%, at least 95%, or at least 99% sequence identity to SEQ ID NO:23.

In some versions, the HmfF of Cupriavidus basilensis or the homolog thereof comprises an amino acid sequence with at least 80%, at least 85%, at least 90%, at least 95%, or at least 99% sequence identity to SEQ ID NO:26.

In some versions, the HmfG of Cupriavidus basilensis or the homolog thereof comprises an amino acid sequence with at least 80%, at least 85%, at least 90%, at least 95%, or at least 99% sequence identity to SEQ ID NO:28.

In some versions, the HmfH′ of Cupriavidus basilensis or the homolog thereof comprises an amino acid sequence with at least 80%, at least 85%, at least 90%, at least 95%, or at least 99% sequence identity to SEQ ID NO:30.

In some versions, the HmfH of Cupriavidus basilensis or the homolog thereof comprises an amino acid sequence with at least 80%, at least 85%, at least 90%, at least 95%, or at least 99% sequence identity to SEQ ID NO:32.

In some versions, the HmfS of Cupriavidus basilensis or the homolog thereof comprises an amino acid sequence with at least 80%, at least 85%, at least 90%, at least 95%, or at least 99% sequence identity to SEQ ID NO:34.

In some versions, the HmfT2 of Cupriavidus basilensis or the homolog thereof comprises an amino acid sequence with at least 80%, at least 85%, at least 90%, at least 95%, or at least 99% sequence identity to SEQ ID NO:36.

In some versions, the recombinant microorganism is an aerobic microorganism.

In some versions, the recombinant microorganism is a bacterium.

In some versions, the recombinant microorganism is an aerobic bacterium.

In some versions, the recombinant microorganism is from a genus selected from the group consisting of Pseudomonas, Escherichia, and Enterobacter.

In some versions, the recombinant microorganism is selected from the group consisting of Pseudomonas putida, Escherichia coli, and Enterobacter hormaechei.

In some versions, the recombinant microorganism is from a genus of Pseudomonas.

In some versions, the recombinant microorganism is Pseudomonas putida.

In some versions, the recombinant microorganism exhibits reduced consumption of a carbohydrate with respect to the corresponding microorganism.

In some versions, the recombinant microorganism exhibits increased consumption of a substituted furan with respect to the corresponding microorganism.

In some versions, the recombinant microorganism exhibits increased consumption of a substituted furan selected from the group consisting of furfural and hydroxymethylfurfural with respect to the corresponding microorganism.

Another aspect of the invention is directed methods of decreasing an amount of a substituted furan in a medium. In some versions, the methods comprise contacting the medium with a recombinant microorganism of the invention for a time sufficient to decrease the substituted furan in the medium.

In some versions, the substituted furan is selected from the group consisting of furfural and hydroxymethylfurfural.

In some versions, the medium comprises lignocellulosic biomass.

In some versions, the medium comprises lignocellulosic biomass hydrolysate.

In some versions, the contacting is performed under aerobic conditions.

The objects and advantages of the invention will appear more fully from the following detailed description of the preferred embodiment of the invention made in conjunction with the accompanying drawings.

### BRIEF DESCRIPTION OF THE DRAWINGS

FIG. 1. Glucose import machinery of Pseudomonas putida. Genes encoding OprB, Gcd and Glk (in light blue) are inactivated by homologous recombination.

FIG. 2. Schematic of homologous recombination-based gene deletion in P. putida.

FIGS. 3A and 3B. The growth profiles of P. putida knockout mutants on glucose relative to the wildtype. FIG. 3A. Optical densities. FIG. 3B. residual glucose concentrations.

FIGS. 4A and 4B. Strategy for enhancing inhibitor utilization in P. putida. FIG. 4A. Schematic of homologous recombination-based gene insertion into P. putida, showing the genetic map of furfural/HMF utilizing gene clusters of C. basilensis and a schematic of Gibson assembly used for joining DNA fragments. FIG. 4B. Agarose (1.2%) gel showing DNA fragments (linearized plasmid, PCR amplified hmf genes from genomic DNA of C. basilensis, and repairs arms amplified from genomic DNA of P. putida).

FIGS. 5A-5D. Growth kinetics of cultures of P. putida_control and P. putida_AT-FT supplemented with 10, 20, 30, and 40 mM furfural.

FIGS. 6A-6D. Furfural concentration profiles of cultures of P. putida_control and P. putida_AT-FT supplemented with 10, 20, 30, and 40 mM furfural.

FIGS. 7A-7D. Growth kinetics of cultures of P. putida_control and P. putida_AT-FT supplemented with 10, 20, 30, and 40 mM HMF.

FIGS. 8A-8D. HMF concentration profiles of cultures of P. putida_control and P. putida_AT-FT supplemented with 10, 20, 30, and 40 mM HMF.

FIG. 9. The growth profile of C. beijerinckii grown in glucose (60 g/L) and furfural (40 mM)-supplemented medium in which P. putida_AT-FT and P. putida_control were pre-grown.

### DETAILED DESCRIPTION OF THE INVENTION

One aspect of the invention is directed to recombinant microorganisms. The recombinant microorganisms of the invention comprise one or more modifications with respect to a corresponding microorganism not comprising the one or more modifications. The one or more modifications may confer reduced consumption of a sugar such as glucose with respect to the corresponding microorganism, increased consumption of a substituted furan such as furfural and hydroxymethylfurfural with respect to the corresponding microorganism, or any combination thereof.

“Corresponding microorganism” refers to a microorganism of the same species having the same or substantially same genetic and proteomic composition as a recombinant microorganism of the invention, with the exception of genetic and proteomic differences resulting from the modifications described herein for the recombinant microorganisms of the invention. In some versions, the corresponding microorganism is the native version of the recombinant microorganism of the invention, i.e., the unmodified microorganism as found in nature. The terms “microorganism” and “microbe” are used interchangeably herein.

The recombinant and/or corresponding microorganisms of the invention may comprise any type of microorganism. The recombinant and/or corresponding may be prokaryotic or eukaryotic. Suitable prokaryotes include bacteria and archaea. Suitable types of bacteria include α- and γ-proteobacteria, gram-positive bacteria, gram-negative bacteria, ungrouped bacteria, phototrophs, lithotrophs, and organotrophs. Suitable eukaryotes include yeast and other fungi. The recombinant and/or corresponding microorganism in some versions can be from an order selected from the group consisting of Pseudomonadales, Enterobacterales, and Sphingomonadales. The recombinant and/or corresponding microorganism in some versions can be from a family selected from the group consisting of Pseudomonadaceae, Enterobacteriaceae, and Sphingomonadaceae. The recombinant and/or corresponding microorganism in some versions can be from a genus selected from the group consisting of Pseudomonas, Escherichia, Enterobacter, Erythrobacter, Altererythrobacter Sphingomonas, Sphingobium, Sphingosinicella, Sphingopyxis, and Novosphingobium. An exemplary microorganism from the genus Pseudomonas is Pseudomonas putida. An exemplary microorganism from the genus Escherichia is Escherichia coli. An exemplary microorganism from the genus Enterobacter is Enterobacter hormaechei. Other examples of suitable microorganisms include Gram-positive bacteria such as strains of Bacillus, (e.g., B. brevis or B. subtilis), Lactobacillus, Lactococcus, or Streptomyces, or Gram-negative bacteria, such as strains of Salmonella, Vibrio, Corynebacterium, Ralstonia, Aeromonas or cyanobacteria, or oleaginous bacteria, such as Rhodococcus opacus, or Acinetobactor baylyi. Examples of suitable yeast cells include strains of Saccharomyces, such as S. cerevisiae or Lipomyces starkeyi; Schizosaccharomyces; Kluyveromyces; Pichia, such as P. pastoris or P. methlanolica, or P. stipitis; Hansenula, such as H. Polymorpha; Yarrowia; Candida; Cryptococcus; Basidiomycete, such as Rhodosporidium. Examples of suitable microalgal species Chlorophyta, such as chlorella; Bacillariophyceae, such as chaetoceros. Examples of suitable filamentous fungal cells include strains of Aspergillus, e.g., A. oryzae, A. niger, or A. nidulans; Fusarium or Trichoderma.

In some versions of the invention, the recombinant and/or corresponding microorganism is a microorganism incapable or minimally capable of consuming a sugar selected from the group consisting of glucose and xylose. In some versions of the invention, the recombinant and/or corresponding microorganism is a microorganism incapable or minimally capable of consuming glucose. In some versions of the invention, the recombinant and/or corresponding microorganism is a microorganism incapable or minimally capable of consuming xylose.

In some versions of the invention, the recombinant and/or corresponding microorganism is an aerobe or a facultative anaerobe. In some versions of the invention, the recombinant and/or corresponding microorganism is an aerobe. Aerobes are microorganisms that are able to live and reproduce only in the presence of free oxygen. Facultative anaerobes are microorganisms that are able to live and grow either with or without free oxygen.

The modifications of the invention may include a functional deletion of one or more genes. “Functional deletion” or its grammatical equivalents refers to any modification to a microorganism that ablates, reduces, inhibits, or otherwise disrupts production of a gene product, renders the gene product non-functional, or otherwise reduces or ablates the gene product's activity. “Gene product” refers to a protein or polypeptide encoded and produced by a particular gene. In some versions of the invention, functionally deleting a gene product or homolog thereof means that the gene is mutated to an extent that corresponding gene product is not produced at all.

One of ordinary skill in the art will appreciate that there are many well-known ways to functionally delete a gene product. For example, functional deletion can be accomplished by introducing one or more genetic modifications. As used herein, “genetic modifications” refer to any differences in the nucleic acid composition of a cell, whether in the cell's native chromosome or in endogenous or exogenous non-chromosomal plasmids harbored within the cell. Examples of genetic modifications that may result in a functionally deleted gene product include but are not limited to mutations, partial or complete deletions, insertions, and/or other variations to a coding sequence or a sequence controlling the transcription or translation of a coding sequence; placing a coding sequence under the control of a less active promoter; and expressing ribozymes or antisense sequences that target the mRNA of the gene of interest, etc. In some versions, a gene or coding sequence can be replaced with a selection marker or screenable marker. The genetic modifications that functionally delete a product of a particular gene can be cis-acting modifications (direct modifications of the particular gene itself) or trans-actin modifications (modifications other than to the particular gene itself that indirectly affect the gene). Various methods for introducing the genetic modifications described above are well known in the art and include homologous recombination, among other mechanisms. See, e.g., Green et al., Molecular Cloning: A laboratory manual, 4^{th} ed., Cold Spring Harbor Laboratory Press (2012) and Sambrook et al., Molecular Cloning: A Laboratory Manual, 3^{rd} ed., Cold Spring Harbor Laboratory Press (2001). Various other genetic modifications that functionally delete a gene product are described in the examples below.

In certain versions of the invention, the functionally deleted gene product may have less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1%, or about 0% of the activity of the non-functionally deleted gene product.

In certain versions of the invention, a microorganism with a functionally deleted gene product may have less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1%, or about 0% of the activity of the gene product compared to a microorganism with the non-functionally deleted gene product.

In certain versions of the invention, the functionally deleted gene product may be expressed at an amount less than about 95%, less than about 90%, less than about 85%, less than about 80%, less than about 75%, less than about 70%, less than about 65%, less than about 60%, less than about 55%, less than about 50%, less than about 45%, less than about 40%, less than about 35%, less than about 30%, less than about 25%, less than about 20%, less than about 15%, less than about 10%, less than about 5%, less than about 1%, or about 0% of the amount of the non-functionally deleted gene product.

In certain versions of the invention, the functionally deleted gene product may result from a genetic modification in which at least 1, at least 2, at least 3, at least 4, at least 5, at least 10, at least 20, at least 30, at least 40, at least 50, or more nonsynonymous substitutions are present in the gene or coding sequence of the gene product.

In certain versions of the invention, the functionally deleted gene product may result from a genetic modification in which at least 1, at least 2, at least 3, at least 4, at least 5, at least 10, at least 20, at least 30, at least 40, at least 50, or more bases are inserted in the gene or coding sequence of the gene product.

In certain versions of the invention, the functionally deleted gene product may result from a genetic modification in which at least about 1%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, or about 100% of the gene product's gene or coding sequence is deleted or mutated.

In certain versions of the invention, the functionally deleted gene product may result from a genetic modification in which at least about 1%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, or about 100% of a promoter driving expression of the gene product is deleted or mutated.

In certain versions of the invention, the functionally deleted gene product may result from a genetic modification in which at least about 1%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, or about 100% of an enhancer controlling transcription of the gene product's gene is deleted or mutated.

In certain versions of the invention, the functionally deleted gene product may result from a genetic modification in which at least about 1%, at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, at least about 90%, at least about 95%, or about 100% of a sequence controlling translation of gene product's mRNA is deleted or mutated.

In certain versions of the invention, the decreased activity or expression of the functionally deleted gene product is determined with respect to the activity or expression of the gene product in its unaltered state as found in nature. In certain versions of the invention, the decreased activity or expression of the functionally deleted gene product is determined with respect to the activity or expression of the gene product in its form in a corresponding microorganism. In certain versions, the genetic modifications giving rise to a functionally deleted gene product are determined with respect to the gene in its unaltered state as found in nature. In certain versions, the genetic modifications giving rise to a functionally deleted gene product are determined with respect to the gene in its form in a corresponding microorganism.

In some versions of the invention, a glucokinase gene is functionally deleted. Glucokinases have activity falling under Enzyme Commission (EC) EC 2.7.1.2. In some versions, the glucokinase gene is glk of Pseudomonas putida, glk of Escherichia coli, glk of Enterobacter hormaechei, or a homolog of any of the foregoing. In some versions, the glucokinase gene encodes a protein comprising an amino acid sequence with at least 80%, at least 85%, at least 90%, at least 95%, at least 99%, or 100% sequence identity to a sequence selected from the group consisting of SEQ ID NOS:2 and 8.

In some versions of the invention, a quinoprotein glucose dehydrogenase gene is functionally deleted. Quinoprotein glucose dehydrogenases have activity falling under EC 1.2.5.2. In some versions, the quinoprotein glucose dehydrogenase gene is gcd of Pseudomonas putida, gcd of Escherichia coli, gcd of Enterobacter hormaechei, or a homolog of any of the foregoing. In some versions, the quinoprotein glucose dehydrogenase gene encodes a protein comprising an amino acid sequence with at least 80%, at least 85%, at least 90%, at least 95%, at least 99%, or 100% sequence identity to a sequence selected from the group consisting of SEQ ID NOS:4 and 10.

In some versions of the invention, a carbohydrate transporter gene is functionally deleted. Carbohydrate transporter genes are genes encoding a protein that facilities transfer of a carbohydrate, such as glucose and/or xylose, into the cell. In some versions, the carbohydrate transporter gene is a glucose transporter gene. A glucose transporter gene is a gene encoding a protein that facilitates transfer of glucose into the cell. In some versions, the carbohydrate transporter gene is a xylose transporter gene. A xylose transporter gene is a gene encoding a protein that facilitates transfer of xylose into the cell. Examples of carbohydrate transporter genes include ATP-dependent cassette (ABC) transporter genes, sodium solute symporter genes, enzyme II integral membrane subunit genes of the bacterial PEP-dependent phosphotransferase system (PTS), and porin genes, such as carbohydrate-selective porins. In some versions, the carbohydrate transporter gene is a carbohydrate-selective porin gene. In some versions, the carbohydrate transporter gene is a carbohydrate-selective porin gene that facilitates transfer of glucose into the cell. In some versions, the carbohydrate transporter gene is oprB-II of Pseudomonas putida or a homolog thereof. In some versions, the carbohydrate transporter gene encodes a protein comprising an amino acid sequence with at least 80%, at least 85%, at least 90%, at least 95%, at least 99%, or 100% sequence identity to SEQ ID NO:6. In some versions, the carbohydrate transporter gene is oprB-1 of Pseudomonas putida or a homolog thereof. In some versions, the carbohydrate transporter gene encodes a protein comprising an amino acid sequence with at least 80%, at least 85%, at least 90%, at least 95%, at least 99%, or 100% sequence identity to SEQ ID NO:46. In some versions, the carbohydrate transporter gene is gtsB of Pseudomonas putida or a homolog thereof. In some versions, the carbohydrate transporter gene encodes a protein comprising an amino acid sequence with at least 80%, at least 85%, at least 90%, at least 95%, at least 99%, or 100% sequence identity to SEQ ID NO:48. In some versions, the carbohydrate transporter gene is KBDANE_14125 or PP_RS13865 of Pseudomonas putida or a homolog thereof. In some versions, the carbohydrate transporter gene encodes a protein comprising an amino acid sequence with at least 80%, at least 85%, at least 90%, at least 95%, at least 99%, or 100% sequence identity to SEQ ID NO:50.

In some versions of the invention, the one or more modifications comprise a genetic modification to include a recombinant gene. In most cases, the recombinant gene is configured to be expressed or overexpressed in the microorganism. If a cell endogenously comprises a particular gene, the gene may be modified to exchange or optimize promoters, exchange or optimize enhancers, or exchange or optimize any other genetic element to result in increased expression of the gene. Alternatively, one or more additional copies of the gene or coding sequence thereof may be introduced to the cell for enhanced expression of the gene product. If a microorganism does not endogenously comprise a particular gene, the gene or coding sequence thereof may be introduced to the microorganism for heterologous expression of the gene product. The gene or coding sequence may be incorporated into the genome of the microorganism or may be contained on an extra-chromosomal plasmid. The gene or coding sequence may be introduced to the microorganism individually or may be included on an operon. Techniques for genetic manipulation are described in further detail below.

In some versions of the invention, the one or more modifications comprise a recombinant gene encoding HmfA of Cupriavidus basilensis or a homolog thereof. The C. basilensis HmfA is the large subunit of a 2-furoyl-CoA dehydrogenase (EC 1.3.99.8). 2-Furoyl-CoA dehydrogenases catalyze the reaction: 2-Furoyl-CoA+Acceptor+H₂O<=>S-(5-Hydroxy-2-furoyl)-CoA+Reduced acceptor. The amino acid sequence of the C. basilensis HmfA is SEQ ID NO:13, which is encoded by SEQ ID NO:12. Homologs of the C. basilensis HmfA can include variants of the C. basilensis HmfA comprising an amino acid sequence with at least 80%, 85%, 90%, 95%, 99%, or 100% sequence identity to SEQ ID NO:13. Homologs of the C. basilensis HmfA can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfA are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfB of C. basilensis or a homolog thereof. The C. basilensis HmfB is the FAD-binding subunit of a 2-furoyl-CoA dehydrogenase (EC 1.3.99.8). 2-Furoyl-CoA dehydrogenases catalyze the reaction: 2-Furoyl-CoA+Acceptor+H₂O<=>S-(5-Hydroxy-2-furoyl)-CoA+Reduced acceptor. The amino acid sequence of the C. basilensis HmfB is SEQ ID NO:15, which is encoded by SEQ ID NO:14. Homologs of the C. basilensis HmfB can include variants of the C. basilensis HmfB comprising an amino acid sequence with at least 80%, 85%, 90%, 95%, 99%, or 100% sequence identity to SEQ ID NO:15. Homologs of the C. basilensis HmfB can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfB are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfC of C. basilensis or a homolog thereof. The C. basilensis HmfC is the 2Fe-2S iron sulfur subunit of a 2-furoyl-CoA dehydrogenase (EC 1.3.99.8). 2-Furoyl-CoA dehydrogenases catalyze the reaction: 2-Furoyl-CoA+Acceptor+H₂O<=>S-(5-Hydroxy-2-furoyl)-CoA+Reduced acceptor. The amino acid sequence of the C. basilensis HmfC is SEQ ID NO:17, which is encoded by SEQ ID NO:16. Homologs of the C. basilensis HmfC can include variants of the C. basilensis HmfC comprising an amino acid sequence with at least 80%, 85%, 90%, 95%, 99%, or 100% sequence identity to SEQ ID NO:17. Homologs of the C. basilensis HmfC can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfC are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169). In some versions of the invention, the recombinant microorganism comprises one or more genes encoding each of HmfA, HmfB, or HmfC, or homologs thereof.

In some versions of the invention, the one or more modifications comprise a recombinant gene encoding HmfD of C. basilensis or a homolog thereof. The C. basilensis HmfD is a furoyl-CoA synthetase (EC 6.2.1.31). Furoyl-CoA synthetases catalyze the reaction: ATP+2-Furoate+CoA<=>AMP+Diphosphate+2-Furoyl-CoA. The amino acid sequence of the C. basilensis HmfD is SEQ ID NO:19, which is encoded by SEQ ID NO:18. Homologs of the C. basilensis HmfD can include variants of the C. basilensis HmfD comprising an amino acid sequence with at least 80%, 85%, 90%, 95%, 99%, or 100% sequence identity to SEQ ID NO:19. Homologs of the C. basilensis HmfD can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfD are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfE of C. basilensis or a homolog thereof. The C. basilensis HmfE is a 2-oxoglutaroyl-CoA hydrolase. 2-Oxoglutaroyl-CoA hydrolases catalyze the reaction: 2-Oxoglutaryl-CoA+H2O<=>2-Oxoglutarate+CoA. The amino acid sequence of the C. basilensis HmfE is SEQ ID NO:21, which is encoded by SEQ ID NO:20. Homologs of the C. basilensis HmfE can include variants of the C. basilensis HmfE comprising an amino acid sequence with at least 80%, 85%, 90%, 95%, 99%, or 100% sequence identity to SEQ ID NO:21. Homologs of the C. basilensis HmfE can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfE are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfT1 of C. basilensis or a homolog thereof. The C. basilensis HmfT1 is a transporter protein in the Major Facilitator Superfamily. The amino acid sequence of the C. basilensis HmfT1 is SEQ ID NO:23, which is encoded by SEQ ID NO:22. Homologs of the C. basilensis HmfT1 can include variants of the C. basilensis HmfT1 comprising an amino acid sequence with at least 80%, 85%, 90%, 95%, 99%, or 100% sequence identity to SEQ ID NO:23. Homologs of the C. basilensis HmfT1 can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfT1 are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfF of C. basilensis or a homolog thereof. The C. basilensis HmfF is a 2,5-furandicarboxylate decarboxylase, referred to as 2,5-furandicarboxylate decarboxylase 1. 2,5-Furandicarboxylate decarboxylases catalyze the reaction: 2,5-Furandicarboxylate<=>2-Furoate+CO₂. The amino acid sequence of the C. basilensis HmfF is SEQ ID NO:26, which is encoded by SEQ ID NO:25. Homologs of the C. basilensis HmfF can include variants of the C. basilensis HmfF comprising an amino acid sequence with at least 80%, 85%, 90%, 95%, 99%, or 100% sequence identity to SEQ ID NO:26. Homologs of the C. basilensis HmfF can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfF are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfG of C. basilensis or a homolog thereof. The C. basilensis HmfG is a 2,5-furandicarboxylate decarboxylase, referred to as 2,5-furandicarboxylate decarboxylase 2. 2,5-Furandicarboxylate decarboxylases catalyze the reaction: 2,5-Furandicarboxylate<=>2-Furoate+CO₂. The amino acid sequence of the C. basilensis HmfG is SEQ ID NO:28, which is encoded by SEQ ID NO:27. Homologs of the C. basilensis HmfG can include variants of the C. basilensis HmfG comprising an amino acid sequence with at least 80%, 85%, 90%, 95%, 99%, or 100% sequence identity to SEQ ID NO:28. Homologs of the C. basilensis HmfG can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfG are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169). HmfF and HmfG are two subunits of a single enzyme. HmfF and HmfG are therefore preferably expressed together in the same recombinant microorganism, such that if the recombinant microorganism comprises a recombinant gene encoding either one of HmfF or HmfG of C. basilensis or a homolog thereof, the recombinant microorganism also preferably comprises a recombinant gene encoding the other one of HmfF or HmfG of C. basilensis or a homolog thereof.

In some versions of the invention, the one or more modifications comprise a recombinant gene encoding HmfH′ (TctC) of C. basilensis or a homolog thereof. The C. basilensis HmfH′ is a tripartite tricarboxylate transporter substrate-binding protein. The amino acid sequence of the C. basilensis HmfH′ is SEQ ID NO:30, which is encoded by SEQ ID NO:29. Homologs of the C. basilensis HmfH′ can include variants of the C. basilensis HmfH′ comprising an amino acid sequence with at least 80%, 85%, 90%, 95%, 99%, or 100% sequence identity to SEQ ID NO:30. Homologs of the C. basilensis HmfH′ can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfH′ are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfH of C. basilensis or a homolog thereof. The C. basilensis HmfH is a 5-(hydroxymethyl)furfural/furfural oxidase (EC 1.1.3.47, 1.1.3.-). 5-(Hydroxymethyl)furfural/furfural oxidases catalyze the reactions: Furfural+H₂O+Oxygen<=>2-Furoate+Hydrogen peroxide; and 5-Hydroxymethyl-2-furaldehyde+H₂O+Oxygen<=>5-Hydroxymethyl-2-furoate+Hydrogen peroxide. The amino acid sequence of the C. basilensis HmfH is SEQ ID NO:32, which is encoded by SEQ ID NO:31. Homologs of the C. basilensis HmfH can include variants of the C. basilensis HmfH comprising an amino acid sequence with at least 80%, 85%, 90%, 95%, 99%, or 100% sequence identity to SEQ ID NO:32. Homologs of the C. basilensis HmfH can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfH are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions of the invention, the one or more modifications comprise a recombinant gene encoding HmfS of C. basilensis or a homolog thereof. The C. basilensis HmfS is a fatty acid hydroxylase. Fatty acid hydroxylases add an oxygen atom to a hydrogen atom in a specific position in a fatty acid chain. They also catalyze desaturation of sterol (a lipid), during sterol biosynthesis. The amino acid sequence of the C. basilensis HmfS is SEQ ID NO:34, which is encoded by SEQ ID NO:33. Homologs of the C. basilensis HmfS can include variants of the C. basilensis HmfS comprising an amino acid sequence with at least 80%, 85%, 90%, 95%, 99%, or 100% sequence identity to SEQ ID NO:34. Homologs of the C. basilensis HmfS can also include any native homolog found in other organisms, as well as variants of such native homologs comprising an amino acid sequence with at least 80%, 85%, 90%, 95%, 99%, or 100% sequence identity thereto.

In some versions of the invention, the one or more modifications comprise a recombinant gene encoding HmfT2 of C. basilensis or a homolog thereof. The C. basilensis HmfT2 is a transporter protein in the Major Facilitator Superfamily and is related to BenE- or LivKHMGF-like transporters. The amino acid sequence of the C. basilensis HmfT2 is SEQ ID NO:36, which is encoded by SEQ ID NO:35. Homologs of the C. basilensis HmfT2 can include variants of the C. basilensis HmfT2 comprising an amino acid sequence with at least 80%, 85%, 90%, 95%, 99%, or 100% sequence identity to SEQ ID NO:36. Homologs of the C. basilensis HmfT2 can also include any native homolog found in other organisms, as well as variants of such native homologs comprising an amino acid sequence with at least 80%, 85%, 90%, 95%, 99%, or 100% sequence identity thereto. Exemplary native homologs of the C. basilensis HmfT2 are described in Donoso et al. 2021 (Donoso R A, González-Toro F, Pérez-Pantoja D. Widespread distribution of hmf genes in Proteobacteria reveals key enzymes for 5-hydroxymethylfurfural conversion. Comput Struct Biotechnol J. 2021 Apr. 16; 19:2160-2169).

In some versions, the recombinant microorganism exhibits reduced consumption of a carbohydrate, such as glucose and/or xylose, with respect to the corresponding microorganism.

In some versions, the recombinant microorganism exhibits increased consumption of a substituted furan with respect to the corresponding microorganism. Substituted furans include compounds having at least one substituent on a furan backbone:

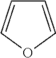

Examples of substituents include a halogen atom, alkyl, alkenyl, alkynyl, cycloalkyl, cycloalkenyl, hydroxy, carboxy, alkyloxy, alkenyloxy, alkynyloxy, cycloalkyloxy, cycloalkenyloxy, mercapto, alkylthio, alkenylthio, alkynylthio, alkylsulfinyl, alkylsulfonyl, alkylsulfonyloxy, cycloalkylthio, cycloalkylsulfinyl, cycloalkylsulfonyl, cycloalkylsulfonyloxy, cycloalkenylthio, cycloalkenylsulfinyl, cycloalkenylsulfonyl, cycloalkenylsulfonyloxy, amino, acyl, alkyloxycarbonyl, alkenyloxycarbonyl, alkynyloxycarbonyl, aryloxycarbonyl, carbamoyl, sulfamoyl, cyano, nitro, aryl, aryloxy, arylthio, arylsulfinyl, arylsulfonyl, arylsulfonyloxy, heteroaryl, heteroaryloxy, heteroarylthio, heteroarylsulfinyl, heteroarylsulfonyl, heteroarylsulfonyloxy, and non-aromatic heterocyclic. See U.S. Pat. No. 11,117,881, which is incorporated herein by reference in its entirety, for definitions of such substitutents. Specific examples of substituted furans include furfural, hydroxymethylfurfural (5-(hydroxymethyl)furfural), furfural alcohol, furoic acid, 2-methyl furan, and furfurylamine, among others. In some versions, the recombinant microorganism exhibits increased consumption of a substituted furan selected from the group consisting of furfural and hydroxymethylfurfural with respect to the corresponding microorganism. In some versions, the recombinant microorganism exhibits increased consumption of furfural with respect to the corresponding microorganism. In some versions, the recombinant microorganism exhibits increased consumption of hydroxymethylfurfural with respect to the corresponding microorganism.

The microorganisms of the invention may be genetically altered to functionally delete, express, or overexpress homologs of any of the specific genes or gene products explicitly described herein. Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Nucleic acid or gene product (amino acid) sequences of any known gene, including the genes or gene products described herein, can be determined by searching any sequence databases known the art using the gene name or accession number as a search term. Common sequence databases include GenBank (ncbi.nlm.nih.gov/genbank/), ExPASy (expasy.org), KEGG (genome.jp/kegg/), among others. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 35% 40%, 45% 50%, 55%, 60%, 65%, 70%, 75%, 80%, 85%, 90%, 95%, or 99% or more, can also be used to establish homology. Accordingly, homologs of the genes or gene products described herein include genes or gene products having at least about 30%, 35%, 40%, 45%, 50%, 55%, 60%, 65%, 70%, 75%, 80%, 85%, 90%, 95%, or 99% identity to the genes or gene products described herein. Methods for determining sequence similarity percentages (e.g., BLASTP and BLASTN using default parameters) are described herein and are generally available. The homologous proteins should demonstrate comparable activities and, if an enzyme, participate in the same or analogous pathways. “Orthologs” are genes in different species that evolved from a common ancestral gene by speciation. Normally, orthologs retain the same or similar function in the course of evolution. As used herein “orthologs” are included in the term “homologs”.

For sequence comparison and homology determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is a nucleic acid or amino acid sequence corresponding to acsA or other genes or products described herein.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described above (or other algorithms available to persons of skill) or by visual inspection.

The phrase “substantially identical” in the context of two nucleic acids or polypeptides refers to two or more sequences or subsequences that have at least about 60%, about 65%, about 70%, about 75%, about 80%, about 85%, about 90, about 95%, about 98%, or about 99% or more nucleotide or amino acid residue identity, when compared and aligned for maximum correspondence, as measured using a sequence comparison algorithm or by visual inspection. Such “substantially identical” sequences are typically considered to be “homologous”, without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, at least about 250 residues, or over the full length of the two sequences to be compared.

Terms used herein pertaining to genetic manipulation are defined as follows.

Deletion: The removal of one or more nucleotides from a nucleic acid molecule or one or more amino acids from a protein, the regions on either side being joined together.

Derived: When used with reference to a nucleic acid or protein, “derived” means that the nucleic acid or polypeptide is isolated from a described source or is at least 70%, 80%, 90%, 95%, 99%, or more identical to a nucleic acid or polypeptide included in the described source.

Endogenous: An endogenous nucleic acid, gene, gene element (e.g., promoter, enhancer, coding sequence), polypeptide, sequence or any other element in a given cell is one that is naturally occurring in the given cell.

Exogenous: An exogenous nucleic acid, gene, gene element (e.g., promoter, enhancer, coding sequence), polypeptide, sequence or any other element in a given cell is one that is not naturally occurring in the given cell. The term “heterologous” is used herein interchangeably with “exogenous.”

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Gene: “Gene” refers minimally to a coding sequence and a promoter operationally linked to the coding sequence. A gene may additionally include other elements, such as enhancers and silencers.

Introduce: When used with reference to genetic material, such as a nucleic acid, and a cell, “introduce” refers to the delivery of the genetic material to the cell in a manner such that the genetic material is capable of being expressed within the cell. Introduction of genetic material includes both transformation and transfection. Transformation encompasses techniques by which a nucleic acid molecule can be introduced into cells such as prokaryotic cells or non-animal eukaryotic cells. Transfection encompasses techniques by which a nucleic acid molecule can be introduced into cells such as animal cells. These techniques include but are not limited to introduction of a nucleic acid via conjugation, electroporation, lipofection, infection, and particle gun acceleration.

Isolated: An “isolated” biological component (such as a nucleic acid molecule, polypeptide, or cell) has been substantially separated or purified away from other biological components in its original form, such as its native form or the form in which it was originally produced.

Nucleic acid: Encompasses both RNA and DNA molecules including, without limitation, cDNA, genomic DNA, and mRNA. Nucleic acids also include synthetic nucleic acid molecules, such as those that are chemically synthesized or recombinantly produced. The nucleic acid can be double-stranded or single-stranded. Where single-stranded, the nucleic acid molecule can be the sense strand, the antisense strand, or both. In addition, the nucleic acid can be circular or linear.

Operably linked: A first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. An origin of replication is operably linked to a coding sequence if the origin of replication controls the replication or copy number of the nucleic acid in the cell. Operably linked nucleic acids may or may not be contiguous.

Operon: Configurations of separate genes that are transcribed in tandem as a single messenger RNA are denoted as operons. Thus, a set of in-frame genes in close proximity under the transcriptional regulation of a single promoter constitutes an operon. Operons may be synthetically generated using the methods described herein.

Overexpress: When a gene is caused to be transcribed at an elevated rate compared to the endogenous or basal transcription rate for that gene. In some examples, overexpression additionally includes an elevated rate of translation of the gene compared to the endogenous translation rate for that gene. Methods of testing for overexpression are well known in the art, for example transcribed RNA levels can be assessed using rtPCR and protein levels can be assessed using SDS page gel analysis.

Recombinant: A recombinant nucleic acid, gene, gene element (e.g., promoter, enhancer, coding sequence), or polypeptide is one that has a sequence that is not naturally occurring. A recombinant cell or microorganism is one that contains a recombinant nucleic acid, gene, gene element (e.g., promoter, enhancer, coding sequence), or polypeptide.

Vector or expression vector: An entity comprising a nucleic acid molecule that is capable of introducing the nucleic acid, or being introduced with the nucleic acid, into a cell for expression of the nucleic acid. A vector can include nucleic acid sequences that permit it to replicate in the cell, such as an origin of replication. A vector can also include one or more selectable marker genes and other genetic elements known in the art. Examples of suitable vectors are found below.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below.

Exogenous nucleic acids can be introduced stably or transiently into a cell using techniques well known in the art, including electroporation, calcium phosphate precipitation, DEAE-dextran mediated transfection, liposome-mediated transfection, conjugation, transduction, and the like. For stable transformation, a nucleic acid can further include a selectable marker. Suitable selectable markers include antibiotic resistance genes that confer, for example, resistance to neomycin, tetracycline, chloramphenicol, or kanamycin, genes that complement auxotrophic deficiencies, and the like. (See below for more detail.)

Various embodiments of the invention use an expression vector that includes a heterologous nucleic acid encoding a protein. Suitable expression vectors include, but are not limited to viral vectors, such as baculovirus vectors or those based on vaccinia virus, polio virus, adenovirus, adeno-associated virus, SV40, herpes simplex virus, and the like; phage vectors, such as bacteriophage vectors; plasmids; phagemids; cosmids; fosmids; bacterial artificial chromosomes; P1-based artificial chromosomes; yeast plasmids; yeast artificial chromosomes; and any other vectors specific for cells of interest.

Useful vectors can include one or more selectable marker genes to provide a phenotypic trait for selection of transformed cells. The selectable marker gene encodes a protein necessary for the survival or growth of transformed cells grown in a selective culture medium. Cells not transformed with the vector containing the selectable marker gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. In alternative embodiments, the selectable marker gene is one that encodes dihydrofolate reductase or confers neomycin resistance (for use in eukaryotic cell culture), or one that confers tetracycline or ampicillin resistance (for use in a prokaryotic cell, such as E. coli).

The coding sequence in the expression vector is operably linked to an appropriate expression control sequence (promoters, enhancers, and the like) to direct synthesis of the encoded gene product. Such promoters can be derived from microbial or viral sources, including CMV and SV40. Depending on the cell/vector system utilized, any of a number of suitable transcription and translation control elements, including constitutive and inducible promoters, transcription enhancer elements, transcription terminators, etc. can be used in the expression vector (see e.g., Bitter et al. (1987) Methods in Enzymology, 153:516-544).

Suitable promoters for use in prokaryotic cells include but are not limited to: promoters capable of recognizing the T4, T3, Sp6, and T7 polymerases; the P_{R} and P_{L} promoters of bacteriophage lambda; the trp, recA, heat shock, and lacZ promoters of E. coli; the alpha-amylase and the sigma-specific promoters of B. subtilis; the promoters of the bacteriophages of Bacillus; Streptomyces promoters; the int promoter of bacteriophage lambda; the bla promoter of the beta-lactamase gene of pBR322; and the CAT promoter of the chloramphenicol acetyl transferase gene. Prokaryotic promoters are reviewed by Glick, J. Ind. Microbiol. 1:277 (1987); Watson et al, Molecular Biology of the Gene, 4th Ed., Benjamin Cummins (1987); and Sambrook et al., In: Molecular Cloning: A Laboratory Manual, 3^{rd} ed., Cold Spring Harbor Laboratory Press (2001).

Non-limiting examples of suitable promoters for use within a eukaryotic cell are typically viral in origin and include the promoter of the mouse metallothionein I gene (Hamer et al. (1982) J. Mol. Appl. Gen. 1:273); the TK promoter of Herpes virus (McKnight (1982) Cell 31:355); the SV40 early promoter (Benoist et al. (1981) Nature (London) 290:304); the Rous sarcoma virus promoter; the cytomegalovirus promoter (Foecking et al. (1980) Gene 45:101); the yeast gal4 gene promoter (Johnston et al. (1982) PNAS (USA) 79:6971; Silver et al. (1984) PNAS (USA) 81:5951); and the IgG promoter (Orlandi et al. (1989) PNAS (USA) 86:3833).

Coding sequences can be operably linked to an inducible promoter. Inducible promoters are those wherein addition of an effector induces expression. Suitable effectors include proteins, metabolites, chemicals, or culture conditions capable of inducing expression. Suitable inducible promoters include but are not limited to the lac promoter (regulated by IPTG or analogs thereof), the lacUV5 promoter (regulated by IPTG or analogs thereof), the tac promoter (regulated by IPTG or analogs thereof), the trc promoter (regulated by IPTG or analogs thereof), the araBAD promoter (regulated by L-arabinose), the phoA promoter (regulated by phosphate starvation), the recA promoter (regulated by nalidixic acid), the proU promoter (regulated by osmolarity changes), the cst-1 promoter (regulated by glucose starvation), the tetA promoter (regulated by tetracycline), the cadA promoter (regulated by pH), the nar promoter (regulated by anaerobic conditions), the p_{L} promoter (regulated by thermal shift), the cspA promoter (regulated by thermal shift), the T7 promoter (regulated by thermal shift), the T7-lac promoter (regulated by IPTG), the T3-lac promoter (regulated by IPTG), the T5-lac promoter (regulated by IPTG), the T4 gene 32 promoter (regulated by T4 infection), the nprM-lac promoter (regulated by IPTG), the VHb promoter (regulated by oxygen), the metallothionein promoter (regulated by heavy metals), the MMTV promoter (regulated by steroids such as dexamethasone) and variants thereof.

Alternatively, a coding sequence can be operably linked to a repressible promoter. Repressible promoters are those wherein addition of an effector represses expression. Examples of repressible promoters include but are not limited to the trp promoter (regulated by tryptophan); tetracycline-repressible promoters, such as those employed in the “TET-OFF”-brand system (Clontech, Mountain View, CA); and variants thereof.

In some versions, the cell is genetically modified with a heterologous nucleic acid encoding a gene product that is operably linked to a constitutive promoter. Suitable constitutive promoters are known in the art and include constitutive adenovirus major late promoter, a constitutive MPSV promoter, and a constitutive CMV promoter.

The relative strengths of the promoters described herein are well-known in the art.

In some versions, the cell is genetically modified with an exogenous nucleic acid encoding a single protein. In other embodiments, a modified cell is one that is genetically modified with exogenous nucleic acids encoding two or more proteins. Where the cell is genetically modified to express two or more proteins, those nucleic acids can each be contained in a single or in separate expression vectors. When the nucleic acids are contained in a single expression vector, the nucleotide sequences may be operably linked to a common control element (e.g., a promoter), that is, the common control element controls expression of all of the coding sequences in the single expression vector.

When the cell is genetically modified with heterologous nucleic acids encoding two or more proteins, one of the nucleic acids can be operably linked to an inducible promoter, and one or more of the nucleic acids can be operably linked to a constitutive promoter. Alternatively, all can be operably linked to inducible promoters or all can be operably linked to constitutive promoters.

Nucleic acids encoding enzymes desired to be expressed in a cell may be codon-optimized for that particular type of cell. Codon optimization can be performed for any nucleic acid by “OPTIMUMGENE”-brand gene design system by GenScript (Piscataway, NJ).

The introduction of a vector into a bacterial cell may be performed by protoplast transformation (Chang and Cohen (1979) Molecular General Genetics, 168:111-115), using competent cells (Young and Spizizen (1961) Journal of Bacteriology, 81:823-829; Dubnau and Davidoff-Abelson (1971) Journal of Molecular Biology, 56: 209-221), electroporation (Shigekawa and Dower (1988) Biotechniques, 6:742-751), or conjugation (Koehler and Thorne (1987) Journal of Bacteriology, 169:5771-5278). Commercially available vectors for expressing heterologous proteins in bacterial cells include but are not limited to pZERO, pTrc99A, pUC19, pUC18, pKK223-3, pEXI, pCAL, pET, pSPUTK, pTrxFus, pFastBac, pThioHis, pTrcHis, pTrcHis2, and pLEx, in addition to those described in the following Examples.

Methods for transforming yeast cells with heterologous DNA and producing heterologous polypeptides therefrom are disclosed by Clontech Laboratories, Inc., Palo Alto, Calif, USA (in the product protocol for the “YEASTMAKER”-brand yeast transformation system kit); Reeves et al. (1992) FEMS Microbiology Letters 99:193-198; Manivasakam and Schiestl (1993) Nucleic Acids Research 21(18):4414-5; and Ganeva et al. (1994) FEMS Microbiology Letters 121:159-64. Expression and transformation vectors for transformation into many yeast strains are available. For example, expression vectors have been developed for the following yeasts: Candida albicans (Kurtz, et al. (1986) Mol. Cell. Biol. 6:142); Candida maltosa (Kunze et al. (1985) J. Basic Microbiol. 25:141); Hansenula polymorpha (Gleeson et al. (1986) J. Gen. Microbiol. 132:3459) and Roggenkamp et al. (1986) Mol. Gen. Genet. 202:302); Kluyveromyces fragilis (Das et al. (1984) J. Bacteriol. 158:1165); Kluyveromyces lactis (De Louvencourt et al. (1983) J. Bacteriol. 154:737) and Van den Berg et al. (1990) Bio/Technology 8:135); Pichia quillerimondii (Kunze et al. (1985) J. Basic Microbiol. 25:141); Pichia pastoris (Cregg et al. (1985) Mol. Cell. Biol. 5:3376; U.S. Pat. Nos. 4,837,148; and 4,929,555); Saccharomyces cerevisiae (Hinnen et al. (1978) Proc. Natl. Acad. Sci. USA 75:1929 and Ito et al. (1983) J. Bacteriol. 153:163); Schizosaccharomyces pombe (Beach et al. (1981) Nature 300:706); and Yarrowia lipolytica (Davidow et al. (1985) Curr. Genet. 10:380-471 and Gaillardin et al. (1985) Curr. Genet. 10:49).

Suitable procedures for transformation of Aspergillus cells are described in EP 238 023 and U.S. Pat. No. 5,679,543. Suitable methods for transforming Fusarium species are described by Malardier et al., Gene, 1989, 78:147-56 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al. (1983) Journal of Bacteriology, 153: 163; and Hinnen et al. (1978) PNAS USA, 75:1920.

Another aspect of the invention is directed to methods of decreasing an amount of a substituted furan in a medium. The methods can comprise contacting the medium with a recombinant microorganism of the invention for a time sufficient to decrease the furan in the medium. In some versions, the furan is selected from the group consisting of furfural and hydroxymethylfurfural, such that the methods comprise contacting the medium with a recombinant microorganism of the invention for a time sufficient to decrease furfural, hydroxymethylfurfural, or furfural and hydroxymethylfurfural in the medium. In some versions, the medium further comprises a carbohydrate, such as glucose and/or xylose.

In various versions of the invention, the medium is contacted with a recombinant microorganism of the invention for a time sufficient to decrease the furan in the medium to an amount by mass less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, less than 1%, or 0% of an amount by mass of the furan present in the medium prior to contacting.

In various versions of the invention, the medium is contacted with a recombinant microorganism of the invention for a time sufficient to decrease furfural in the medium to an amount by mass less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, less than 1%, or 0% of an amount by mass of the furfural present in the medium prior to contacting.

In various versions of the invention, the medium is contacted with a recombinant microorganism of the invention for a time sufficient to decrease hydroxymethylfurfural in the medium to an amount by mass less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, less than 1%, or 0% of an amount by mass of the hydroxymethylfurfural present in the medium prior to contacting.

In various versions of the invention, glucose is maintained over the entire course of the time in which the medium is contacted with the recombinant microorganism in an amount by mass of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% of an amount by mass of the glucose present in the medium prior to contacting.

In various versions of the invention, xylose is maintained over the entire course of the time in which the medium is contacted with the recombinant microorganism in an amount by mass of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or 100% of an amount by mass of the xylose present in the medium prior to contacting.

In various versions of the invention, the medium is contacted with a recombinant microorganism of the invention for a time of at least 0.5 hours, at least 1 hour, at least 1.5 hours, at least 2 hours, or more. In various versions of the invention, the medium is contacted with a recombinant microorganism of the invention for a time up to 1 hour, up to 1.5 hours, up to 2 hours, up to 2.5 hours, up to 3 hours, up to 3.5 hours, up to 4 hours, up to 4.5 hours, up to 5 hours, up to 5.5 hours, up to 6 hours, up to 6.5 hours, up to 7 hours, up to 7.5 hours, up to 8 hours, up to 8.5 hours, up to 9 hours, up to 9.5 hours, up to 10 hours, up to 15 hours, up to 20 hours, up to 25 hours, up to 30 hours, or more.

In some versions of the invention, the contacting is performed under aerobic conditions. “Aerobic conditions” refers to the presence of free oxygen (O₂), such as in a gas contacting the medium. In some versions, the gas comprises at least 1 vol % free oxygen, at least 5 vol % free oxygen, at least 10 vol % free oxygen, at least 15 vol % free oxygen, or at least 20 vol % free oxygen. In some versions, the gas comprises up to 25 vol % free oxygen, up to 30 vol % free oxygen, up to 35 vol % free oxygen, up to 40 vol % free oxygen, up to 45 vol % free oxygen, up to 50 vol % free oxygen, up to 55 vol % free oxygen, up to 60 vol % free oxygen, up to 65 vol % free oxygen, up to 70 vol % free oxygen, up to 75 vol % free oxygen, up to 80 vol % free oxygen, up to 85 vol % free oxygen, up to 90 vol % free oxygen, up to 95 vol % free oxygen, up to 99 vol % free oxygen, or about 100 vol % free oxygen.

In some versions, the medium comprises lignocellulosic biomass. The lignocellulosic biomass can be derived from any source, such as corn cobs, corn stover, cotton seed hairs, grasses, hardwood stems, leaves, newspaper, nut shells, paper, softwood stems, sorghum, switchgrass, waste papers from chemical pulps, wheat straw, wood, woody residues, mixed biomass species such as those produced by native prairie, and other sources.

The lignocellulosic biomass is preferably processed lignocellulosic biomass. “Processed lignocellulosic biomass” refers to lignocellulosic biomass that has been chemically or physically processed. Various methods of processing lignocellulosic biomass are known in the art. See Pandey et al. 2010 (Pandey M P, Kim C S. Lignin Depolymerization and Conversion: A Review of Thermochemical Methods. Chemical & Engineering Technology, 2010, Vol. 34, Issue 1, pp. 3-145), Wang et al. 2013 (Wang H, Tucker M, Ji Y. Recent Development in Chemical Depolymerization of Lignin: A Review. Journal of Applied Chemistry, 2013, Volume 2013, Article ID 838645), Kumar et al. 2017 (Kumar AK and Sharma S. Recent Updates on Different Methods of Pretreatment of Lignocellulosic Feedstocks: A Review. Bioresour. Bioprocess. (2017) 4:7), Kumar et al. 2009 (Kumar, P.; Barrett, D. M.; Delwiche, M. J.; Stroeve, P., Methods for Pretreatment of lignocellulosic Biomass for Efficient Hydrolysis and Biofuel Production. Industrial & Engineering Chemistry Research 2009, 48, (8), 3713-3729), Wang et al. 2013 (Wang H, Tucker M, Ji Y. Recent Development in Chemical Depolymerization of Lignin: A Review. (2013) Journal of Applied Chemistry. 2013:1-9), Karlen et al. 2020 (Karlen S D, Fasahati P, Mazaheri M, Serate J, Smith R A, Sirobhushanam S, Chen M, Tymkhin V I, Cass C L, Liu S, Padmakshan D, Xie D, Zhang Y, McGee M A, Russell J D, Coon J J, Kaeppler H F, de Leon N, Maravelias C T, Runge T M, Kaeppler S M, Sedbrook J C, Ralph J. Assessing the viability of recovering hydroxycinnamic acids from lignocellulosic biorefinery alkaline pretreatment waste streams. ChemSusChem. 2020 Jan. 26), and Jönsson et al. 2013 (Jönsson LJ, Alriksson B, Nilvebrant NO. Bioconversion of lignocellulose: inhibitors and detoxification. Biotechnol Biofuels. 2013 Jan. 28; 6(1):16). Examples of lignocellulosic biomass processing include chipping, grinding, milling, steam pretreatment, ammonia fiber expansion (AFEX, also referred to as ammonia fiber explosion), ammonia recycle percolation (ARP), CO₂ explosion, steam explosion, ozonolysis, wet oxidation, acid hydrolysis, dilute-acid hydrolysis, alkaline hydrolysis, organosolv, ionic liquids, gamma-valerolactone, and pulsed electrical field treatment, among others.

In some versions, the medium comprises lignocellulosic biomass hydrolysate. Lignocellulosic biomass hydrolysates are processed forms of lignocellulosic biomass that have undergone hydrolytic processing, such as through enzymatic hydrolysis, acid hydrolysis, dilute-acid hydrolysis, and alkaline hydrolysis, among others.

Some methods of the invention the contacting the medium with a recombinant microorganism of the invention generates a second medium and the methods further comprise, after contacting the medium with a recombinant microorganism of the invention, fermenting the second medium with a second microorganism. In some versions, the second microorganism is not the recombinant microorganism of the invention. In some versions, the second microorganism is an anaerobe or a facultative anaerobe. In some versions, the fermenting is performed under anaerobic conditions. In some versions, the fermenting consumes glucose and/or xylose in the second medium. In some versions, the fermenting converts the glucose and/or xylose to ethanol or other compounds.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

An Engineered Strain of Pseudomonas putida for Dedicated Selective Detoxification of Lignocellulosic Hydrolysates.

Despite their enormous potential as economical and renewable feedstocks for the production of biofuels and biochemicals, lignocellulosic biomasses (LB) are still severely plagued by a formidable technical issue. Specifically, deconstruction of LB by acid- or alkali-based pretreatment to obtain fermentable sugars is a cost-effective approach to release sugars from LB. However, the release of fermentable sugars from LB by this approach is accompanied by co-generation of furanic and phenolic aldehydes that exert severe toxicity on fermenting microorganisms. Consequently, economical decontamination of LB hydrolysates (LBHs) is a critical prerequisite to bioconversion of LB to value-added chemicals. Although bioabatement is recognized as a potential practical and economical strategy to overcome this challenge, the vast majority of efforts towards this goal have focused on metabolic engineering of single strains that can efficiently decontaminate LBH-borne inhibitory aldehydes and simultaneously produce target chemicals. Despite considerable progress, this approach has failed thus far to generate strains capable of these tasks (i.e., simultaneous decontamination and target chemical production).

Additionally, most fermentative processes that convert sugars to value-added chemicals are anaerobic. Under this condition, the furanic and phenolic aldehydes are merely reduced to their less toxic alcohols. While this minimizes the toxicity of LBHs, it does not eliminate it entirely, as the resulting alcohols (following the reduction of toxic aldehydes to alcohols) and residual aldehydes conspire to retain toxicity on fermenting cells. Consequently, bioproduction of target chemicals is greatly diminished, thereby derailing commercialization efforts.

The present examples provide the foundation for a different, two-step strategy for the valorization of LBH. In a first step, a dedicated strain is engineered to solely decontaminate LBH-borne inhibitors pre-fermentation. Instead of reducing the inhibitory aldehydes to their corresponding alcohols, the engineered strain described herein is intended to catabolize and thereby completely remove the inhibitors from the medium pre-fermentation (aerobically). In the second step, the inhibitor-free LBH will be seamlessly fermented to any target chemical with an appropriate microorganism.

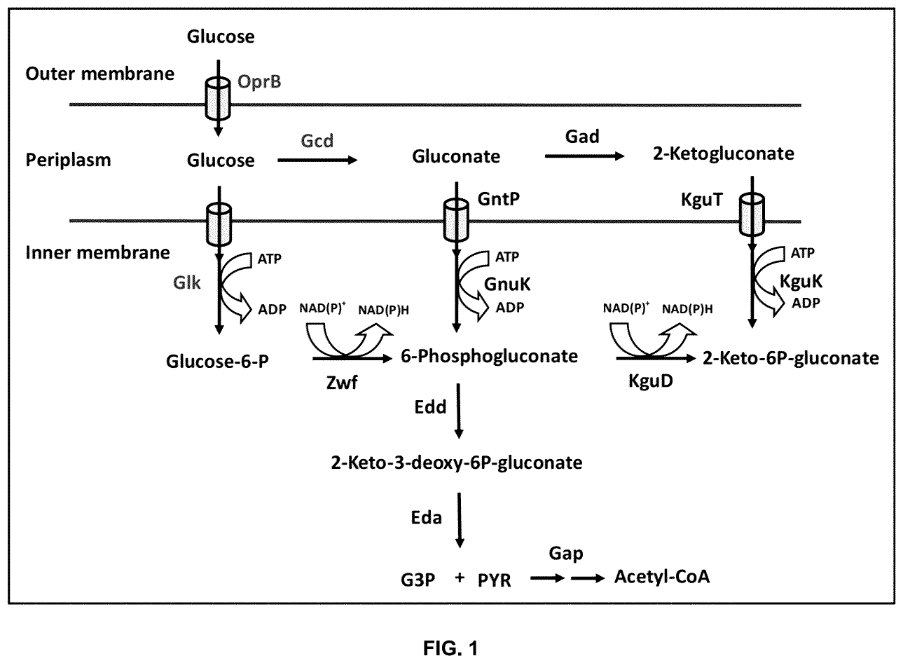

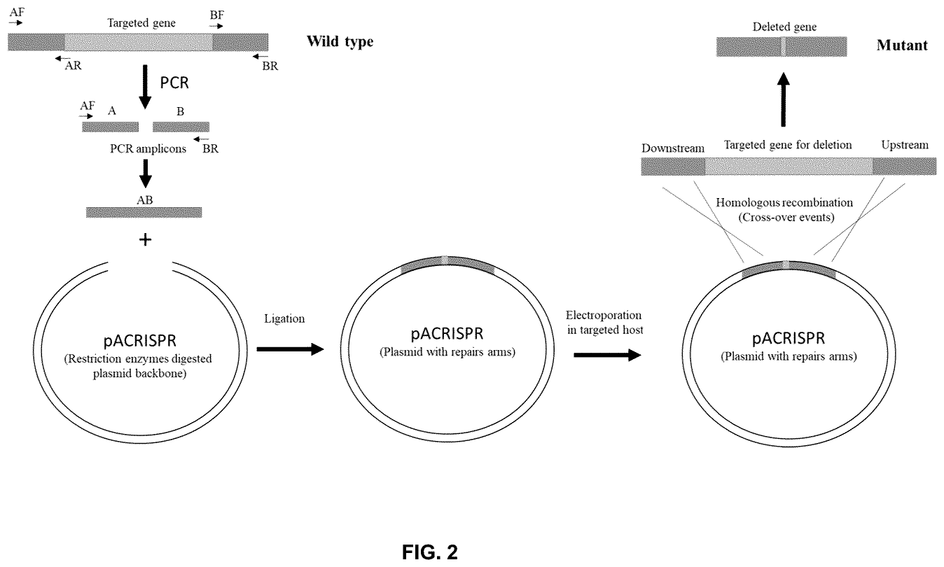

A critical step towards engineering a dedicated strain that selectively utilizes LBH-borne inhibitors as carbon sources—without sugar utilization—is to eliminate the sugar utilization machinery of the organism. That way, the resulting strain exclusively utilizes the inhibitors as carbon sources, and in so doing, eliminate them from the LBH. To this end, Pseudomonas putida DSM 6125 was selected as a base microbe for engineering. This strain utilizes glucose but is incapable of xylose utilization. Xylose is the second most abundant sugar in LBHs. A triple mutant (oprB-II⁻/gcd⁻/glk⁻) of P. putida DSM 6125 has been engineered by eliminating the glucose import permease gene (oprB-II), glucose dehydrogenase gene (gcd), and glucokinase gene (glk) (FIGS. 1 and 2). Alternative strains include Escherichia coli and Enterobacter hormaechei, from which the glucose dehydrogenase gene (gcd) and glucokinase gene (glk) can be deleted.

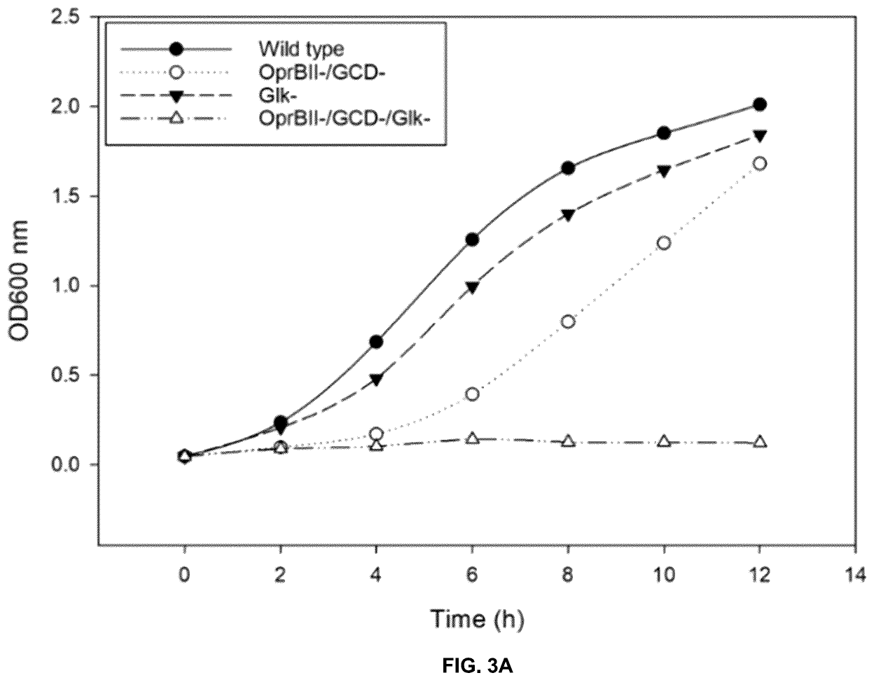

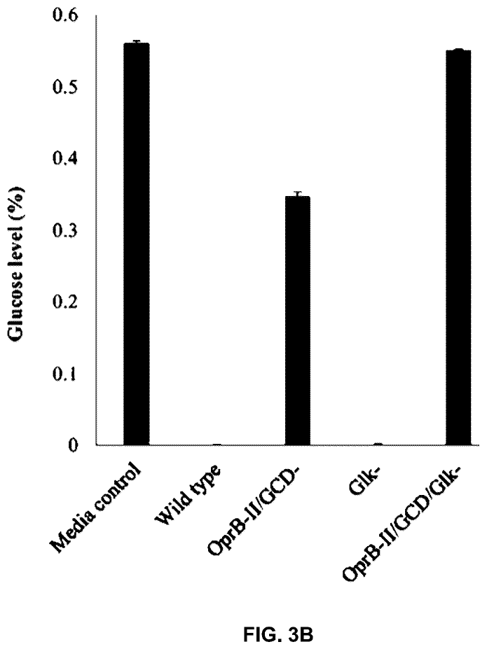

The growth profiles of wildtype P. putida DSM 6125, the triple deletion mutant (P. putida_oprB-II⁻/gcd⁻/glk⁻), the single deletion mutant (P. putida_glk⁻) and the double deletion mutant (P. putida_oprB-II⁻/gcd⁻) confirm complete elimination of the glucose utilization machinery of this organism in the triple deletion mutant (FIGS. 3A and 3B). Whereas the wildtype and P. putida_glk⁻ completely consumed the glucose in a glucose medium (FIG. 3B), P. putida_oprB-II⁻/gcd⁻ consumed only 58% of the glucose in the medium (FIG. 3B), and P. putida_oprB-II⁻/gcd⁻/glk⁻ did not grow in the glucose medium (FIG. 3A). Additionally, there was no change in glucose concentration in cultures of P. putida_oprB-II⁻/gcd⁻/glk⁻ (FIG. 3B).

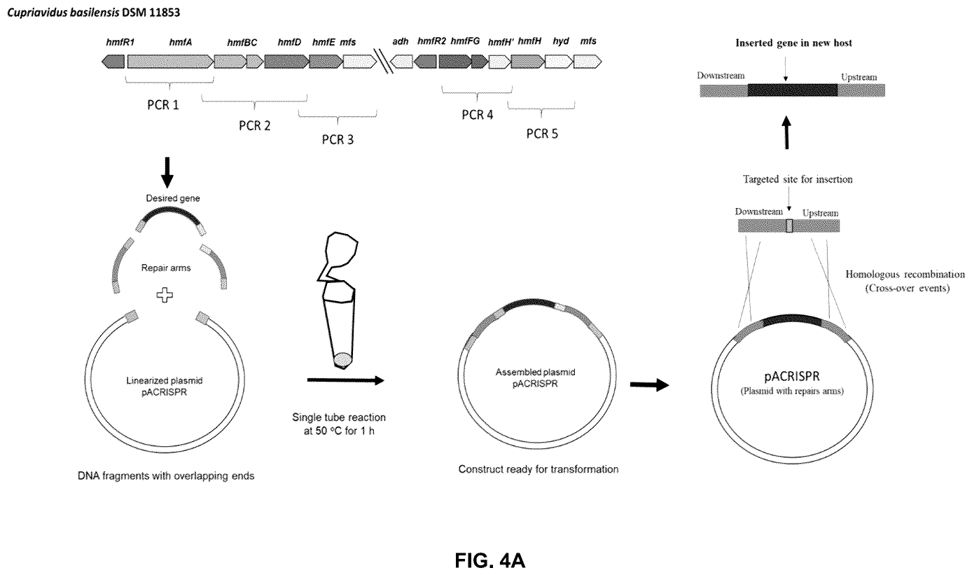

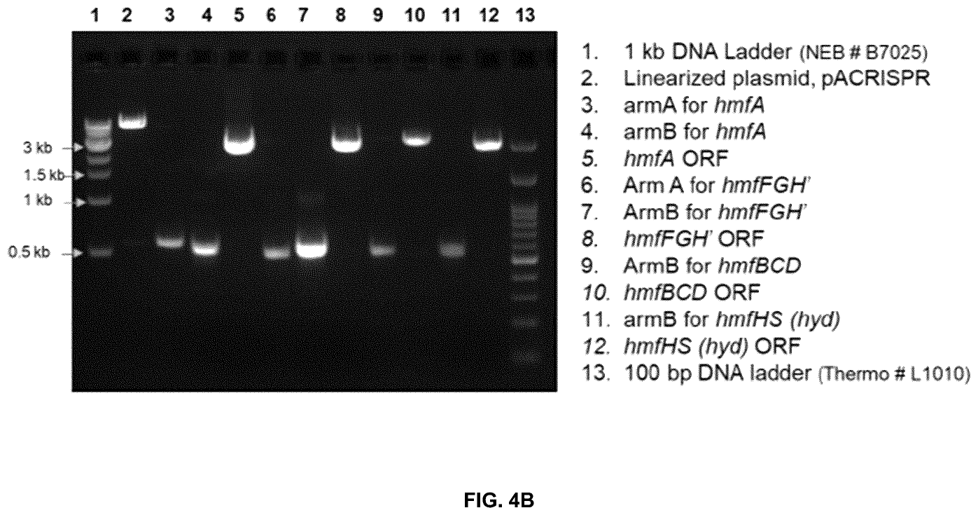

Having eliminated glucose utilization in an organism that does not utilize xylose, our next task is to amplify the capacity of P. putida_oprB-II⁻/gcd⁻/glk⁻ to utilize LBH-borne inhibitors, particularly, furfural and 5-hydroxymethyl furfural (HMF), the two most abundant and most toxic inhibitors in LBHs. This will ensure rapid catabolism of inhibitors, hence, removal from LBHs pre-fermentation. Although P. putida DSM 6125 utilizes furfural and HMF as carbon sources, it does so slowly and at low concentrations (1-2 g/L). However, LBHs contain as high as 6 g/L furfural and 3 g/L HMF. Therefore, to expedite furfural and HMF catabolism and, thus, rapid detoxification of LBHs, genetic elements for rapid inhibitor transport and utilization are currently being integrated into P. putida_oprB-II⁻/gcd⁻/glk⁻. Genes encoding inhibitor transporters and catabolic enzymes have been amplified from Cupriavidus basilensis DSM 11853 (FIGS. 4A and 4B), which is capable of excellent utilization of furfural and HMF (as well as phenolic inhibitors).

The furfural metabolizing gene cluster (from C. basilensis DSM 11853) containing the genes hmfABCDET1 (SEQ ID NO:11) and the cluster involved in the metabolism of HMF to 2-furoic acid containing the genes hmfFGH′HST2 (SEQ ID NO:24) have been sequentially amplified by PCR and assembled (using Gibson assembly) for integration into P. putida_oprB-II/gcd⁻/glk⁻ (FIGS. 4A and 4B). These operons include genes for major facilitator superfamily transporters (MFS; hmfT1, hmfT2), which aid the import of furans into the cell. Plasmids harboring desired genes with their repair arms have been electroporated into P. putida for the first round to integrate hmfFGH′ into the genome at the glk⁻ deletion site (in-frame to translation start codon of glk) in P. putida_oprB-II⁻/gcd⁻/glk⁻ using the homologous recombination. The resulting colonies are currently being screened for successful integration. After confirmation of successful integration of hmfFGH′, additional plasmid constructs with hmfHS and hmfT2 will be integrated next to hmfFGH′. Similarly, genes for furfural metabolism (hmfABCDET1) will be integrated at the gcd⁻ site in P. putida. After complete insertion of genes of both hmf clusters of C. basilensis into P. putida_oprB-II⁻/gcd⁻/glk⁻, the growth of engineered strain will be evaluated on furfural and HMF as sole carbon sources in mineral media and then in LBHs.

TABLE 1
Genes for Deleting in Pseudomonas putida DSM 6125.

| Gene/ | | | | SEQ ID NOS |
|---|---|---|---|---|
| enzyme name | Gene symbol | E.C. number | Function | (NT, PRT) |
| Glucokinase | glk | [EC: 2.7.1.2] | Carbohydrate | 1, 2 |
| | | | metabolism | |
| Quinoprotein | gcd | [EC: 1.1.5.2] | Carbohydrate | 3, 4 |
| glucose | | | metabolism | |
| dehydrogenase | | | | |
| Carbohydrate- | oprB-II | n/a | Transporter | 5, 6 |
| selective porin | | | | |

TABLE 2
Genes for Deleting in Escherichia coli
and Enterobacter hormaechei.

| Gene/enzyme | Gene | | | SEQ ID NOS* |
|---|---|---|---|---|
| name | symbol | E.C. number | Function | (NT, PRT) |
| Glucokinase | glk | [EC: 2.7.1.2] | Carbohydrate | 7, 8 |
| | | | metabolism | |
| Quinoprotein | gcd | [EC: 1.1.5.2] | Carbohydrate | 9, 10 |
| glucose | | | metabolism | |
| dehydrogenase | | | | |

*SEQ ID NOS are provided for Enterobacter hormaechei

TABLE 3
Genes for Insertion (e.g., in Pseudomonas putida DSM
6125, Escherichia coli, or Enterobacter hormaechei)

| | | | | |
|---|---|---|---|---|
| | | | | SEQ ID NOS |
| Gene/enzyme name | Gene symbol | E.C. number | Function | (NT, PRT) |

HMF-ABCDET operon (furfural utilizing genes) (SEQ ID NO: 11)

| | | | | |
|---|---|---|---|---|
| Molybdopterin- | hmfA | [EC: 1.3.99.8] | Aerobic-type | 12, 13 |
| dependent | | | carbon | |
| oxidoreductase | | | monoxide | |
| | | | dehydrogenase | |
| | | | homologue | |
| FAD binding | hmfB | [EC: 1.3.99.8] | Carbon- | 14, 15 |
| domain-containing | | | monoxide | |
| protein | | | dehydrogenase | |
| (2Fe—2S)-binding | hmfC | n/a | Aerobic-type | 16, 17 |
| protein | | | carbon | |
| | | | monoxide | |
| | | | dehydrogenase | |
| | | | 2Fe—2S iron- | |
| | | | sulfur subunit | |
| AMP-binding | hmfD | [EC: 6.2.1.31] | Furoyl-CoA | 18, 19 |
| protein | | | syntethase | |
| Enoyl-CoA | hmfE | n/a | 2-oxoglutaroyl- | 20, 21 |
| hydratase/isomerase | | | CoA hydrolase | |
| family protein | | | | |
| MFS transporter | hmfT1 | n/a | Transport | 22, 23 |

HMF-FGH′HST′ operon (5-HMF utilizing genes) (SEQ ID NO: 24)

| | | | | |
|---|---|---|---|---|
| UbiD family | hmfF | [EC: 4.1.1.98] | 2,5-furan- | 25, 26 |
| decarboxylase | | | dicarboxylic | |
| | | | acid | |
| | | | decarboxylase 1 | |
| UbiX family flavin | hmfG | [EC: 2.5.1.129] | 2,5-furan- | 27, 28 |
| prenyltransferase | | | dicarboxylic | |
| | | | acid | |
| | | | decarboxylase 2 | |
| Tripartite | hmfH′ (tctC) | n/a | transport | 29, 30 |
| tricarboxylate | | | | |
| transporter | | | | |
| substrate-binding | | | | |
| protein | | | | |
| GMC family | hmfH | [EC: 1.1.3.47] | HMF/furfural | 31, 32 |
| oxidoreductase | | | oxidoreductase | |
| Sterol desaturase | hmfS | n/a | fatty acid | 33, 34 |
| family protein | | | hydroxylase | |
| MFS transporter | hmfT2 | n/a | transport | 35, 36 |

### Methodology of Gene Deletion and Integration

For in-frame deletion of target genes (Glucokinase (glk), glucose dehydrogenase (gcd), and carbohydrate-selective porin (oprB-HI)), upstream and downstream of the target genes (repair arms of approx. 550 bp) were amplified separately (as fragment A & B) using two sets of primers (named as AF, AR & BF, BR; see sequences below) and gDNA of P. putida by standard PCR method (98° C. for 3 min, 35 cycles of (50-55° C. for 30 sec, 72° C. for 1 min) and final extension of 72° C. for 5 min and 4° C. hold). Both sets of primers were designed to have appropriate restriction sites in the external primers (XhoI on AF and HindIII on BR), and overlapping bases in the internal primers (AR and BF) so that amplified fragments (A and B) could be fused together using overlapping PCR (98° C. for 3 min, 15 cycles of (50° C. for 5 min, 72° C. for 1 min), and final extension of 72° C. for 5 min and 4° C. hold). A fused amplicon (AB) was subsequently cloned by restriction digestion and ligation into a modified pACRISPR^{s} plasmid digested similarly with restriction enzymes (XhoI and HindIII) to create a recombinant plasmid (pACRISPR^{s}-AB) carrying SacB and gentamicin (GenR) cassettes for selection on sucrose and gentamicin respectively. The ligated product was transformed into DH5a cells and selected on gentamicin plates (50 μg/mL). Gentamicin-resistant colonies were then screened for fragment-AB by colony PCR using an end set of primers (AF and BR). Positive clones were processed for plasmid isolation and further confirmed by sequencing. To delete target genes, pACRISPR^{s}-AB was transferred by electroporation into electrocompetent cells of P. putida DSM 6125 and transformed colonies were selected on a gentamicin antibiotic plate (25 μg/mL). Only a few colonies were found growing on the gentamicin selection plate indicating successful integration of the plasmid into the chromosome (single cross-over event). These gentamicin-resistant colonies were transferred onto a new antibiotic plate and three single colonies were grown overnight (O/N) separately in 5 mL low salt (5% NaCl) liquid Luria Broth (LB), and 50 μL of this O/N culture was diluted 10⁻³ times and was spread on 6% sucrose selection plate for 24-48 hours at 30° C. (double-cross over event). A few colonies found growing on sucrose plates were replica plated on both gentamicin and plain LB plates. Colonies found growing only on plain LB plates were tested for deletion of the desired gene by PCR. Both internal and external sets of primers were used to screen out false positive mutants (due to possible mutations on SacB and GenR cassettes), and colonies showing successful deletion of the gene (only shortened PCR amplicons compared to control) were tested for desired phenotypes (such as defect for growth on glucose in minimal media).

For the integration of furan-metabolizing genes (hmf operon) at glk and gcd/oprB-II sites, the triple gene knockout mutant (oprB-II-gcd⁻/glk⁻) of Pseudomonas putida DSM 6125 (a parent strain of KT2440) defective in utilizing glucose as a carbon source was selected as a host for chromosomal integration of furfural and HMF metabolizing genes (see Table 3) from other bacteria. The furfural metabolizing cluster containing the hmfABCDE genes and the cluster involved in the metabolism of HMF to 2-furoic acid containing hmfFGH genes were sequentially PCR amplified using the genomic DNA of Cupriavidus basilensis using the Q5 DNA polymerase. Other genes such as major facilitator superfamily transporter (MFS; hmfT1, hmfT2) present in these two hmf operons were also PCR amplified as these genes could be potentially involved in the transport of furans. The complete operons were amplified in multiple fragments of ˜3-3.5 kb size (hmfA, hmfBCD, hmfET1, hmfFGH′, hmfHS, and hmfT2) using sets of primers with overlapping sequences at their 5′ and 3′ ends for ease of cloning with repair arms (0.5 kb DNA amplified from up- and down-stream of deleted genes of P. putida).

Gibson assembly method was used to ligate the PCR products (hmf fragments and corresponding repair arms) into a plasmid (pACRISPR) linearized by restriction enzymes (XbaI+HindII) as per the instruction manual for NEBuilder HiFi DNA assembly cloning kit (NEB, USA). The ligated product was transformed into E. coli DH5α and transformants were selected on gentamycin (50 μg/ml). The single colonies obtained from overnight growth were streaked on new antibiotic plates. Clones were confirmed by colony PCR, plasmids were isolated from three positive colonies, and sent for sequencing (Eurofins genomic, USA). Plasmids harboring desired genes with their repair arms were electroporated for the first round of integration of hmfFGT at the glk⁻ deletion site (in-frame to the translation start codon of glk) in P. putida using the homologous recombination process used previously for the deletion of genes. After confirmation of the successful integration of hmfFGH′ genes, a second plasmid construct with hmfHS was being integrated next to hmfFGH′, as well as an additional construct with hmfT2. Similarly, genes for furfural metabolism were integrated at the gcd/oprB-II site in P. putida.

The triple deletion mutant (oprB-II⁻/gcd⁻/glk⁻) of P. putida DSM 6125, hereafter referred to as P. putida_control, and the triple deletion mutant with the integrated furfural and 5-hydroxymethylfurfural (HMF) catabolic operons identified in Table 3 above, hereafter referred to as P. putida_AT-FT, were assessed for furfural and HMF catabolism in medium containing 10-40 mM furfural or HMF and 20 mM glucose. In all experiments, furfural/HMF and glucose concentrations were measured during and after cultivation of both strains of P. putida.

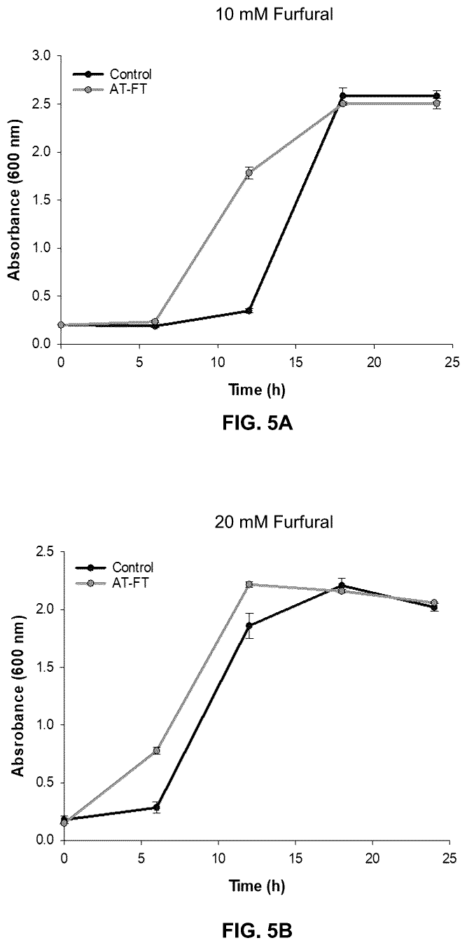

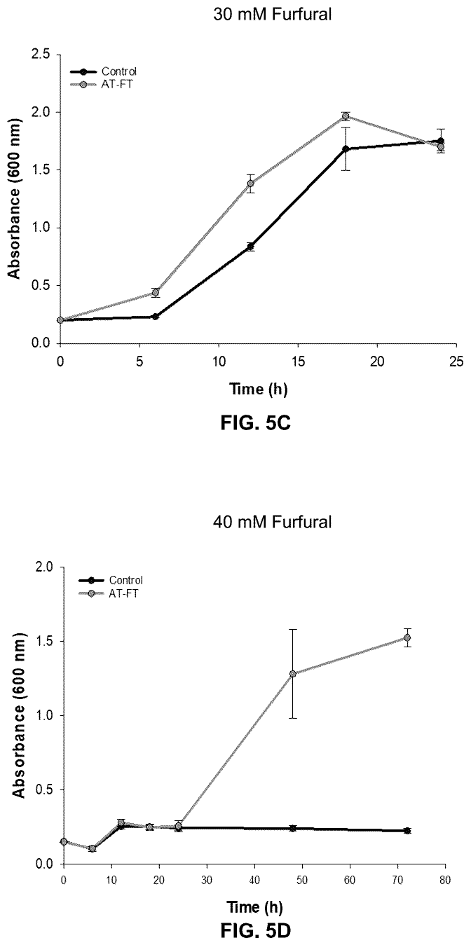

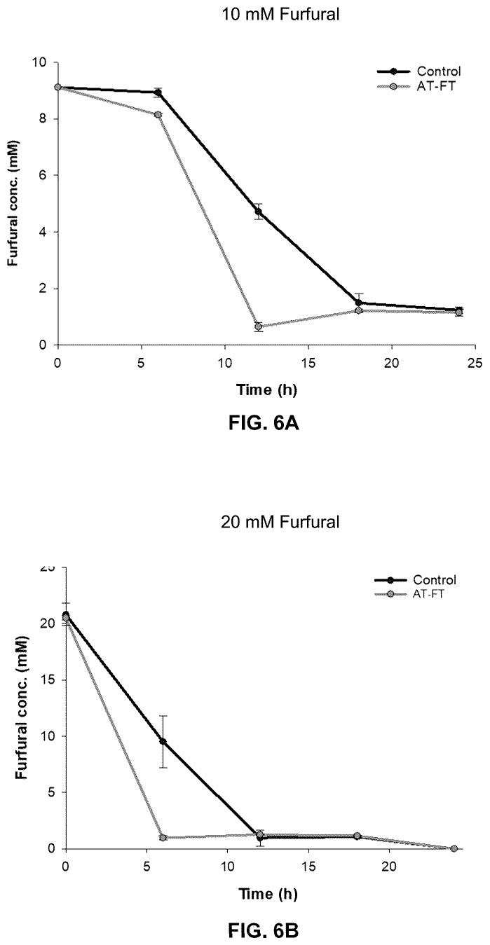

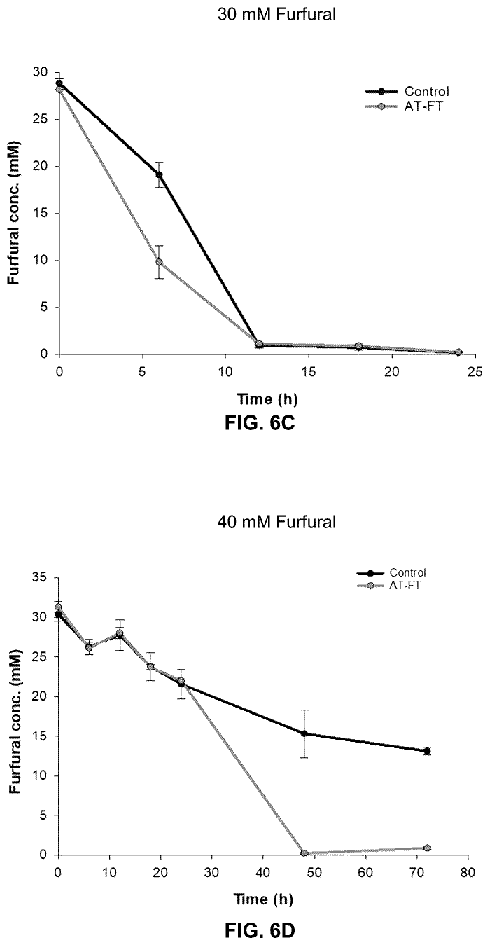

The results show that P. putida_control, in which the glucose import and catabolic machinery was disrupted, is capable of furfural and HMF catabolism. Notably, P. putida_AT-FT, in which the glucose import and catabolic machinery was disrupted in addition to incorporation of the furfural and HMF catabolic operons into the chromosome, exhibited a significantly higher rate of growth and inhibitor utilization at all the inhibitor concentrations tested. With P. putida_AT-FT, the growth rate was 5.5-, 2.6-, 1.8-, and 6.7-fold greater than the rates observed with P. putida_control in cultures supplemented with 10, 20, 30, and 40 mM furfural, respectively (FIGS. 5A-5D). Concomitantly, the rates of furfural reduction by P. putida_AT-FT were 46-, 20-, 2.5-, and 330-fold faster than those observed for P. putida_control (FIGS. 6A-6D).

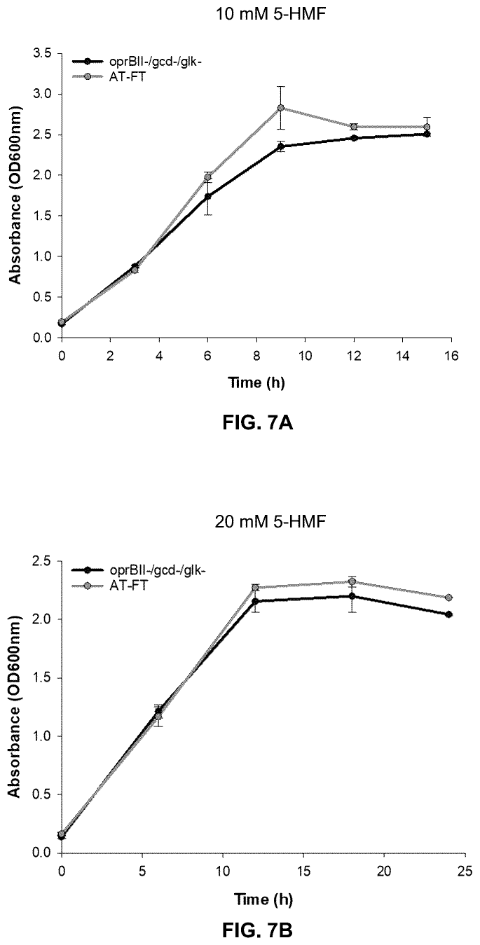

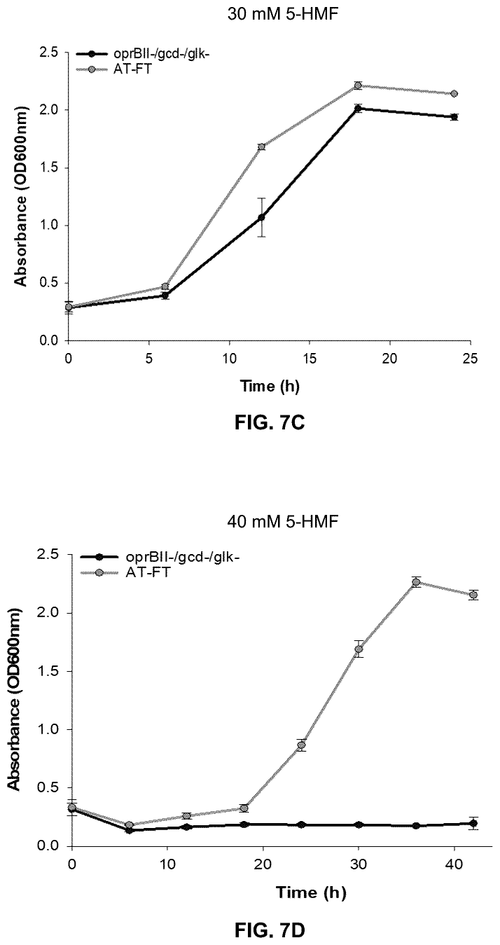

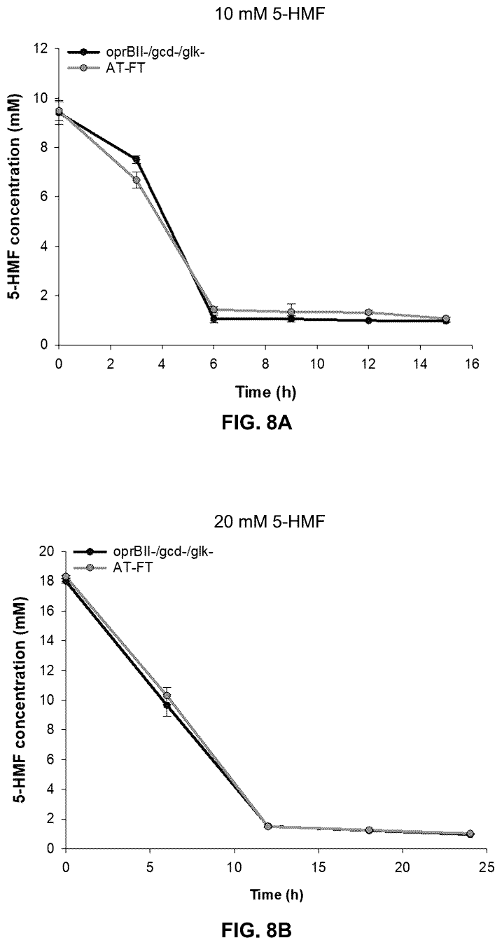

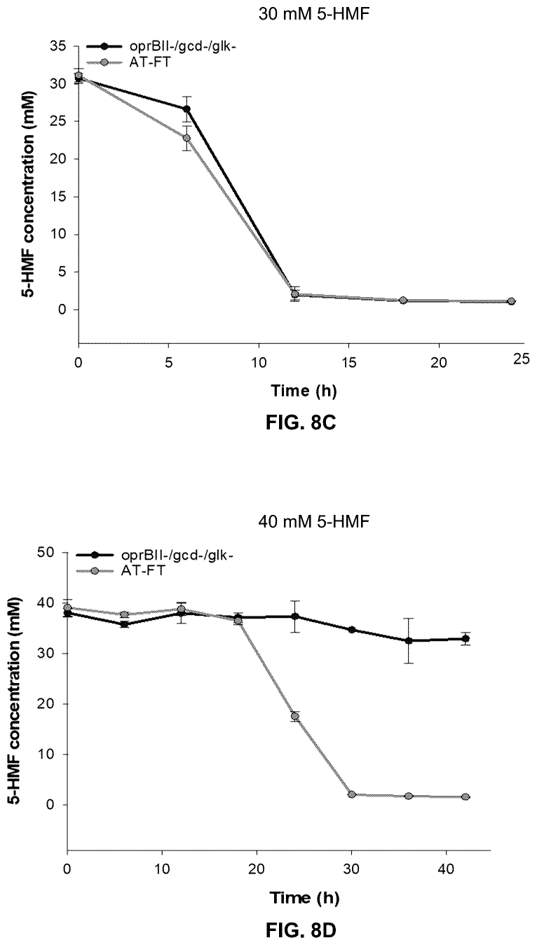

When HMF (10, 20, 30, and 40 mM) was supplemented to cultures of both strains of P. putida, both strains exhibited the capacity to reduce this inhibitor. However, with increasing HMF concentration, the ability of P. putida_AT-FT to better catabolize HMF became apparent (FIGS. 7A-7D and 8A-8D). With 10 and 20 mM HMF, the growth rates of P. putida_AT-FT were marginally greater than those of P. putida_control (FIGS. 7A and 7B). When HMF concentrations were increased to 30 and 40 mM, the growth rates of P. putida_AT-FT were 1.8- and 8.0-fold greater than those of P. putida_control (FIGS. 7C and 7D). In fact, at 40 mM HMF, the growth of P. putida_control was inhibited (FIG. 7D). Similar to the growth profiles observed with HMF, at 10, 20, and 30 mM HMF, both strains exhibited comparable rates of HMF utilization (FIGS. 8A-8C). However, with 40 mM HMF, HMF concentration reduced 1,915-fold faster in cultures of P. putida_AT-FT relative to those of P. putida_control (FIG. 8D). In both cases (with furfural or HMF), the glucose concentration remained intact (i.e., unused).

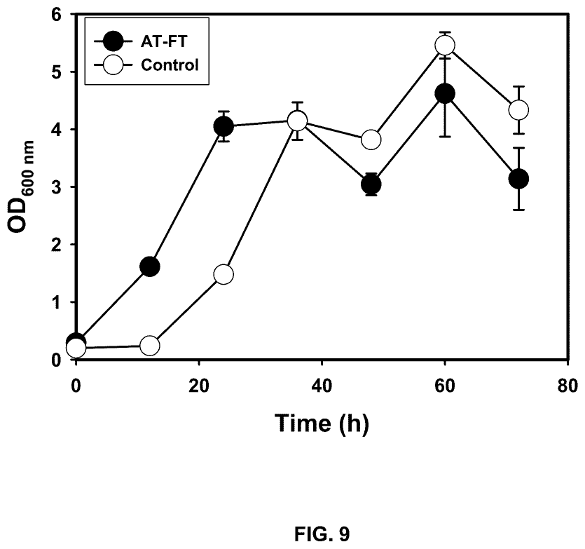

To ascertain the potential utility of using P. putida_AT-FT for selective removal of inhibitors in lignocellulosic hydrolysate (LBH), to facilitate subsequent fermentation of LBH-borne sugars to target products/chemicals, P. putida_AT-FT and P. putida_control were grown in a medium containing 40 mM furfural and 60 g/L glucose for 12 hours. Because furfural is the most abundant inhibitor in LBH, it was selected for this preliminary assessment. Afterwards, the resulting medium was sterilized by standard procedures and then fermented with the butanol-producing Clostridium beijerinckii. The growth profile of C. beijerinckii in media in which P. putida_AT-FT and P. putida_control were pre-grown underscore more enhanced furfural utilization, hence, removal from the P. putida_AT-FT-pre-grown medium relative to the P. putida_control-pre-grown medium. Specifically, C. beijerinckii exhibited a 2-fold faster growth rate in the medium in which P. putida_AT-FT was pre-grown when compared to that in which P. putida_control was pre-grown (FIG. 9). However, considerable sugar losses were observed for both sets of cultures. To ensure absolute selective inhibitor removal without sugar utilization at high glucose concentration-such as 60 g/L used in this study, which mimics LBH-we have identified three additional genes that we will knock out individually and in combination in P. putida_AT-FT to ensure complete shutdown of glucose import and utilization. These are oprB-1 of Pseudomonas putida (SEQ ID NO:45 (coding sequence), SEQ ID NO:46 (protein sequence)), gtsB of Pseudomonas putida (SEQ ID NO:47 (coding sequence), SEQ ID NO:48 (protein sequence)), and KBDANE_14125 or PP_RS13865 of Pseudomonas putida (SEQ ID NO:49 (coding sequence), SEQ ID NO:50 (protein sequence)).

Primers used for deletion of Glk and OprB-II/
GCD genes:
Glk AF′-XhoI:
(SEQ ID NO: 37)
5′-CCGCTCGAGGTGTTCCAGGACCAGCAGTC-3′
Glk AR′:
(SEQ ID NO: 38)
5′-ACGCCTGCTGCCAACCAGCAGGTGCTTCAT-3′
Glk BF′:
(SEQ ID NO: 39)
5′-TGCTGGTTGGCAGCAGGCGTTGGATCACTGA-3′
Glk BR′-HindIII:
(SEQ ID NO: 40)
5′-CCCAAGCTTCAGTCGTCGAAGGCCAGCA-3′
OprB-II-AF-XhoI:
(SEQ ID NO: 41)
5′-CCCAAGCTTGTAGACGTGCAGCACGCTG-3′
OprB-II-AR:
(SEQ ID NO: 42)
5′-CTCGGCTAAAGGCAGTTGGAACATGAGATAGC-3′
GCD-BF:
(SEQ ID NO: 43)
5′-CAACTGCCTTTAGCCGAGTAAGCGACACC-3′
GCD-BR-HindIII:
(SEQ ID NO: 44)
5′-CCGCTCGAGGCAGTGCCGAGGTGTCGAAG-3′

### EXEMPLARY EMBODIMENTS

1. A recombinant microorganism comprising one or more modifications with respect to a corresponding microorganism not comprising the one or more modifications, wherein the one or more modifications comprise 1 or more, 2 or more, 3 or more, 4 or more, 5 or more, 6 or more, 7 or more, 8 or more, 9 or more, 10 or more, 11 or more, 12 or more, 13 or more, 14 or more, or each of:
   a functional deletion of a glucokinase gene present in the corresponding microorganism;
   a functional deletion of a quinoprotein glucose dehydrogenase gene present in the corresponding microorganism;
   a functional deletion of a carbohydrate transporter gene present in the corresponding microorganism;
   a recombinant gene encoding HmfA of Cupriavidus basilensis or a homolog thereof;
   a recombinant gene encoding HmfB of Cupriavidus basilensis or a homolog thereof;
   a recombinant gene encoding HmfC of Cupriavidus basilensis or a homolog thereof;
   a recombinant gene encoding HmfD of Cupriavidus basilensis or a homolog thereof;
   a recombinant gene encoding HmfE of Cupriavidus basilensis or a homolog thereof;
   a recombinant gene encoding HmfT1 of Cupriavidus basilensis or a homolog thereof;
   a recombinant gene encoding HmfF of Cupriavidus basilensis or a homolog thereof;
   a recombinant gene encoding HmfG of Cupriavidus basilensis or a homolog thereof;
   a recombinant gene encoding HmfH′ of Cupriavidus basilensis or a homolog thereof;
   a recombinant gene encoding HmfH of Cupriavidus basilensis or a homolog thereof;
   a recombinant gene encoding HmfS of Cupriavidus basilensis or a homolog thereof; and
   a recombinant gene encoding HmfT2 of Cupriavidus basilensis or a homolog thereof.
2. The recombinant microorganism of exemplary embodiment 1, wherein the one or more modifications comprise one or more, two or more, or each of:
   a functional deletion of a glucokinase gene present in the corresponding microorganism;
   a functional deletion of a quinoprotein glucose dehydrogenase gene present in the corresponding microorganism; and
   a functional deletion of a carbohydrate transporter gene present in the corresponding microorganism.
3. The recombinant microorganism of any prior exemplary embodiment, wherein the one or more modifications comprise one or both of:
   a functional deletion of a glucokinase gene present in the corresponding microorganism; and
   a functional deletion of a quinoprotein glucose dehydrogenase gene present in the corresponding microorganism.
4. The recombinant microorganism of any prior exemplary embodiment, wherein:
   the glucokinase gene is glk of Pseudomonas putida, glk of Escherichia coli, glk of Enterobacter hormaechei, or a homolog of any of the foregoing;
   the quinoprotein glucose dehydrogenase gene is gcd of Pseudomonas putida, gcd of Escherichia coli, gcd of Enterobacter hormaechei, or a homolog of any of the foregoing; and/or
   the carbohydrate transporter gene is oprB-II of Pseudomonas putida or a homolog thereof.
5. The recombinant microorganism of any prior exemplary embodiment, wherein:
   the glucokinase gene encodes a protein comprising an amino acid sequence with at least 80% sequence identity to a sequence selected from the group consisting of SEQ ID NOS:2 and 8;
   the quinoprotein glucose dehydrogenase gene encodes a protein comprising an amino acid sequence with at least 80% sequence identity to a sequence selected from the group consisting of SEQ ID NOS:4 and 10; and
   the carbohydrate transporter gene encodes a protein comprising an amino acid sequence with at least 80% sequence identity to a sequence selected from the group consisting of SEQ ID NOS:6, 46, 48, and 50.
6. The recombinant microorganism of any prior exemplary embodiment, wherein:
   the glucokinase gene encodes a protein comprising an amino acid sequence with at least 95% sequence identity to a sequence selected from the group consisting of SEQ ID NOS:2 and 8;
   the quinoprotein glucose dehydrogenase gene encodes a protein comprising an amino acid sequence with at least 95% sequence identity to a sequence selected from the group consisting of SEQ ID NOS:4 and 10; and
   the carbohydrate transporter gene encodes a protein comprising an amino acid sequence with at least 95% sequence identity to a sequence selected from the group consisting of SEQ ID NOS:6, 46, 48, and 50.
7. The recombinant microorganism of any prior exemplary embodiment, wherein the wherein the one or more modifications comprise a functional deletion of a carbohydrate transporter gene present in the corresponding microorganism, wherein the carbohydrate transporter gene encodes a protein comprising an amino acid sequence with at least 80% or at least 95% sequence identity to SEQ ID NO:6.
8. The recombinant microorganism of exemplary embodiment 7, further comprising a functional deletion of one, two or three additional carbohydrate transporter genes present in the corresponding microorganism, wherein the additional carbohydrate transporter genes each encode a protein comprising an amino acid sequence with at least 80% or at least 95% sequence identity to a sequence selected from the group consisting of SEQ ID NOS:46, 48, and 50.
9. The recombinant microorganism of exemplary embodiment 7, further comprising a functional deletion of three additional carbohydrate transporter genes present in the corresponding microorganism, wherein the additional carbohydrate transporter genes encode:
   a protein comprising an amino acid sequence with at least 80% or at least 95% sequence identity to SEQ ID NO:46;
   a protein comprising an amino acid sequence with at least 80% or at least 95% sequence identity to SEQ ID NO: 48;
a protein comprising an amino acid sequence with at least 80% or at least 95% sequence identity to SEQ ID NO:50.
10. The recombinant microorganism of any prior exemplary embodiment, wherein the one or more modifications comprise one or more, two or more three or more, four or more, five or more, or each of:
   a recombinant gene encoding HmfA of Cupriavidus basilensis or a homolog thereof;
   a recombinant gene encoding HmfB of Cupriavidus basilensis or a homolog thereof;
   a recombinant gene encoding HmfC of Cupriavidus basilensis or a homolog thereof;
   a recombinant gene encoding HmfD of Cupriavidus basilensis or a homolog thereof;
   a recombinant gene encoding HmfE of Cupriavidus basilensis or a homolog thereof; and
   a recombinant gene encoding HmfT1 of Cupriavidus basilensis or a homolog thereof.
11. The recombinant microorganism of any prior exemplary embodiment, wherein the one or more modifications comprise one or more, two or more three or more, four or more, five or more, or each of:
   a recombinant gene encoding HmfF of Cupriavidus basilensis or a homolog thereof;
   a recombinant gene encoding HmfG of Cupriavidus basilensis or a homolog thereof;
   a recombinant gene encoding HmfH′ of Cupriavidus basilensis or a homolog thereof;
   a recombinant gene encoding HmfH of Cupriavidus basilensis or a homolog thereof;
   a recombinant gene encoding HmfS of Cupriavidus basilensis or a homolog thereof; and
   a recombinant gene encoding HmfT2 of Cupriavidus basilensis or a homolog thereof.
12. The recombinant microorganism of any prior exemplary embodiment, wherein:
   the HmfA of Cupriavidus basilensis or the homolog thereof comprises an amino acid sequence with at least 80% sequence identity to SEQ ID NO:13;
   the HmfB of Cupriavidus basilensis or the homolog thereof comprises an amino acid sequence with at least 80% sequence identity to SEQ ID NO:15;
   the HmfC of Cupriavidus basilensis or the homolog thereof comprises an amino acid sequence with at least 80% sequence identity to SEQ ID NO:17;
   the HmfD of Cupriavidus basilensis or the homolog thereof comprises an amino acid sequence with at least 80% sequence identity to SEQ ID NO:19;
   the HmfE of Cupriavidus basilensis or the homolog thereof comprises an amino acid sequence with at least 80% sequence identity to SEQ ID NO:21;
   the HmfT1 of Cupriavidus basilensis or the homolog thereof comprises an amino acid sequence with at least 80% sequence identity to SEQ ID NO:23;
   the HmfF of Cupriavidus basilensis or the homolog thereof comprises an amino acid sequence with at least 80% sequence identity to SEQ ID NO:26;
   the HmfG of Cupriavidus basilensis or the homolog thereof comprises an amino acid sequence with at least 80% sequence identity to SEQ ID NO:28;
   the HmfH′ of Cupriavidus basilensis or the homolog thereof comprises an amino acid sequence with at least 80% sequence identity to SEQ ID NO:30;
   the HmfH of Cupriavidus basilensis or the homolog thereof comprises an amino acid sequence with at least 80% sequence identity to SEQ ID NO:32;
   the HmfS of Cupriavidus basilensis or the homolog thereof comprises an amino acid sequence with at least 80% sequence identity to SEQ ID NO:34; and
   the HmfT2 of Cupriavidus basilensis or the homolog thereof comprises an amino acid sequence with at least 80% sequence identity to SEQ ID NO:36.
13. The recombinant microorganism of any prior exemplary embodiment, wherein:
   the HmfA of Cupriavidus basilensis or the homolog thereof comprises an amino acid sequence with at least 95% sequence identity to SEQ ID NO:13;
   the HmfB of Cupriavidus basilensis or the homolog thereof comprises an amino acid sequence with at least 95% sequence identity to SEQ ID NO:15;
   the HmfC of Cupriavidus basilensis or the homolog thereof comprises an amino acid sequence with at least 95% sequence identity to SEQ ID NO:17;
   the HmfD of Cupriavidus basilensis or the homolog thereof comprises an amino acid sequence with at least 95% sequence identity to SEQ ID NO:19;
   the HmfE of Cupriavidus basilensis or the homolog thereof comprises an amino acid sequence with at least 95% sequence identity to SEQ ID NO:21;
   the HmfT1 of Cupriavidus basilensis or the homolog thereof comprises an amino acid sequence with at least 95% sequence identity to SEQ ID NO:23;
   the HmfF of Cupriavidus basilensis or the homolog thereof comprises an amino acid sequence with at least 95% sequence identity to SEQ ID NO:26;
   the HmfG of Cupriavidus basilensis or the homolog thereof comprises an amino acid sequence with at least 95% sequence identity to SEQ ID NO:28;
   the HmfH′ of Cupriavidus basilensis or the homolog thereof comprises an amino acid sequence with at least 95% sequence identity to SEQ ID NO:30;
   the HmfH of Cupriavidus basilensis or the homolog thereof comprises an amino acid sequence with at least 95% sequence identity to SEQ ID NO:32;
   the HmfS of Cupriavidus basilensis or the homolog thereof comprises an amino acid sequence with at least 95% sequence identity to SEQ ID NO:34; and
   the HmfT2 of Cupriavidus basilensis or the homolog thereof comprises an amino acid sequence with at least 95% sequence identity to SEQ ID NO:36.
14. The recombinant microorganism of any prior exemplary embodiment, wherein the recombinant microorganism is an aerobic microorganism.
15. The recombinant microorganism of any prior exemplary embodiment, wherein the recombinant microorganism is a bacterium.
16. The recombinant microorganism of any prior exemplary embodiment, wherein the recombinant microorganism is an aerobic bacterium.
17. The recombinant microorganism of any prior exemplary embodiment, wherein the recombinant microorganism is from a genus selected from the group consisting of Pseudomonas, Escherichia, and Enterobacter.
18. The recombinant microorganism of any prior exemplary embodiment, wherein the recombinant microorganism is selected from the group consisting of Pseudomonas putida, Escherichia coli, and Enterobacter hormaechei.
19. The recombinant microorganism of any prior exemplary embodiment, wherein the recombinant microorganism is from a genus of Pseudomonas.
20. The recombinant microorganism of any prior exemplary embodiment, wherein the recombinant microorganism is Pseudomonas putida.
21. The recombinant microorganism of any prior exemplary embodiment, wherein the recombinant microorganism exhibits reduced consumption of a carbohydrate with respect to the corresponding microorganism.
22. The recombinant microorganism of any prior exemplary embodiment, wherein the recombinant microorganism exhibits increased consumption of a substituted furan with respect to the corresponding microorganism.
23. The recombinant microorganism of any prior exemplary embodiment, wherein the recombinant microorganism exhibits increased consumption of a substituted furan selected from the group consisting of furfural and hydroxymethylfurfural with respect to the corresponding microorganism.
24. A method of decreasing an amount of a substituted furan in a medium, the method comprising contacting the medium with the recombinant microorganism of any one of exemplary embodiments 1-23 for a time sufficient to decrease the substituted furan in the medium.
25. The method of exemplary embodiment 24, wherein the substituted furan is selected from the group consisting of furfural and hydroxymethylfurfural.
26. The method of any one of exemplary embodiments 24-25, wherein the medium comprises lignocellulosic biomass.
27. The method of any one of exemplary embodiments 24-26, wherein the medium comprises lignocellulosic biomass hydrolysate.
28. The method of any one of exemplary embodiments 24-27, wherein the contacting is performed under aerobic conditions.

What is claimed is:

1. A recombinant microorganism comprising one or more modifications with respect to a corresponding microorganism not comprising the one or more modifications, wherein the one or more modifications comprise 1 or more, 2 or more, 3 or more, 4 or more, 5 or more, 6 or more, 7 or more, 8 or more, 9 or more, 10 or more, 11 or more, 12 or more, 13 or more, 14 or more, or each of:
a functional deletion of a glucokinase gene present in the corresponding microorganism;
a functional deletion of a quinoprotein glucose dehydrogenase gene present in the corresponding microorganism;
a functional deletion of a carbohydrate transporter gene present in the corresponding microorganism;
a recombinant gene encoding HmfA of Cupriavidus basilensis or a homolog thereof;
a recombinant gene encoding HmfB of Cupriavidus basilensis or a homolog thereof;
a recombinant gene encoding HmfC of Cupriavidus basilensis or a homolog thereof;
a recombinant gene encoding HmfD of Cupriavidus basilensis or a homolog thereof;
a recombinant gene encoding HmfE of Cupriavidus basilensis or a homolog thereof;
a recombinant gene encoding HmfT1 of Cupriavidus basilensis or a homolog thereof;
a recombinant gene encoding HmfF of Cupriavidus basilensis or a homolog thereof;
a recombinant gene encoding HmfG of Cupriavidus basilensis or a homolog thereof;
a recombinant gene encoding HmfH′ of Cupriavidus basilensis or a homolog thereof;
a recombinant gene encoding HmfH of Cupriavidus basilensis or a homolog thereof;
a recombinant gene encoding HmfS of Cupriavidus basilensis or a homolog thereof; and
a recombinant gene encoding HmfT2 of Cupriavidus basilensis or a homolog thereof.

2. The recombinant microorganism of claim 1, wherein the one or more modifications comprise each of:
a functional deletion of a glucokinase gene present in the corresponding microorganism; and
a functional deletion of a quinoprotein glucose dehydrogenase gene present in the corresponding microorganism; and
a functional deletion of a carbohydrate transporter gene present in the corresponding microorganism.

3. The recombinant microorganism of claim 2, wherein:
the glucokinase gene is glk of Pseudomonas putida, glk of Escherichia coli, glk of Enterobacter hormaechei, or a homolog of any of the foregoing;
the quinoprotein glucose dehydrogenase gene is gcd of Pseudomonas putida, gcd of Escherichia coli, gcd of Enterobacter hormaechei, or a homolog of any of the foregoing; and
the carbohydrate transporter gene is oprB-II of Pseudomonas putida or a homolog thereof.

4. The recombinant microorganism of claim 2, wherein:
the glucokinase gene encodes a protein comprising an amino acid sequence with at least 95% sequence identity to a sequence selected from the group consisting of SEQ ID NOS:2 and 8;
the quinoprotein glucose dehydrogenase gene encodes a protein comprising an amino acid sequence with at least 95% sequence identity to a sequence selected from the group consisting of SEQ ID NOS:4 and 10; and
the carbohydrate transporter gene encodes a protein comprising an amino acid sequence with at least 95% sequence identity to a sequence selected from the group consisting of SEQ ID NOS:6, 46, 48, and 50.

5. The recombinant microorganism of claim 4, wherein the carbohydrate transporter gene encodes a protein comprising an amino acid sequence with at least 95% sequence identity to SEQ ID NO:6.

6. The recombinant microorganism of claim 5, further comprising a functional deletion of one, two or three additional carbohydrate transporter genes present in the corresponding microorganism, wherein the additional carbohydrate transporter genes each encode a protein comprising an amino acid sequence with at least 95% sequence identity to a sequence selected from the group consisting of SEQ ID NOS:46, 48, and 50.

7. The recombinant microorganism of claim 5, further comprising a functional deletion of three additional carbohydrate transporter genes present in the corresponding microorganism, wherein the additional carbohydrate transporter genes encode:
a protein comprising an amino acid sequence with at least 95% sequence identity to SEQ ID NO:46;
a protein comprising an amino acid sequence with at least 95% sequence identity to SEQ ID NO: 48; and
a protein comprising an amino acid sequence with at least 95% sequence identity to SEQ ID NO:50.

8. The recombinant microorganism of claim 1, wherein the one or more modifications comprise one or more of:
a recombinant gene encoding HmfA of Cupriavidus basilensis or a homolog thereof;
a recombinant gene encoding HmfB of Cupriavidus basilensis or a homolog thereof;
a recombinant gene encoding HmfC of Cupriavidus basilensis or a homolog thereof;
a recombinant gene encoding HmfD of Cupriavidus basilensis or a homolog thereof;
a recombinant gene encoding HmfE of Cupriavidus basilensis or a homolog thereof;
a recombinant gene encoding HmfT1 of Cupriavidus basilensis or a homolog thereof;
a recombinant gene encoding HmfF of Cupriavidus basilensis or a homolog thereof;
a recombinant gene encoding HmfG of Cupriavidus basilensis or a homolog thereof;
a recombinant gene encoding HmfH′ of Cupriavidus basilensis or a homolog thereof;
a recombinant gene encoding HmfH of Cupriavidus basilensis or a homolog thereof;
a recombinant gene encoding HmfS of Cupriavidus basilensis or a homolog thereof; and
a recombinant gene encoding HmfT2 of Cupriavidus basilensis or a homolog thereof.

9. The recombinant microorganism of claim 1, wherein the one or more modifications comprise each of:
a recombinant gene encoding HmfA of Cupriavidus basilensis or a homolog thereof;
a recombinant gene encoding HmfB of Cupriavidus basilensis or a homolog thereof;
a recombinant gene encoding HmfC of Cupriavidus basilensis or a homolog thereof;
a recombinant gene encoding HmfD of Cupriavidus basilensis or a homolog thereof;
a recombinant gene encoding HmfE of Cupriavidus basilensis or a homolog thereof;
a recombinant gene encoding HmfT1 of Cupriavidus basilensis or a homolog thereof;
a recombinant gene encoding HmfF of Cupriavidus basilensis or a homolog thereof;
a recombinant gene encoding HmfG of Cupriavidus basilensis or a homolog thereof;
a recombinant gene encoding HmfH′ of Cupriavidus basilensis or a homolog thereof;
a recombinant gene encoding HmfH of Cupriavidus basilensis or a homolog thereof;
a recombinant gene encoding HmfS of Cupriavidus basilensis or a homolog thereof; and
a recombinant gene encoding HmfT2 of Cupriavidus basilensis or a homolog thereof.

10. The recombinant microorganism of claim 9, wherein:
the HmfA of Cupriavidus basilensis or the homolog thereof comprises an amino acid sequence with at least 95% sequence identity to SEQ ID NO:13;
the HmfB of Cupriavidus basilensis or the homolog thereof comprises an amino acid sequence with at least 95% sequence identity to SEQ ID NO:15;
the HmfC of Cupriavidus basilensis or the homolog thereof comprises an amino acid sequence with at least 95% sequence identity to SEQ ID NO:17;
the HmfD of Cupriavidus basilensis or the homolog thereof comprises an amino acid sequence with at least 95% sequence identity to SEQ ID NO:19;
the HmfE of Cupriavidus basilensis or the homolog thereof comprises an amino acid sequence with at least 95% sequence identity to SEQ ID NO:21;
the HmfT1 of Cupriavidus basilensis or the homolog thereof comprises an amino acid sequence with at least 95% sequence identity to SEQ ID NO:23;
the HmfF of Cupriavidus basilensis or the homolog thereof comprises an amino acid sequence with at least 95% sequence identity to SEQ ID NO:26;
the HmfG of Cupriavidus basilensis or the homolog thereof comprises an amino acid sequence with at least 95% sequence identity to SEQ ID NO:28;
the HmfH′ of Cupriavidus basilensis or the homolog thereof comprises an amino acid sequence with at least 95% sequence identity to SEQ ID NO:30;
the HmfH of Cupriavidus basilensis or the homolog thereof comprises an amino acid sequence with at least 95% sequence identity to SEQ ID NO:32;
the HmfS of Cupriavidus basilensis or the homolog thereof comprises an amino acid sequence with at least 95% sequence identity to SEQ ID NO:34; and
the HmfT2 of Cupriavidus basilensis or the homolog thereof comprises an amino acid sequence with at least 95% sequence identity to SEQ ID NO:36.

11. The recombinant microorganism of claim 1, wherein the recombinant microorganism is an aerobic bacterium.

12. The recombinant microorganism of claim 1, wherein the recombinant microorganism is from a genus selected from the group consisting of Pseudomonas, Escherichia, and Enterobacter.

13. The recombinant microorganism of claim 1, wherein:
the one or more modifications comprise:
a functional deletion of a glucokinase gene present in the corresponding microorganism, wherein the glucokinase gene encodes a protein comprising an amino acid sequence with at least 95% sequence identity to SEQ ID NO:2;
a functional deletion of a quinoprotein glucose dehydrogenase gene present in the corresponding microorganism, wherein the quinoprotein glucose dehydrogenase gene encodes a protein comprising an amino acid sequence with at least 95% sequence identity to SEQ ID NO:4;
a functional deletion of a carbohydrate transporter gene present in the corresponding microorganism, wherein the carbohydrate transporter gene encodes a protein comprising an amino acid sequence with at least 95% sequence identity to SEQ ID NO:6;
a recombinant gene encoding HmfA of Cupriavidus basilensis or a homolog thereof, wherein the HmfA of Cupriavidus basilensis or the homolog thereof comprises an amino acid sequence with at least 95% sequence identity to SEQ ID NO:13;
a recombinant gene encoding HmfB of Cupriavidus basilensis or a homolog thereof, wherein the HmfB of Cupriavidus basilensis or the homolog thereof comprises an amino acid sequence with at least 95% sequence identity to SEQ ID NO:15;
a recombinant gene encoding HmfC of Cupriavidus basilensis or a homolog thereof, wherein the HmfC of Cupriavidus basilensis or the homolog thereof comprises an amino acid sequence with at least 95% sequence identity to SEQ ID NO:17;
a recombinant gene encoding HmfD of Cupriavidus basilensis or a homolog thereof, wherein the HmfD of Cupriavidus basilensis or the homolog thereof comprises an amino acid sequence with at least 95% sequence identity to SEQ ID NO:19;
a recombinant gene encoding HmfE of Cupriavidus basilensis or a homolog thereof, wherein the HmfE of Cupriavidus basilensis or the homolog thereof comprises an amino acid sequence with at least 95% sequence identity to SEQ ID NO:21;
a recombinant gene encoding HmfT1 of Cupriavidus basilensis or a homolog thereof, wherein the HmfT1 of Cupriavidus basilensis or the homolog thereof comprises an amino acid sequence with at least 95% sequence identity to SEQ ID NO:23;
a recombinant gene encoding HmfF of Cupriavidus basilensis or a homolog thereof, wherein the HmfF of Cupriavidus basilensis or the homolog thereof comprises an amino acid sequence with at least 95% sequence identity to SEQ ID NO:26;
a recombinant gene encoding HmfG of Cupriavidus basilensis or a homolog thereof, wherein the HmfG of Cupriavidus basilensis or the homolog thereof comprises an amino acid sequence with at least 95% sequence identity to SEQ ID NO:28;
a recombinant gene encoding HmfH′ of Cupriavidus basilensis or a homolog thereof, wherein the HmfH′ of Cupriavidus basilensis or the homolog thereof comprises an amino acid sequence with at least 95% sequence identity to SEQ ID NO:30;
a recombinant gene encoding HmfH of Cupriavidus basilensis or a homolog thereof, wherein the HmfH of Cupriavidus basilensis or the homolog thereof comprises an amino acid sequence with at least 95% sequence identity to SEQ ID NO:32;
a recombinant gene encoding HmfS of Cupriavidus basilensis or a homolog thereof, wherein the HmfS of Cupriavidus basilensis or the homolog thereof comprises an amino acid sequence with at least 95% sequence identity to SEQ ID NO:34; and
a recombinant gene encoding HmfT2 of Cupriavidus basilensis or a homolog thereof, wherein the HmfT2 of Cupriavidus basilensis or the homolog thereof comprises an amino acid sequence with at least 95% sequence identity to SEQ ID NO:36;
the recombinant microorganism is recombinant Pseudomonas putida;
the corresponding microorganism is native Pseudomonas putida;
the recombinant microorganism exhibits reduced consumption of a carbohydrate with respect to the corresponding microorganism; and
the recombinant microorganism exhibits increased consumption of a substituted furan with respect to the corresponding microorganism.

14. The recombinant microorganism of claim 13, further comprising a functional deletion of three additional carbohydrate transporter genes present in the corresponding microorganism, wherein the additional carbohydrate transporter genes encode:
a protein comprising an amino acid sequence with at least 95% sequence identity to SEQ ID NO:46;
a protein comprising an amino acid sequence with at least 95% sequence identity to SEQ ID NO: 48;
a protein comprising an amino acid sequence with at least 95% sequence identity to SEQ ID NO:50.

15. A method of decreasing an amount of a substituted furan in a medium, the method comprising contacting the medium with the recombinant microorganism of claim 1 for a time sufficient to decrease the substituted furan in the medium.

16. The method of claim 15, wherein the substituted furan is selected from the group consisting of furfural and hydroxymethylfurfural.

17. The method of claim 15, wherein the medium comprises lignocellulosic biomass hydrolysate.

18. The method of claim 15, wherein the contacting is performed under aerobic conditions.
